(19) United States
(12) Patent Application Publication
(10) Pub. No.: US 2015/0337279 A1
(43) Pub. Date: Nov. 26, 2015

(54) RECOMBINANT FUNGAL POLYPEPTIDES

(71) Applicant: CODEXIS, INC., Redwood City, CA (US)

(72) Inventors: Xiyun Zhang, Fremont, CA (US); Dipnath Baidyaroy, Fremont, CA (US)

(21) Appl. No.: 14/443,464

(22) PCT Filed: Nov. 19, 2013
(86) PCT No.: PCT/US2013/070736

§ 371 Date: May 18, 2015

Related U.S. Application Data
(60) Provisional application No. 61/728,680, filed on Nov. 20, 2012.

(51) Int. Cl. C12N 9/42; C12P 19/02; C12P 19/14

(52) U.S. Cl. CPC C12N 9/2437; C12P 19/14; C12P 19/02; C12Y 302/01004

(57) ABSTRACT

The invention relates to Myceliophthora thermophila biomass degradation polypeptides and Myceliophthora thermophila polypeptides that increase protein productivity, nucleic acids encoding such polypeptides, and methods of producing the polypeptides. The invention further relates to methods for degrading a cellulosic biomass using a biomass degradation polypeptide and methods of engineering a cell or methods of increasing protein production using a polypeptide of the invention.

### CROSS-REFERENCES TO RELATED APPLICATIONS

This application claims the benefit of U.S. Provisional Application No. 61/728,680, filed Nov. 20, 2012, the content of which is incorporated herein by reference in its entirety and for all purposes.

### REFERENCE TO A “SEQUENCE LISTING,” A TABLE, OR A COMPUTER PROGRAM LISTING APPENDIX SUBMITTED AS A TEXT FILE

The Sequence Listing written in file CX35-124WO1_ST25.TXT, created on Nov. 18, 2013, 16,549,083 bytes, machine format IBM-PC, MS Windows operating system, is hereby incorporated by reference.

### FIELD OF THE INVENTION

The invention relates to expression of recombinant Myceliophthora thermophila enzymes involved in biomass degradation and/or enhancing hydrolysis and protein production from cells.

### BACKGROUND OF THE INVENTION

Cellulosic biomass is a significant renewable resource for the generation of sugars. Fermentation of these sugars can yield commercially valuable end-products, including biofuels and chemicals that are currently derived from petroleum. While the fermentation of simple sugars to ethanol is relatively straightforward, the efficient conversion of cellulosic biomass to fermentable sugars such as glucose is challenging. See, e.g., Ladisch et al., 1983, Enzyme Microb. Technol. 5:82. Cellulose may be pretreated chemically, mechanically or in other ways to increase the susceptibility of cellulose to hydrolysis. Such pretreatment may be followed by the enzymatic conversion of cellulose to glucose, cellobiose, cello-oligosaccharides and the like, using enzymes that specialize in breaking down the β-1-4 glycosidic bonds of cellulose. These enzymes are collectively referred to as “cellulases”.

Cellulases are divided into three sub-categories of enzymes: 1,4-β-D-glucan glucanohydrolase (“endoglucanase” or “EG”); 1,4-β-D-glucan cellobiohydrolase (“exoglucanase”, “cellobiohydrolase”, or “CBH”); and (β-D-glucoside-glucohydrolase (“β-glucosidase”, “cellobiase” or “BG”). Endoglucanases randomly attack the interior parts and mainly the amorphous regions of cellulose. Exoglucanases incrementally shorten the glucan molecules by binding to the glucan ends and releasing mainly cellobiose units from the ends of the cellulose polymer. β-glucosidases split the cellobiose, a water-soluble β-1,4-linked dimer of glucose, into two units of glucose. Efficient production of cellulases for use in processing cellulosic biomass would reduce costs and increase the efficiency of production of biofuels and other commercially valuable compounds.

Other enzymes (“accessory enzymes” or “accessory proteins”) also participate in degradation of cellulosic biomass to obtain sugars. These enzymes include esterases, lipases, laccases, and other oxidative enzymes such as oxidoreductases, and the like.

Additional proteins, e.g., transcription factors and proteins involved in pentose phosphate cycle, secretion pathways, signal transduction pathways, pH/stress response, and post-translational modifications play a role in enhancing production of active proteins and improving hydrolysis activity.

In the context of this invention, the proteins involved in degrading cellulosic biomass, e.g., a glycoside hydrolase or accessory enzyme, either directly are referred to as biomass degradation polypeptides. A protein that enhances production of proteins from a cell, e.g., by increasing secretions of a protein production, increasing expression of a protein, or inhibiting expression of a protein that suppresses secretion or expression is referred to as a “protein productivity” polypeptide.

### SUMMARY OF THE INVENTION

In one aspect, the invention provides a method of producing a biomass degradation polypeptide or a protein productivity polypeptide. The method involves culturing a cell comprising a recombinant polynucleotide sequence that encodes a Myceliophthora thermophila polypeptide comprising an amino acid sequence selected from the protein sequences of Tables 1, 2, 3, or 4. In some embodiments, the polypeptide comprises an amino acid sequence selected from the protein sequences of Table 3 or Table 4. In some embodiments, the recombinant polynucleotide sequence is operably linked to a promoter, or the polynucleotide sequence is present in multiple copies operably linked to a promoter, under conditions in which the polypeptide is produced. In some embodiments, the promoter is a heterologous promoter. In some embodiments, the polypeptide comprises a fragment that is less than the full-length of a polypeptide identified in Tables 1, 2, 3, or 4. In some embodiments, the polypeptide consists of an amino acid sequence selected from the polypeptide sequences disclosed in Tables 1, 2, 3, or 4. Optionally, a polynucleotide sequence encoding a polypeptide of the invention has a nucleotide sequence selected from the cDNA sequences disclosed in Tables 1, 2, 3, or 4. In some embodiments, the polynucleotide has a nucleotide sequence selected from the cDNA sequences disclosed in Table 3 or Table 4.

Also contemplated is a method of converting biomass substrates to soluble sugars by combining a recombinant biomass degradation polypeptide made according to the invention with biomass substrates under conditions suitable for the production of the soluble sugar. In some embodiments, the method includes the step of recovering the biomass degradation polypeptide from the medium in which the cell is cultured. In one aspect a composition comprising a recombinant biomass degradation peptide of the invention is provided.

In one aspect, the invention provides a method for producing soluble sugars from biomass by contacting the biomass with a recombinant cell comprising a recombinant polynucleotide sequence that encodes a biomass degradation enzyme having an amino acid sequence selected from the protein sequences of Tables 1-4, typically selected from the protein sequences of Table 1 or Table 3, where the polynucleotide sequence is operably linked to a promoter, under conditions in which the enzyme is expressed and secreted by the cell and said cellulosic biomass is enzymatically converted using the biomass degradation enzyme to a degradation product that produces soluble sugar. In some embodiments, the promoter is a heterologous promoter. In some embodiments, the polynucleotide encodes a polypeptide comprising a sequence set forth in Column 4 of Table 1 or Table 3. In some embodiments, the polynucleotide encodes a polypeptide comprising a sequence set forth in Column 5 of Table 1 or Table 3 linked to a heterologous signal peptide. In some embodiments, multiple copies of the polynucleotide sequence may be operably linked to a promoter. In some embodiments, the polypeptide comprises a fragment that is less than the full-length of a polypeptide identified in Tables 1, 2, 3, or 4. Optionally, the polynucleotide encoding the biomass degradation enzyme has a nucleic acid sequence selected from the cDNA sequences identified in Table 1 or Table 3.

In a further aspect, the invention provides a method of enhancing protein production of a host cell, the method comprising genetically modifying a host cell to express a protein productivity polypeptide if Tables 1, 2, 3, or 4. In some embodiment, the polypeptide has the activity designation “42” in Column 2 of Tables 1, 2, 3, or 4.

In some embodiments of the methods of the invention, the cell in which a polypeptide of Tables 1, 2, 3, or 4 is expressed is a fungal cell. In some embodiments, the cell is a Myceliophthora thermophila cell and/or the heterologous promoter is a Myceliophthora thermophila promoter.

In one aspect, the invention provides a recombinant host cell comprising a recombinant polynucleotide sequence encoding a polypeptide comprising an amino acid sequence selected from the polypeptide sequences identified in Table 1, Table 2, Table 3, and Table 4, operably linked to a promoter, optionally a heterologous promoter. In some embodiments, the polypeptide comprises a fragment that is less than the full-length of a polypeptide identified in Tables 1, 2, 3, or 4. In some embodiments, the polypeptide consists of an amino acid sequence set forth in Tables 1, 2, 3, or 4. Optionally, the recombinant polynucleotide has a nucleic acid sequence selected from the cDNA sequences identified in Tables 1, 2, 3, or 4. In one embodiment, the recombinant host cell expresses at least one other recombinant polypeptide, e.g., a cellulase enzyme or other enzyme involved in degradation of cellulosic biomass.

In a further aspect, also contemplated is a method of converting a biomass substrate to a soluble sugar, by combining an expression product from a recombinant cell that expresses a polypeptide of Tables 1, 2, 3, or 4, with a biomass substrate under conditions suitable for the production of soluble sugar(s).

In a further aspect, the invention provides a composition comprising an enzyme having an amino acid sequence selected from the group of glycoside hydrolase amino acid sequences set forth in Tables 1, 2, 3, or 4 and a cellulase, wherein the amino acid sequence of the cellulase is different from the glycoside hydrolase biomass degradation enzyme selected from Tables 1, 2, 3, or 4. In some embodiments, the cellulase is derived from a filamentous fungal cell, e.g., a Trichoderma sp. or an Aspergillus sp.

In a further aspect, the invention provides a genetically modified host cell in which a gene encoding a polypeptide of Tables 1, 2, 3, or 4, is disrupted.

In a further aspect, the invention additionally provides an isolated polypeptide comprising an amino acid sequence of Tables 1, 2, 3, or 4. In some embodiments, the polypeptide is a glycohydrolase or carbohydrate esterase. In some embodiments, the enzyme is an arabinofuranosidase of the GH3, GH43, GH51, GH54, or GH62 family. In some embodiments, the enzyme is a xyloglucanase of the GH5, GH12, GH16, GH44, or GH74 family. In some embodiments, the enzyme is an alpha-glucuronidase of the GH67 or GH115 family. In some embodiments, the enzyme is a beta-xylosidase of the GH3, GH30, GH39, GH43, GH52, or GH54 family. In some embodiments, the enzyme is a beta-galactosidase of the GH2 or GH42 family. In some embodiments, the enzyme is an arabinofuranosidase/arabinase of the GH3, GH43, GH51, GH54, GH62, or GH93 family. In some embodiments, the enzyme is an endo-xylanase of the of the GH5, GH8, GH10, or GH11 family. In some embodiments, the enzyme is a xylanase of the GH5, GH8, GH10, or GH11 family. In some embodiments, the enzyme is a polygalacturonase of the GH28 family. In some embodiments, the enzyme is a beta-glucosidase of the GH1, GH3, GH9, or GH30 family. In some embodiments, the enzyme is a beta-1,3-glucanase of the GH5, GH12. GH16, GH17, GH55, GH64 or GH81 family. In some embodiments, the enzyme is an alpha-1,6-mannanase of the GH38, GH76, or GH92. In some embodiments, the enzyme is a rhamnoglacturonyl hydrolyase or the GH28 or GH105 family. In some embodiments, the enzyme is an alpha-amylase of the GH13 or GH57 family. In some embodiments, the enzyme is an alpha-glucosidase of the GH4, GH13, GH31 or GH63 family. In some embodiments, the enzyme is a glucoamylase of the GH15 family. In some embodiments, the enzyme is a glucanase of the GH5, GH6, GH7, GH8. GH9, GH12, GH13, GH14, GH15, GH16, GH17, GH30, GH44, GH48, GH49, GH51, GH55, GH57, GH64. GH71, GH74, or GH81 family. In some embodiments, the enzyme is an endo-glucanase of the GH5, GH6, GH7, GH8. GH9, GH12, GH44, GH45, or GH74 family. In some embodiments, enzyme is a fucosidase of the GH29 family. In some embodiments, the enzyme is an alpha-xylosidase of the GH31 family.

In a further aspect, the invention provides methods of using glycohydrolase enzymes. Examples of such methods are described, e.g., in U.S. Pat. No. 8,298,79, which is incorporated by reference. The invention thus provides a method employing a glycohydrolase for increasing yield of fermentable sugars in a reaction in which a cellulose-containing substrate undergoes saccharification by cellulase enzymes comprising an endoglucanase, a beta-glucosidase, and a cellobiohydrolase, where the method comprises conducting the reaction in the presence of a recombinant glycohydrolase polypeptide of Tables 1, 2, 3, or 4, or a biologically active fragment thereof, whereby the reaction results in a glucose yield that is at least 20% higher than a glucose yield obtained from a saccharification reaction under the same conditions in the absence of said glycohydrolase protein. In some embodiments, the cellulose containing substrate is obtained from wheat, wheat straw, sorghum, rice, barley, sugar cane straw, sugar cane bagasse, grasses, switchgrass, corn grain, corn cobs, corn fiber, corn stover, or a combination thereof.

The invention further provides a method of producing a biofuel comprising ethanol, the method comprising: a) contacting a cellulose containing substrate with: i) a plurality of cellulase enzymes comprising an endoglucanase, a beta-glucosidase, and a cellobiohydrolase; and ii) a recombinant glycohydrolase polypeptide of Tables 1, 2, 3, or 4, or a biologically active fragment thereof; under conditions whereby simple sugars are produced from the substrate; b) combining simple sugars produced in step (a) with fungal cells under conditions whereby fermentation occurs and ethanol is produced. In some embodiments, the cellulase enzymes are from M. thermophila. In some embodiments, the fungal cells are yeast cells. In some embodiments, the cellulose containing substrate is obtained from wheat, wheat straw, sorghum, rice, barley, sugar cane straw, sugar cane bagasse, grasses, switchgrass, corn grain, corn cobs, corn fiber, corn stover, or a combination thereof.

Additionally, the invention provides a method of producing fermentable sugars from a cellulose containing substrate, comprising combining the substrate with: a) an enzyme composition comprising one or more beta-glucosidases and one or more cellobiohydrolases; and b) a recombinant glycohydrolase polypeptide of Tables 1, 2, 3, or 4, or a biologically active fragment thereof; wherein the enzyme composition is substantially free of recombinant endoglucanase.

In additional aspects, the invention provides nucleic acids encoding a polypeptide of the invention and a host cell comprising such a nucleic acid. The host cell may be a prokaryotic or eukaryotic cell. In some embodiments, the host cell is a fungus cell, e.g., a yeast or a filamentous fungus. In some embodiments, the host cell is a filamentous fungus host cell, such as a Myceliophthora thermophila host cell.

### BRIEF DESCRIPTION OF THE TABLES

The SEQ ID NOs. shown in the Tables 1, 2, 3, and 4 refer to the nucleic acid and polypeptide sequences provided in the electronic sequence txt file filed herewith, which is incorporated by reference.

Tables 1 and 3: Column 1, Gene; Column 2. Activity No.; Column 3, SEQ ID of corresponding to the cDNA; Column 4, SEQ ID NO for the protein encoded by the cDNA of Column 2, including the signal peptide sequence; Column 5, SEQ ID NO for the protein encoded by the cDNA of column 3 without the signal peptide. The “Activity No.” shown in Column 2 refers to the activity number in Column 1 of Table 5.

Tables 2 and 4: Column 1, Gene; Column 2. Activity No.; Column 3, SEQ ID of corresponding to the cDNA; Column 4, SEQ ID NO for the protein encoded by the cDNA of Column 2. The “Activity No.” shown in Column 2 refers to the activity number in Column 1 of Table 5.

Table 5 shows the activity associated with the activity numbers listed in Tables 1 through 4. Table 5 includes Activity No. (Column 1); polypeptide activity (Column 2); and glycohydrolase (GH) family designations for GH enzymes; or Carbohydrate Esterase (CE) family designations for carbohydrate esterases (Column 3).

In the context of this invention, “a polynucleotide of” Tables 1, 2, 3, or 4 refers to a polynucleotide that comprises a nucleotide sequence of a sequence identifier shown in Column 3; “a polypeptide of” Tables 1, 2, 3, or 4 refers to a polypeptide that comprises an amino acid sequence of a sequence identifier shown in Column 4 and Column 5 (for Tables 1 and 3).

### DETAILED DESCRIPTION OF THE INVENTION

### I. Definitions

The following definitions are provided to assist the reader. Unless otherwise defined, all terms of art are intended to have the meanings commonly understood by those of skill in the molecular biology and microbiology arts. In some cases, terms with commonly understood meanings are defined herein for clarity and/or for ready reference, and the inclusion of such definitions herein should not necessarily be construed to represent a substantial difference over the definition of the term as generally understood in the art.

As used in the context of this invention, the term “cellulosic biomass”, “biomass” and “biomass substrate” are used interchangeably to refer to material that contains cellulose and/or lignocellulose. Lignocellulose is considered to be composed of cellulose (containing only glucose monomers); hemicellulose, which can contain sugar monomers other than glucose, including xylose, mannose, galactose, rhamnose, and arabinose; and lignin.

The term “biomass degradation enzyme” is used herein to refer to enzymes that participate in degradation of cellulosic biomass degradation, and includes enzymes that degrade cellulose, lignin and hemicellulose. The term thus encompasses cellulases, xylanases, carbohydrate esterases, lipases, and enzymes that break down lignin including oxidases, peroxidases, laccases, etc. Glycoside hydrolases (GHs) are noted in Tables 1, 2, 3, and 4 as a functional class. Other enzymes that are not glycoside hydrolases that participate in biomass degradation are also included in the invention. Such proteins may be referred to herein as “accessory proteins” or “accessory enzymes”.

A “biomass degradation product” as used herein can refer to an end product of cellulose and/or lignocellulose degradation such as a soluble sugar, or to a product that undergoes further enzymatic conversion to an end product such as a soluble sugar. For example, a laccase can participate in the breakdown of lignin and although the laccase does not directly generate a soluble sugar, treatment of a biomass with laccase can result in an increase in the cellulose that is available for degradation. Similarly, various esterases can remove phenolic and acetyl groups from lignocellulose to aid in the production of soluble sugars. In typical biomass degradation reactions, the cellulosic material is hydrolyzed to break down cellulose and/or hemicellulose to fermentable sugars, such as glucose, cellobiose, xylose, xylulose, arabinose, mannose, galactose, and/or soluble oligosaccharides.

“Glycoside hydrolases” (GHs), also referred to herein as “glycohydrolases”, (EC 3.2.1.) hydrolyze the glycosidic bond between two or more carbohydrates or between a carbohydrate and a non-carbohydrate moiety. The Carbohydrate-Active Enzymes database (CAZy) provides a continuously updated list of the glycoside hydrolase families. See, the web address “cazy.org/Glycoside-Hydrolases.html”.

“Carbohydrate esterases” (CEs) catalyze the de-O or de-N-acylation of substituted saccharides. The CAZy database provides a continuously updated list of carbohydrate esterase families. See, the web address “cazy.org/Carbohydrate-Esterases.html”.

The term “cellulase” refers to a category of enzymes capable of hydrolyzing cellulose (β-1,4-glucan or β-D-glucosidic linkages) to shorter oligosaccharides, cellobiose and/or glucose. Cellulases include 1,4-β-D-glucan glucanohydrolase (“endoglucanase” or “EG”); 1,4-β-D-glucan cellobiohydrolase (“exoglucanase”, “cellobiohydrolase”, or “CBH”); and β-D-glucoside-glucohydrolase (“β-glucosidase”, “cellobiase” or “BG”).

The term “β-glucosidase” or “cellobiase” used interchangeably herein means a β-D-glucoside glucohydrolase which catalyzes the hydrolysis of a sugar dimer, including but not limited to cellobiose, with the release of a corresponding sugar monomer. In one embodiment, αβ-glucosidase is a β-glucoside glucohydrolase of the classification E.C. 3.2.1.21 which catalyzes the hydrolysis of cellobiose to glucose. Some of the β-glucosidases have the ability to also hydrolyze β-D-galactosides, β-L-arabinosides and/or β-D-fucosides and further some β-glucosidases can act on α-1,4-substrates such as starch. β-glucosidase activity may be measured by methods well known in the art, including the assays described hereinbelow. β-glucosidases include, but are not limited to, enzymes classified in the GH1, GH3, GH9, and GH30 GH families,

The term “β-glucosidase polypeptide” refers herein to a polypeptide having β-glucosidase activity.

The term “exoglucanase”, “exo-cellobiohydrolase” or “CBH” refers to a group of cellulase enzymes classified as E.C. 3.2.1.91. These enzymes hydrolyze cellobiose from the reducing or non-reducing end of cellulose. Exo-cellobiohydrolases include, but are not limited to, enzymes classified in the GH5, GH6, GH7, GH9, and GH48 GH families.

The term “endoglucanase” or “EG” refers to a group of cellulase enzymes classified as E.C. 3.2.1.4. These enzymes hydrolyze internal β-1,4 glucosidic bonds of cellulose. Endoglucanases include, but are not limited to, enzymes classified in the GH5, GH6, GH7, GH8, GH9, GH12. GH44, GH45, GH48, GH51, GH61, and GH74 GH families.

The term “xylanase” refers to a group of enzymes classified as E.C. 3.2.1.8 that catalyze the endo-hydrolysis of 1,4-beta-D-xylosidic linkages in xylans. Xylanases include, but are not limited to, enzymes classified in the GH5, GH8, GH10, and GH11 GH families.

The term “xylosidase” refers to a group of enzymes classified as E.C. 3.2.1.37 that catalyze the exo-hydrolysis of short beta (1⇄4)-xylooligosaccharides, to remove successive D-xylose residues from the non-reducing termini. Xylosidases include, but are not limited to, enzymes classified in the GH3, GH30, GH39, GH43, GH52, and GH54 GH families.

The term “arabinofuranosidase” refers to a group of enzymes classified as E.C. 3.2.1.55 that catalyze the hydrolysis of terminal non-reducing alpha-L-arabinofuranoside residues in alpha-L-arabinosides. The enzyme activity acts on alpha-L-arabinofuranosides, alpha-L-arabinans containing (1,3)- and/or (1,5)-linkages, arabinoxylans, and arabinogalactans. Arabinofuranosidases include, but are not limited to, enzymes classified in the GH3, GH43, GH51, GH54, and GH62 GH families.

The term “biomass degradation enzyme activity” encompasses glycoside hydrolase enzyme activity, e.g., that hydrolyzes glycosidic bonds of cellulose, e.g., exoglucanase activity (CBH), endoglucanase (EG) activity and/or β-glucosidase activity, as well as the enzymatic activity of accessory enzymes such as carbohydrate esterases, e.g., aryl esterases, including feruloyl and coumaroyl esterases, acetyl esterases, laccases, dehydrogenases, oxidases, peroxidases, and the like.

The term “protein production polypeptide” encompasses proteins that play a role in controlling the amount of active protein, i.e., properly folded and modified and thus, functional, protein, produced by a cell. Such polypeptides include transcription factors, and polypeptides involved in the pentose phosphate cycle, secretion pathways, signal transduction pathways, pH/stress response, and post-translational modification pathways. In some embodiments, a protein production polypeptide of the invention has an activity designated as “42” in Column 2 of Table 1, Table, 2, Table 3, or Table 4.

The term “biomass degradation polynucleotide” refers to a polynucleotide encoding a polypeptide of the invention that play a role in degrading a cellulosic biomass, e.g., a biomass degradation enzyme of Tables 1, 2, 3, or 4.

A “protein production polynucleotide” refers to a polynucleotide encoding a polypeptide of the invention e.g., a protein having an activity designation “42” in Column 2 of Tables 1, 2, 3, or 4, that plays a role in the production of active proteins by a cell.

As used herein, the term “isolated” refers to a nucleic acid, polynucleotide, polypeptide, protein, or other component that is partially or completely separated from components with which it is normally associated (other proteins, nucleic acids, cells, synthetic reagents, etc.).

The term “wildtype” as applied to a polypeptide (protein) means a polypeptide (protein) expressed by a naturally occurring microorganism such as bacteria or filamentous fungus. As applied to a microorganism, the term “wildtype” refers to the native, naturally occurring non-recombinant micro-organism.

A nucleic acid (such as a polynucleotide), and a polypeptide is “recombinant” when it is artificial or engineered. A cell is recombinant when it contains an artificial or engineered protein or nucleic acid or is derived from a recombinant parent cell. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

The term “culturing” or “cultivation” refers to growing a population of microbial cells under suitable conditions in a liquid or solid medium. In some embodiments, culturing refers to fermentative bioconversion of a cellulosic substrate to an end-product.

The term “contacting” refers to the placing of a respective enzyme in sufficiently close proximity to a respective substrate to enable the enzyme to convert the substrate to a product. Those skilled in the art will recognize that mixing solution of the enzyme with the respective substrate will effect contacting.

As used herein the term “transformed” or “transformation” used in reference to a cell means a cell has a non-native nucleic acid sequence integrated into its genome or as an episomal plasmid that is maintained through multiple generations.

The term “introduced” in the context of inserting a nucleic acid sequence into a cell means transfected, transduced or transformed (collectively “transformed”) and prokaryotic cell wherein the nucleic acid is incorporated into the genome of the cell.

As used herein, “C1” refers to Myceliophthora thermophila, including a fungal strain that was initially as described by Garg as Chrysosporium lucknowense (Garg, A., 1966, “An addition to the genus Chrysosporium corda” Mycopathologia 30: 3-4). “Myceliophthora thermophila” in the context of the present invention, includes various strains described in U.S. Pat. Nos. 6,015,707, 5,811,381 6,573,086, 8,236,551 and 8,309,328; US Pat. Pub. Nos. 2007/0238155, US 2008/0194005, US 2009/0099079; International Pat. Pub. Nos., WO 2008/073914 and WO 98/15633, and include, without limitation, Chrysosporium lucknowense Garg 27K, VKM-F 3500 D (Accession No. VKM F-3500-D), C1 strain UV13-6 (Accession No. VKM F-3632 D), C1 strain NG7C-19 (Accession No. VKM F-3633 D), and C1 strain UV18-25 (VKM F-3631 D), all of which have been deposited at the All-Russian Collection of Microorganisms of Russian Academy of Sciences (VKM), Bakhurhina St. 8, Moscow, Russia, 113184, and any derivatives thereof. Exemplary C1 strains include modified organisms in which one or more endogenous genes or sequences has been deleted or modified and/or one or more heterologous genes or sequences has been introduced, such as UV18#100.f (CBS Accession No. 122188). Derivatives include UV18#100.f Δalp1, UV18#100.f Δpyr5 Δalp1, UV18#100.f Δalp1 Δpep4 Δalp2, UV18#100.f Δpyr5 Δalp1 Δpep4 Δalp2 and UV18#100.f Δpyr4 Δpyr5 Δalp 1 Δpep4 Δalp2, as described in WO2008073914, incorporated herein by reference.

The term “operably linked” refers herein to a configuration in which a control sequence is appropriately placed at a position relative to the coding sequence of the DNA sequence such that the control sequence influences the expression of RNA encoding a polypeptide.

When used herein, the term “coding sequence” is intended to cover a nucleotide sequence that directly specifies the amino acid sequence of its protein product. The boundaries of the coding sequence are generally determined by an open reading frame, which usually begins with the ATG start codon.

A promoter or other nucleic acid control sequence is “heterologous”, when it is operably linked to a sequence encoding a protein sequence with which the promoter is not associated in nature. For example, in a recombinant construct in which a Myceliophthora thermophila Cbh1a promoter is operably linked to a protein coding sequence other than the Myceliophthora thermophila Cbh1a gene to which the promoter is naturally linked, the promoter is heterologous. For example, in a construct comprising a Myceliophthora thermophila Cbh1a promoter operably linked to a Myceliophthora thermophila nucleic acid encoding a biomass degradation enzyme of Tables 1, 2, 3, or 4, the promoter is heterologous. Similarly, a polypeptide sequence such as a secretion signal sequence, is “heterologous” to a polypeptide sequence when it is linked to a polypeptide sequence that it is not associated with in nature.

As used herein, the term “expression” includes any step involved in the production of the polypeptide including, but not limited to, transcription, post-transcriptional modification, translation, post-translational modification, and secretion.

The term “expression vector” refers herein to a DNA molecule, linear or circular, that comprises a segment encoding a polypeptide of the invention, and which is operably linked to additional segments that provide for its transcription.

A polypeptide of the invention is “active” when it has a biomass degradation activity or increase protein productivity. Thus, a polypeptide of the invention may have a glycoside hydrolase activity, or another enzymatic activity shown in Table 5.

The term “pre-protein” refers to a secreted protein with an amino-terminal signal peptide region attached. The signal peptide is cleaved from the pre-protein by a signal peptidase prior to secretion to result in the “mature” or “secreted” protein.

As used herein, a “start codon” is the ATG codon that encodes the first amino acid residue (methionine) of a protein.

The terms “peptide,” “polypeptide,” and “protein” are used interchangeably herein to refer to a polymer of amino acid residues.

The term “amino acid” refers to naturally occurring and synthetic amino acids, as well as amino acid analogs. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group. e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

### II. Introduction

The fungus Myceliophthora thermophila produces a variety of enzymes that act in concert to catalyze decrystallization and hydrolysis of cellulose to yield soluble sugars. The present invention is based on the discovery and characterization of Myceliophthora thermophila genes encoding biomass degradation polypeptides that facilitate biomass degradation and the discovery and characterization of Myceliophthora thermophila genes that enhance protein productivity of cells recombinantly engineered to have modified expression of the protein productivity genes.

The biomass degradation polypeptides of the invention, and polynucleotides encoding them, may be used in a variety of applications for degrading cellulosic biomass, such as those described hereinbelow. For simplicity, and as will be apparent from context, references to a “biomass degradation polypeptide” and the like may be used to refer both to a secreted mature form of the polypeptide and to the pre-protein form.

A protein productivity polypeptide, and polynucleotides encoding them, may be used in a variety of applications for enhancing protein production of a cell. References to a “protein productivity polypeptide” may be used to refer to both a mature form of a polypeptide and to a pre-protein form.

In various embodiments of the invention, a recombinant nucleic acid sequence is operably linked to a promoter. In one embodiment, a nucleic acid sequence encoding a polypeptide comprising an amino acid sequence of Tables 1, 2, 3, or 4 is operably linked to a promoter not associated with the polypeptide in nature (i.e., a heterologous promoter), to, for example, improve expression efficiency of a biomass degradation polypeptide or protein productivity polypeptide when expressed in a host cell. In one embodiment the host cell is a fungus, such as a filamentous fungus. In one embodiment the host cell is a Myceliophthora thermophila cell. In one embodiment the host cell is a Myceliophthora thermophila cell and the promoter is a heterologous Myceliophthora thermophila promoter.

A polypeptide expression system comprising one or more polypeptides of Tables 1, 2, 3, or 4 is particularly useful for degradation of cellulosic biomass to obtain soluble carbohydrates from the cellulosic biomass. In one aspect the invention relates to a method of producing a soluble sugar, e.g., glucose, xylose, etc., by contacting a composition comprising cellulosic biomass with a recombinantly expressed polypeptide, e.g., a glycohydrolase or accessory enzyme, of Tables 1, 2, 3, or 4, e.g., a glycohydrolase of Tables 1, 2, 3, or 4, under conditions in which the biomass is enzymatically degraded. In some embodiments, the cellulosic biomass is contacted with one or more accessory enzymes of Tables 1, 2, 3, or 4. Purified or partially purified recombinant biomass degradation enzymes may be contacted with the cellulosic biomass. In one aspect of the present invention, “contacting” comprises culturing a recombinant host cell in a medium that contains biomass produced from a cellulosic biomass feedstock, where the recombinant cell comprises a sequence encoding a biomass degradation polypeptide of Tables 1, 2, 3, or 4 operably linked to a heterologous promoter or to a homologous promoter when the sequence is present in multiple copies per cell.

In some embodiments, a polypeptide of the invention comprises an active fragment, e.g., a fragment that retains catalytic activity or activity of another domain, such as binding, of a polypeptide having an amino acid sequence set forth in Tables 1, 2, 3, or 4.

In another aspect of the invention, a heterologous Myceliophthora thermophila signal peptide may be fused to the amino terminus of a polypeptide of column 5 in Table 1 and Table 3; or a polypeptide of Table 2 or Table 4 to improve post-translational modification, secretion, folding, stability, or other properties of the polypeptide when expressed in a host cell. e.g., a fungal cell such as a Myceliophthora thermophila cell.

In some embodiments, a biomass degradation enzyme of the invention has an amino acid sequence identified in any of Tables 1-4 and is a glycohydrolase. In some embodiments, the enzyme is an arabinofuranosidase of the GH3, GH43, GH51, GH54, or GH62 family. In some embodiments, the enzyme is a xyloglucanase of the GH5, GH12, GH16, GH44, or GH74 family. In some embodiments, the enzyme is an alpha-glucuronidase of the GH67 or GH115 family. In some embodiments, the enzyme is a beta-xylosidase of the GH3, GH30, GH39, GH43, GH52, or GH54 family. In some embodiments, the enzyme is a beta-galactosidase of the GH2 or GH42 family. In some embodiments, the enzyme is an arabinofuranosidase/arabinase of the GH3, GH43, GH51, GH54, GH62, or GH93 family. In some embodiments, the enzyme is an endo-xylanase of the of the GH5, GH8, GH10, or GH11 family. In some embodiments, the enzyme is a xylanase of the GH5. GH8. GH10, or GH11 family. In some embodiments, the enzyme is a polygalacturonase of the GH28 family. In some embodiments, the enzyme is a beta-glucosidase of the GH1, GH3, GH9, or GH30 family. In some embodiments, the enzyme is a beta-1,3-glucanase of the GH5. GH12, GH16, GH17, GH55, GH64 or GH81 family. In some embodiments, the enzyme is an alpha-1,6-mannanase of the GH38, GH76, or GH92. In some embodiments, the enzyme is a rhamnoglacturonyl hydrolyase or the GH28 or GH105 family. In some embodiments, the enzyme is an alpha-amylase of the GH13 or GH57 family. In some embodiments, the enzyme is an alpha-glucosidase of the GH4, GH13, GH31 or GH63 family. In some embodiments, the enzyme is a glucoamylase of the GH15 family. In some embodiments, the enzyme is a glucanase of the GH5, GH6, GH7, GH8, GH9, GH12, GH13, GH14, GH15, GH16, GH17, GH30, GH44, GH48, GH49, GH51, GH55, GH57, GH64, GH71, GH74, or GH81 family. In some embodiments, the enzyme is an endo-glucanase of the GH5, GH6, GH7, GH8, GH9, GH12, GH44, GH45, or GH74 family. In some embodiments, enzyme is a fucosidase of the GH29 family. In some embodiments, the enzyme is an alpha-xylosidase of the GH31 family.

In some embodiments, a polypeptide of the invention has an amino acid sequence identified in any of Tables 1-4 and is an accessory enzyme. In some embodiments, the biomass degradation enzyme is an acetyl esterase, acetyl xylan esterase, ferulic acid esterase, glucuronyl esterase, laccase, cutinase, protease, oxidase, peroxidase, reductase, pectin acetyl esterase or rhamnogalactouronan acetyl esterase, or dehydrogenase.

In some embodiments, a polypeptide of the invention has an amino acid sequence identified in any of Tables 1-4 and is a protein productivity polypeptide. In some embodiments, the protein is a transcription factor; a protein in the pentose phosphate cycle, a protein in a signal transduction pathway, a protein in the secretion pathways, a pH/stress response protein, or a protein that plays a role in post-translational modification. In some embodiments, the protein has the designation “42” in Column 2 of Tables 1, 2, 3, or 4.

Various aspects of the invention are described in the following sections.

### III. Properties of Myceliophthora Thermophila Polypeptides of the Invention

In one aspect, the invention provides a method for expressing a Myceliophthora thermophila polypeptide of the invention where the method involves culturing a host cell comprising a vector comprising a nucleic acid sequence encoding a polypeptide sequence of Tables 1, 2, 3, or 4 operably linked to a heterologous promoter, under conditions in which the polypeptide or an active fragment thereof is expressed. In some embodiments, the expressed protein comprises a signal peptide that is removed in the secretion process. In some embodiments, the nucleic acid sequence is a nucleic acid sequence of Tables 1, 2, 3, or 4.

In some embodiments the polypeptide of Tables 1, 2, 3, or 4 includes additional sequences that do not alter the activity of the encoded polypeptide. For example, the polypeptide may be linked to an epitope tag or to other sequence useful in purification. In some embodiments, a polypeptide of the invention, or a functional domain thereof may be linked to heterologous amino acid sequence in a fusion protein. For example, a catalytic domain of a polypeptide of Table 1, Table, Table 3, or Table 4 may be linked to a domain, e.g., a binding domain, from a heterologous polypeptide.

### Signal Peptide

In some embodiments, polypeptides of the invention are secreted from the host cell in which they are expressed as a pre-protein including a signal peptide, i.e., an amino acid sequence linked to the amino terminus of a polypeptide that directs the encoded polypeptide into the cell secretory pathway. In one embodiment, the signal peptide is an endogenous signal peptide of a polypeptide sequence of Column 5 Table 1 or Column 5 Table 3. In other embodiments, a signal peptide from another Myceliophthora thermophila secreted protein is used.

Other signal peptides may be used, depending on the host cell and other factors. Effective signal peptide coding regions for filamentous fungal host cells include but are not limited to the signal peptide coding regions obtained from Aspergillus oryzae TAKA amylase, Aspergillus niger neutral amylase, Aspergillus niger glucoamylase. Rhizomucor miehei aspartic proteinase, Humicola insolens cellulase, Humicola lanuginosa lipase, and T. reesei cellobiohydrolase II. For example, a polypeptide sequence of the invention may be used with a variety of filamentous fungal signal peptides known in the art. Useful signal peptides for yeast host cells also include those from the genes for Saccharomyces cerevisiae alpha-factor and Saccharomyces cerevisiae invertase. Still other useful signal peptide coding regions are described by Romanos et al., 1992, Yeast 8:423-488. Effective signal peptide coding regions for bacterial host cells are the signal peptide coding regions obtained from the genes for Bacillus NCIB 11837 maltogenic amylase, Bacillus stearothermophilus alpha-amylase. Bacillus lichenformis subtilisin, Bacillus licheniformis β-lactamase, Bacillus stearothermophilus neutral proteases (nprT, nprS, nprM), and Bacillus subtilis prsA. Further signal peptides are described by Simonen and Palva, 1993, Microbiol Rev 57: 109-137. Variants of these signal peptides and other signal peptides are also suitable.

In a further aspect, the invention provides a biologically active variant of a polypeptide having an amino acid sequence of Tables 1, 2, 3, or 4, nucleic acids encoding such variant polypeptides, methods of producing such variant polypeptides, and methods of using the variant polypeptides to degrade cellulosic biomass or to increase protein productivity.

The term “variant” refers to a polypeptide having substitutions, additions, or deletions at one or more positions relative to a wild type polypeptide. The term encompasses functional (or “biologically active”) fragments of a polypeptide. In one embodiment, a “variant” comprises at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a specified reference sequence. Variants include homologs (i.e., which may be endogenous to a related microbial organism) and polymorphic variants. Homologs and polymorphic variants can be identified based on sequence identity and similar biological (e.g., enzymatic) activity.

As used herein, a “functional fragment” refers to a polypeptide that has an amino-terminal deletion and/or carboxyl-terminal deletion and/or internal deletion, but where the remaining amino acid sequence is identical or substantially identical to the corresponding positions in the sequence to which it is being compared (e.g., a full-length polypeptide sequence) and that retains substantially all of the activity of the full-length polypeptide, or a functional domain of the full-length polypeptide. In various embodiments, a functional fragment of a full-length wild-type polypeptide comprises at least about 70%, at least about 75%, at least about 80%, at least about 90%, at least about 91%, at least about 92%, at least about 93%, at least about 94%, at least about 95%, at least about 96%, at least about 97%, at least about 98%, or at least about 99% identity to the wild-type or reference amino acid sequence. In certain embodiments, a functional fragment comprises about 75%, about 80%, about 85%, at about 90%, about 91%, about 92%, about 93%, about 94%, about 95%, about 96%, about 97%, about 98%, or about 99% of the amino acid sequence of a full-length polypeptide.

The term “substantial identity” or “substantially identical” refers to in the context of two nucleic acid or polypeptide sequences, refers to a sequence that has at least 70% identity to a reference sequence. Percent identity can be any integer from 70% to 100%. Two nucleic acid or polypeptide sequences that have 100% sequence identity are said to be “identical.” A nucleic acid or polypeptide sequence are said to have “substantial sequence identity” to a reference sequence when the sequences have at least about 70%, at least about 75%, at least 80%, at least 85%, at least 90%, at least 91%, at least 92%, at least 93%, at least 94%, at least 95%, at least 96%, at least 97%, at least 98%, or at least 99% or greater sequence identity as determined using known methods, such as BLAST using standard parameters as described above.

### Polypeptide Activity

The activity of a polypeptide of the invention, e.g., to evaluate activity of a variant, evaluate an expression system, assess activity levels in an enzyme mixture comprising the enzyme, etc., can be determined by methods well known in the art for each of the various polypeptides of Tables 1, 2, 3, or 4. For example, esterase activity can be determined by measuring the ability of an enzyme to hydrolyze an ester. Glycoside hydrolase activity can be determined using known assays to measure the hydrolysis of glyosidic linkages. Enzymatic activity of oxidases and oxidoreductases can be assessed using techniques to measure oxidation of known substrates. Activity of protein productivity polypeptides can be assessed using known assays such as a BCA assay that measures protein concentrations and/or SDS-PAGE that measure secreted proteins. Assay for measuring activity of a polypeptide of Tables 1, 2, 3, or 4 are known to those of ordinary skill, and are described in the scientific anc patent literature. Illustrative polypeptide activity assays are further detailed below. One of skill understands that alternative assays are known and can be used instead of the illustrative assays.

### Alpha-Arabinofuranosidase Enzymatic Activity

Alpha-arabinofuranosidase activity can be measured using assays well known in the art. For example, enzymatic activity of an alpha-arabinofuranosidase can be measured by measuring the release of p-nitrophenol by the action of alpha-arabinofuranosidase on p-nitrophenyl alpha-L-arabinofuranoside (PNPA). One alpha-arabinofuranosidase unit of activity is the amount of enzyme that liberates 1 micromole of p-nitrophenol in one minute at 37° C. and pH 5.0. An illustrative assay is as follows: PNPA is used as the assay substrate. PNPA is dissolved in distilled water and 0.1 M acetate buffer (pH 5.0) to obtain a 1 mM stock solution. A stop reagent (0.25 M sodium carbonate solution) is used to terminate the enzymatic reaction. For the enzyme sample, 0.10 mL of 1 mM PNPA stock solution is mixed with 0.01 mL of the enzyme sample and incubated at 37° C. for 90 minutes. After 90 minutes of incubation, 0.1 mL of 0.25 M sodium carbonate solution is added and the absorbance at 405 nm (A₄₀₅) is then measured in microtiter plates as A_{S}. Absorbance is also measure for a substrate blank A_{SB}. Activity is calculated as follows:

$$Activity ( IU \text{/} ml ) = \frac{Δ {A}_{405} * DF * 21 * 1.33}{13.700 * RT}$$

where ΔA₄₀₅=A_{S}−A_{SB}, DF is the enzyme dilution factor, 21 is the dilution of 10 ul enzyme solution in 210 ul reaction volume, 1.33 is the conversion factor of microtiter plates to cuvettes, 13.700 is the extinction coefficient 13700 M⁻¹ cm⁻¹ of p-nitrophenol released corrected for mol/L to umol/mL, and RT is the reaction time in minutes.

This assay can be used to test the activity of enzymes such as, but not limited to, GH3, GH43, GH51, GH54, and GH62 enzymes. Thus, for example, this assay can be used to test the activity of an enzyme such as, but not limited to, an enzyme designated with an activity of “3” in column 2 of Tables 1, 2, 3, or 4.

Ability of Enzymes of the Present Invention to Remove the α-L-Arabinofuranosyl Residues from Substituted Xylose Residues

The ability of enzymes of the present invention to remove the α-L-arabinofuranosyl residues from substituted xylose residues can be assayed using known assays. An illustrative assay is as follows. For the complete degradation of arabinoxylans to arabinose and xylose, several enzyme activities are needed, including endo-xylanases and arabinofuranosidases. The arabinoxylan molecule from wheat is highly substituted with arabinosyl residues. These can be substituted either to the C2 or the C3 position of the xylosyl residue (single substitution), or both to the C2 and C3 position of the xylose (double substitution). An arabinofuranosidase from Bifidobacterium adolescentis (AXHd₃) has previously been isolated which is able to liberate the arabinosyl residue substituted to the C₃ position of a double substituted xylose. Most of the known arabinofuranosidases are only active towards single arabinosyl substituted xyloses. Single and double substituted oligosaccharides are prepared by incubating wheat arabinoxylan (WAX; 10 mg/mL; Megazyme, Bray, Ireland) in 50 mM acetate buffer pH 5 with 0.3 mg Pentopan Mono (mono component endo-1,4-xylanase, an enzyme from Thermomyces lanuginosus produced in Aspergillus oryzae; Sigma. St. Louis, USA) for 16 hours at 30° C. The reaction is stopped by heating the samples at 100° C. for 10 minutes. The samples are centrifuged for 5 minutes at 3100×g. The supernatant is used for further experiments. Degradation of the arabinoxylan is followed by analysis of the formed reducing sugars and High Performance Anion Exchange Chromatography (HPAEC).

Double substituted arabinoxylan oligosaccharides are prepared by incubation of 800 ul of the supernatant described above with 0.18 mg of the arabinofuranosidase Abfl (Abfl is arabinofuranosidase from M. thermophila with activity towards single arabinose substituted xylose residues and is disclosed in U.S. application Ser. No. 11/833,133, filed Aug. 2, 2007) in 50 mM acetate buffer pH 5 for 20 hours at 30° C. The reaction is stopped by heating the samples at 100° C. for 10 minutes. The samples are centrifuged for 5 minutes at 10,000×g, and the supernatant is used for further experiments. Degradation of the arabinoxylan is followed by analysis of the formed reducing sugars and HPAEC. The enzyme (25 gig total protein) is incubated with single and double substituted arabinoxylan oligosaccharides (100 supernatant of Pentopan Mono treated WAX) in 50 mM acetate buffer at 30° C. during 20 hours. The reaction is stopped by heating the samples at 100° C. for 10 minutes. The samples are centrifuged for 5 minutes at 10,000×g. Degradation of the arabinoxylan is followed by HPAEC analysis. The enzyme (25 μg total protein) from B. adolescentis (10 μl, 0.02 U; Megazyme, Bray, Ireland) is incubated with double substituted arabinoxylan oligosaccharides (125 μl supernatant of Pentopan Mono and Abfl treated WAX) in 50 mM acetate buffer at 35° C. during 24 hours. The reaction is stopped by heating the samples at 100° C. for 10 minutes. The samples are centrifuged for 5 minutes at 10,000×g. Degradation of the arabinoxylan is followed by HPAEC analysis.

The amount of reducing sugars is measured using a DNS (3,5-dinitro salicylic acid) assay. 0.5 mL of DNS reagent (3,5-dinitrosalicylic acid and sodium potassium tartrate dissolved in dilute sodium hydroxide) is added to the sample (50 ul), containing 0-5 mg/ml reducing sugar. The reaction mixture is heated at 100° C. for 5 minutes and rapidly cooled in ice to room temperature. The absorbance at 570 nm is measured. Glucose is used as a standard.

Single and double substituted arabinoxylan oligosaccharides are prepared by xylanase treatment as described above. Oligosaccharides are identified using known techniques. In addition to non-substituted oligosaccharides (xylobiose (X₂), xylotriose (X₃), xylotetraose (X₄)), single (X₃A, X₂A) and double substituted (X₄A₂, X₃A₂) oligosaccharides are also present after xylanase treatment. The activity towards this mixture of arabinoxylan oligosaccharides is then determined using the assays described above.

To generate samples with only double substituted oligosaccharides present, the single substituted oligosaccharides is removed from the xylanase-treated WAX mixture by the enzyme Abfl as described above. To generate samples with only single substituted oligosaccharides present, the double substituted oligosaccharides are removed from the xylanase-treated WAX mixture by the enzyme AXHd+ as described above. Samples containing only single substituted oligosaccharides or double substituted oligosaccharides are treated with the target enzyme or AXHd3 from B. adolescentis as a reference enzyme as described above.

This assay can be used to test the activity of enzymes such as, but not limited to, GH3, GH43, GH51, GH54, and GH62 enzymes. Thus, for example, this assay can be used to test the activity of an enzyme such as, but not limited to, an enzyme designated with an activity of “4” in column 2 of Tables 1, 2, 3, or 4.

### Xyloglucanase Activity

Xyloglucanase activity can be measured using assays well known in the art. The following is an illustrative assay. Activity is demonstrated by using xyloglucan as substrate and a reducing sugars assay (PAHBAH) as detection method. The values are compared to a standard, which is prepared using a commercial cellulase preparation from Aspergillus niger. A cellulase standard contains 2 units of cellulase per ml of 0.2 M HAc/NaOH, pH 5 is used to prepare a standard series. A working reagent containing PAHBAH is prepared (10 g of p-hydroxy benzoic acid hydrazide (PAHBAH) is suspended in 60 mL water. 10 mL of concentrated HCl is added and the volume adjusted to 200 ml. Reagent B is 24.0 g of trisodium citrated dissolved in 500 ml of water. 2.2 g of calcium chloride and 40 mg of NaOH are added and the volume adjusted to 2 L. with water. Working reagent: 10 ml Reagent A added to 90 ml of Reagent B.

The assay is conducted in micro titer plate format. Each well contains 50 ul of xyloglucan substrate (0.25% (w/v) tamarind xyloglucan in water), 30 ul of 0.2 M HAc/NaOH pH 5, 20 ul xyloglucanase sample or cellulase standard sample. These are incubated at 37° C. for 2 hours. After incubation 25 ul of each well are mixed with 125 ul working reagent. These solutions are heated at 95° C. for 5 minutes. After cooling down, the samples are analyzed by measuring the absorbance at 410 nm (A₄₁₀) as A_{S} (enzyme sample). Enzyme activities are determined using a standard curve. A substrate blank is also prepared and absorbance at 410 nm (A₄₁₀), A_{SB}, is measured.

Activity is calculated as follows: xyloglucanase activity is determined by reference to a standard curve of the cellulase standard solution.

Activity (IU/ml)=ΔA₄₁₀/SC*DF

where ΔA₄₁₀=A_{S} (enzyme sample)−A_{SB} (substrate blank), SC is the slope of the standard curve and DF is the enzyme dilution factor.

This assay can be used to test the activity of enzymes such as, but not limited to, GH5, GH12, GH16, GH44, and GH74 enzymes. Thus, for example, this assay can be used to test the activity of an enzyme such as, but not limited to, an enzyme designated with an activity of “5” in column 2 of Tables 1, 2, 3, or 4.

### Alpha-Glucuronidase Activity

Activity of an alpha-glucuronidase enzyme can be determined using known assays. The following illustrates an assay to measure the alpha-glucuronidase activity towards arabinoxylan oligosaccharides from Eucalyptus wood. This assay measures the release of glucuronic acid by the action of the α-glucuronidase on the arabinoxylan oligosaccharides.

Acetylated, 4-O-MeGlcA substituted xylo-oligosaccharides with 2-4 xylose residues or 4-10 xylose residues from Eucalyptus wood (EW-XOS) are prepared. One mg of xylo-oligosaccharides is dissolved in 1 mL distilled water. 4-o-MeGlcA is purified using known methods. Aldo-biuronic acid (X₁G), aldo-triuronic acid (X₂G), and aldo-tetrauronic acid (X₃G) are obtained from Megazyme. To remove the acetyl groups in the XOS, either for reference or for substrates, 1 mg of substrate is dissolved in 120 ul water and 120 ul 0.1 M NaOH. After overnight incubation at 4° C., the pH of the samples is checked. A pH above 9.0 indicates that the saponification reaction is complete. 120 ul of 0.1 M acetic acid and 40 ul of 0.2 M Sodium acetate, pH 5.0 are added. The substrate concentration is 2.5 mg/mL in 50 mM sodium acetate buffer, pH 5.0.

1 mL of xylo-oligosaccharides stock solution is mixed with 0.68 μg of the enzyme sample and incubated at 35° C. for 24 hours. The reaction is stopped by heating the samples for 10 minutes at 100° C. The release of 4-O-methyl glucuronic acid and formation of new (arabino)xylan oligosaccharides are analyzed by High Performance Anion Exchange Chromatography and capillary electrophoresis. A substrate blank is also prepared using an arabinoxylan oligosaccharides stock solution.

HPAEC is performed using a Dionex HPLC system equipped with a Dionex CarboPac PA-1 (2 mm ID×250 mm) column in combination with a CarboPac PA guard column (1 mm ID×25 mm) and a Dionex EDet1 PAD-detector (Dionex Co., Sunnyvale). A flow rate of 0.3 mL/min is used with the following gradient of sodium acetate in 0.1 M NaOH: 0-50 min, 0-500 mM. Each elution is followed by a washing step of 5 min using 1 M sodium acetate in 0.1 M NaOH and an equilibration step of 15 min using 0.1 M NaOH.

Capillary Electrophoresis-Laser induced fluorescence detector (CE-LIF) is performed as follows. Samples containing about 0.4 mg of EW-XOS are substituted with 5 nmol of maltose as an internal standard. The samples are dried using centrifugal vacuum evaporator (Speedvac). 5 mg of APTS labeling dye (Beckman Coulter) is dissolved in 48 uL of 15% acetic acid (Beckman Coulter). The dried samples are mixed with 2 uL of the labeling dye solution and 2 μl of 1 M Sodium Cyanoborohydride (THF, Sigma-Aldrich). The samples are incubated overnight in the dark to allow the labeling reaction to be completed. After overnight incubation, the labeled samples are diluted 100 times with Millipore water before analysis by CE-LIF. CE-LIF is performed using ProteomeLab PA800 Protein Characterization System (Beckman Coulter), controlled by 32 Karat Software. The capillary column used is polyvinyl alcohol coated capillary (N—CHO capillary, Beckman Coulter), with 50 um ID, 50.2 cm length, 40 cm to detector window. 25 mM sodium acetate buffer pH 4.75 containing 0.4% polyethyleneoxide (Carbohydrate separation buffer. Beckman Coulter) is used as running buffer. The sample (about 3.5 nL) is injected to the capillary by a pressure of 0.5 psi for 3 seconds. The separation is done for 20 minutes at 30 kV separating voltage, with reversed polarity. The labeled XOS are detected using LIF detector at 488 nm excitation and 520 nm emission wavelengths.

This assay can be used to test the activity of enzymes such as, but not limited to, GH67 and GH115 enzymes. Thus, for example, this assay can be used to test the activity of an enzyme such as, but not limited to, an enzyme designated with an activity of “6” in column 2 of Tables 1, 2, 3, or 4.

### Beta-Xylosidase Activity

Xylosidase activity can be assessed using known assays, e.g., by measuring the release of xylose by the action of a xylosidase on xylobiose. An illustrative assay for measuring β-xylosidase activity is as follows. This assay measures the release of p-nitrophenol by the action of β-xylosidase on p-nitrophenyl 1-D-xylopyranoside (PNPX). One β-xylosidase unit of activity is the amount of enzyme that liberates 1 micromole of p-nitrophenol in one minute.

PNPX from Extrasynthese is used as the assay substrate. 16.5 mg of PNPX is dissolved in 5 mL of distilled water and 5 mL 0.1 M sodium acetate buffer pH 5.0 to obtain a 2 mM stock solution. A stop reagent (0.25 M sodium carbonate solution) used to terminate the enzymatic reaction.

0.10 mL of 2 mM PNPX stock solution is mixed with 0.01 mL of the enzyme sample and incubated at 50° C. for 20 minutes. After exactly 30 minutes of incubation, 0.1 mL of 0.25 M sodium carbonate solution is added and then the absorbance at 405 nm (A₄₀₅) is measured in microtiter plates as A_{S} (enzyme sample). A₄₅₀ is also determined for a substrate blank (A_{SB}).

Activity is calculated as follows:

$$Activity ( IU \text{/} ml ) = \frac{Δ {A}_{405} * DF * 21 * 1.33}{13.700 * RT}$$

where ΔA₄₀₅=A_{S}−A_{SB}, DF is the enzyme dilution factor, 21 is the dilution of 10 ul enzyme solution in 210 ul reaction volume, 1.33 is the conversion factor of microtiter plates to cuvettes, 13.700 is the extinction coefficient 13700 M⁻¹ cm⁻¹ of p-nitrophenol released corrected for mol/L to umol/mL, and RT is the reaction time in minutes.

This assay can be used to test the activity of enzymes such as, but not limited to, GH3, GH30, GH39. GH43. GH52, and GH54 enzymes.

An alternative illustrative assay can be used that measures the release of xylose by the action of β-xylosidase on xylobiose. Xylobiose is purchased from Megazyme (Bray Ireland. Cat. #P-WAXYI). 25 mg is dissolved in 5 mL sodium acetate buffer pH 5.0. 5.0 mg/mL substrate solution is mixed with 0.02 mL of the enzyme sample at 50° C., and pH 5.0 for 24 hours. The reaction is stopped by heating the samples for 10 minutes at 100° C. The release of xylose and arabinoxylan oligosaccharides is analyzed by High Performance Anion Exchange Chromatography. A substrate solution blank is also prepared. HPAEC is performed using a Dionex HPLC system equipped with a Dionex CarboPac PA-1 (2 mm ID×250 mm) column in combination with a CarboPac PA guard column (1 mm ID×25 mm) and a Dionex EDet1 PAD-detector (Dionex Co., Sunnyvale). A flow rate of 0.25 mL/min is used with the following gradient of sodium acetate in 0.1 M NaOH: 0-15 min, 0-150 mM. Each elution is followed by a washing step of 5 min using 1 M sodium acetate in 0.1 M NaOH and an equilibration step of 15 min using 0.1 M NaOH.

This assay can be used to test the activity of enzymes such as, but not limited to, GH3, GH30, GH39, GH43, GH52, and GH54 enzymes. Thus, for example, this assay can be used to test the activity of an enzyme such as, but not limited to, an enzyme designated with an activity of “7” in column 2 of Tables 1, 2, 3, or 4.

### Beta-Galactosidase Activity

Beta-galactosidase activity can be assayed using known assays. The following provides an illustrative assay. This assay measures the action of β-galactosidase on 5-Bromo-4-chloro-3-indolyl β-D-galactoside (X-Gal) to yield galactose and 5-bromo-4-chloro-3-hydroxyindole. The compound 5-bromo-4-chloro-3-hydroxyindole is oxidized into 5,5′-dibromo-4,4′-dichloro-indigo, which is an insoluble blue product. X-Gal from Fermentas (St. Leon Rot, Germany) is used as the assay substrate. 1.0 mg of X-Gal is dissolved in 10 mL 0.05 M sodium acetate buffer, pH 5. 0.10 mL of 0.1 mg/mL X-Gal stock solution is mixed with 0.01 mL of the enzyme sample and incubated at 37° C. for 3 hours. After 3 hours of incubation, the absorbance at 590 nm (A₅₉₀) is measured in microtiter plates as A_{S} (enzyme sample). A substrate blank is also prepared and A₅₉₀ is measured (A_{SB}).

Activity is calculated as follows

Activity (IU/ml)=ΔA₅₉₀*DF

where ΔA₅₉₀=A_{S} (enzyme sample)−A_{SB} (substrate blank) and DF is the enzyme dilution factor.

This assay can be used to test the activity of enzymes such as, but not limited to, GH2 and GH42 enzymes.

An illustrative alternative assay is as follows. This assay measures the release of p-nitrophenol by the action of β-galactosidase p-nitrophenyl-P-D-galactopyranoside (PNPGa). One β-galactosidase unit of activity is the amount of enzyme that liberates 1 micromole of p-nitrophenol in one minute. PNPGa (Fluka) is used as the assay substrate. 2.7 mg of PNPGa is dissolved in 10 mL of McIlvain buffer to obtain 1.5 mM stock solution. McIlvain buffer (pH 4.0) is prepared by dissolving 21.01 g of citric acid monohydrate in water to a final volume of 1 L. In a separate container, 53.62 g of Na₂HPO₄*7H₂O is dissolved in water to a volume of 1 L. 614.5 ml of the first solution is mixed with 385.5 mL of the second solution. A stop reagent (0.25 M sodium carbonate) is used to terminate the enzymatic reaction. 0.25 mL of 1.5 mM PNPGa stock solution is mixed with 0.05 mL of the enzyme sample and 0.2 mL buffer and incubated at 37° C. for 10 minutes. After 10 minutes of incubation, 0.5 mL of 1 M Na₂CO₃ solution is added and then the absorbance at 410 nm (A₄₁₀) is measured in microtiter plates as A_{S} (enzyme sample). A substrate blank is also prepared and A₄₁₀ measured A_{SB} (substrate blank sample).

Activity is calculated as follows:

$$Activity ( IU \text{/} ml ) = \frac{Δ {A}_{405} * DF * 21 * 1.33}{13.700 * RT}$$

where ΔA₄₁₀=A_{S} (enzyme sample)−A_{SB} (substrate blank), DF is the enzyme dilution factor, 20 is the dilution of 50 ul enzyme solution in 1000 ul reaction volume, 1.33 is the conversion factor of microtiter plates to cuvettes, 13.700 is the extinction coefficient 13700 M⁻¹ cm⁻¹ of p-nitrophenol released corrected for mol/L to umol/ml, and RT is the reaction time in minutes.

This assay can be used to test the activity of enzymes such as GH2 and GH42. Thus, for example, this assay can be used to test the activity of an enzyme such as, but not limited to, an enzyme designated with an activity of “8” in column 2 of Tables 1, 2, 3, or 4.

### Arabinofuranosidase/Arabinase Activity

Arabinofuranosidase/arabinase activity can be measured using known assays. The following provides an illustrative assay. This assay measures the release of arabinose by the action of the arabinofuranosidase on linear and branched arabinan. Linear and branched arabinan is purchased from British Sugar. The enzyme sample (40-55 μg total protein) is incubated with 5 mg/mL of linear or branched arabinan in 50 mM sodium acetate buffer pH 5.0 at 40° C. for 24 hours. The reaction is stopped by heating the samples at 100° C. for 10 minutes. The samples are centrifuged for 5 minutes at 10,000×g. Degradation of the arabinan is followed by HPAEC analysis. A substrate blank is also prepared. HPAEC is performed using a Dionex HPLC system equipped with a Dionex CarboPac PA-1 (2 mm ID×250 mm) column in combination with a CarboPac PA guard column (1 mm ID×25 mm) and a Dionex EDet1 PAD-detector (Dionex Co., Sunnyvale). A flow rate of 0.3 mL/min is used with the following gradient of sodium acetate in 0.1 M NaOH: 0-40 min, 0-400 mM. Each elution is followed by a washing step of 5 min 1,000 mM sodium acetate in 0.1 M NaOH and an equilibration step of 15 min 0.1 M NaOH.

This assay can be used to test the activity of enzymes such as, but not limited to, GH3, GH43, GH51, GH54, GH62, and GH93 enzymes. Thus, for example, this assay can be used to test the activity of an enzyme such as, but not limited to, an enzyme designated with an activity of “9” in column 2 of Tables 1, 2, 3, or 4.

### Chitin Binding Protein Activity

Chitin binding can be determined using known assays. The following is an illustrative assay. 30 ml fermentation broth is overnight mixed with 5 g chitin in a 50 mL tube at 4° C. A plastic column (6.8×150 mm) is then filled with the mixture and it is washed with water overnight at 4° C. The method is repeated with the unbound material and fresh chitin. The unbound material is analyzed by SDS-gel electrophoresis. The bound proteins, including the matrix, are heated for 10 minutes at 95° C. in sample buffer and separated by SDS-gel electrophoresis. Specific bands from this gel are analyzed by MS/MS.

This assay can be used to test the activity of a protein such as, but not limited to, a protein designated with an activity of “10” in column 2 of Tables 1, 2, 3, or 4.

### Lichenan (Beta (1,3)-Beta(1,4)-Linked Glucan) Binding Protein Activity

Lichenan (which is a beta(1,3)-beta(1,4)-linked glucan) binding can be determined using known assays. The following is an illustrative assay. 30 ml fermentation broth is overnight mixed with 5 g lichenan in a 50 mL tube at 4° C. A plastic column (6.8×150 mm) is then filled with the mixture and it is washed with water overnight at 4° C. The method is repeated with the unbound material and fresh lichenan. The unbound material is analyzed by SDS-gel electrophoresis. The bound proteins, including the matrix, are heated for 10 minutes at 95° C. in sample buffer and separated by SDS-gel electrophoresis. Specific bands from this gel are analyzed by MS/MS.

This assay can be used to test the activity of a protein such as, but not limited to, a protein designated with an activity of “11” in column 2 of Tables 1, 2, 3, or 4.

### Endo-Xylanase Activity

Endo-xylanase activity can be determined using known assays. The following is an illustrative assay. This assay measures endo-xylanase activity towards AZO-wheat arabinoxylan. This substrate is insoluble in buffered solutions, but rapidly hydrates to form gel particles that are readily and rapidly hydrolyzed by specific endo-xylanases releasing soluble dye-labeled fragments. AZO-wheat arabinoxylan (AZO-WAX) from Megazyme (Bray, Ireland, Cat. #I-AWAXP) is used as the assay substrate. 1 g of AZO-WAX is suspended in 3 mL ethanol and adjusted to 100 mL with 0.2 M sodium acetate, pH 5.0. 96% Ethanol is used to terminate the enzymatic reaction. 0.2 mL of 10 mg/ml AZO-WAX stock solution is preheated at 40° C. for 10 minutes. This preheated stock solution is mixed with 0.2 mL of the enzyme sample (preheat at 40° C. for 10 min) and incubated at 40° C. for 10 minutes. After 10 minutes of incubation, 1.0 mL of 96% ethanol is added and then the absorbance at 590 nm (A₅₉₀) is measured as A_{S} (enzyme sample). A substrate blank is also prepared and A₅₉₀ is measured as A_{SB} (substrate blank).

Activity is calculated as follows: endo-xylanase activity is determined by reference to a standard curve, produced from an endo-xylanase with known activity towards AZO-WAX.

Activity (IU/ml)=ΔA₅₉₀/SC*DF

where ΔA₅₉₀=A_{S} (enzyme sample)−A_{SB} (substrate blank), SC is the slope of the standard curve and DF is the enzyme dilution factor.

This assay can be used to test the activity of enzymes such as, but not limited to, GH5, GH8, GH10, and GH11. Thus, for example, this assay can be used to test the activity of an enzyme such as, but not limited to, an enzyme designated with an activity of “12” in column 2 of Tables 1, 2, 3, or 4.

### Xylanase Activity

Xylanase activity can be measured using known assays. An illustrative assay follows. This assay measures the release of xylose and xylo-oligosaccharides by the action of xylanases on wheat arabinoxylan oligosaccharides (WAX). Wheat arabinoxylan is purchased from Megazyme (Bray Ireland, Cat. #P-WAXYI). 5.0 mg/mL of substrate is mixed with 0.05 mg (total protein) of the enzyme sample at 37 CC for 1 hour and 24 hours. The reaction is stopped by heating the samples for 10 minutes at 100° C. The release of xylose and arabinoxylan oligosaccharides are analyzed by High Performance Anion Exchange Chromatography. A substrate blank is also prepared. HPAEC analysis is performed using a Dionex HPLC system equipped with a Dionex CarboPac PA-1 (2 mm ID×250 mm) column in combination with a CarboPac PA guard column (1 mm ID×25 mm) and a Dionex EDet1 PAD-detector (Dionex Co., Sunnyvale). A flow rate of 0.3 mL/min is used with the following gradient of sodium acetate in 0.1 M NaOH: 0-50 min. 0-500 mM. Each elution is followed by a washing step of 5 min 1,000 mM sodium acetate in 0.1 M NaOH and an equilibration step of 15 min 0.1 M NaOH.

This assay can be used to test the activity of enzymes such as, but not limited to, GH5. GH8, GH10, and GH11. Thus, for example, this assay can be used to test the activity of an enzyme such as, but not limited to, an enzyme designated with an activity of “13” in column 2 of Tables 1, 2, 3, or 4.

### Xylan Binding Protein Activity

Xylan binding can be determined using known assays. The following is an illustrative assay to determine the ability of a protein to bind xylan. 30 ml fermentation broth is overnight mixed with 5 g xylan in a 50 mL tube at 4° C. A plastic column (6.8×150 mm) is then filled with the mixture and it is washed with water overnight at 4° C. The method is repeated with the unbound material and fresh xylan. The unbound material is analyzed by SDS-gel electrophoresis. The bound proteins, including the matrix, are heated for 10 minutes at 95° C. in sample buffer and separated by SDS-gel electrophoresis. Specific bands from this gel are analyzed by MS/MS.

This assay can be used to test the activity of a protein such as, but not limited to, a protein designated with an activity of “14” in column 2 of Tables 1, 2, 3, or 4.

### Polygalacturonase Activity

Polygalacturonase activity can be measured using known assays. The following is an illustrative assay for measuring polygalacturonase activity. This assay measures the amount of reducing sugars released from polygalacturonic acid (PGA) by the action of a polygalacturonase. One unit of activity is defined as 1 umole of reducing sugars liberated per minute under the specified reaction conditions. Polygalacturonic acid (PGA) is purchased from Sigma (St. Louis, USA). A working reagent containing PAHBAH is prepared (10 g of p-hydroxy benzoic acid hydrazide (PAHBAH) is suspended in 60 mL water. 10 mL of concentrated HCL is added and the volume adjusted to 200 ml. Reagent B is 24.0 g of trisodium citrated dissolved in 500 ml of water. 2.2 g of calcium chloride and 40 mg of NaOH are added and the volume adjusted to 2 L. with water. Working reagent: 10 ml Reagent A added to 90 ml of Reagent B. 50 uL of PGA (10.0 mg/mL in 0.2 M sodium acetate buffer pH 5.0) is mixed with 30 uL 0.2 M sodium acetate buffer pH 5.0 and 20 uL of the enzyme sample and incubated at 40° C. for 75 minutes. To 25 uL of this reaction mixture, 125 uL of working solution is added. The samples are heated for 5 minutes at 99° C. After cooling down, the samples are analyzed by measuring the absorbance at 410 nm (A₄₁₀) as A_{S} (enzyme sample). A substrate blank is also prepared and A₄₁₀ measured as (A_{SB} (substrate blank sample).

Activity is calculated as follows:

Activity (IU/ml)=ΔA410/SC*DF

where ΔA410=A_{S} (enzyme sample)−A_{SB} (substrate blank), SC is the slope of the standard curve and DF is the enzyme dilution factor.

This assay can be used to test the activity of enzymes such as, but not limited to, GH28. Thus, for example, this assay can be used to test the activity of an enzyme such as, but not limited to, an enzyme designated with an activity of “15” in column 2 of Tables 1, 2, 3, or 4.

### Beta-Glucosidase Activity

Beta-glucosidase activity can be measured using known assays. The following is an illustrative assay for measuring beta-glucosidase activity. This assay measures the release of p-nitrophenol by the action of β-glucosidase on p-nitrophenyl β-D-glucopyranoside (PNPG). One β-glucosidase unit of activity is the amount of enzyme that liberates 1 micromole of p-nitrophenol in one minute. PNPG (Sigma, St. Louis. USA) is used as the assay substrate. 20 mg of PNPG is dissolved in 5 mL of 0.2 M sodium acetate buffer, pH 5.0. 0.25 M Tris-HCl, pH 8.8 is used to terminate the enzymatic reaction. 0.025 mL of PNPG stock solution is mixed with 1 uL of the enzyme sample, 0.075 mL buffer and 0.099 mL water and incubated at 37° C. for 4 minutes. Every minute during 4 minutes a 0.04 mL sample is taken and added to 0.06 mL stop reagent. The absorbance at 410 nm (A₄₁₀) is measured in microtiter plates as A_{S} (enzyme sample). A substrate blank is also prepared and A₄₁₀ measured as A_{SB} (substrate blank sample)

Activity is calculated as follows. The A₄₁₀ values are plotted against time in minutes (X-axis). The slope of the graph is calculated (dA). Enzyme activity is calculated by using the following formula:

$$Specific activity = \frac{\partial A * {V}_{a} * d}{ɛ * l * [ protein ] * {V}_{p}}$$

Where dA=slope in A/min; Va=reaction volume in 1 (0.0002 l); d=dilution factor of assay mix after adding stop reagent (2.5); ε=extinction coefficient (0.0137 μM⁻¹ cm⁻¹); 1=length of cell (0.3 cm); [protein]=protein stock concentration in mg/ml; and Vp=volume of protein stock added to assay (0.001 ml).

This assay can be used to test the activity of enzymes such as, but not limited to, GH1, GH3, GH9, and GH30 enzymes. Thus, for example, this assay can be used to test the activity of an enzyme such as, but not limited to, an enzyme designated with an activity of “16” in column 2 of Tables 1, 2, 3, or 4.

### Beta-1,3-Glucanase Activity

Beta-glucanase activity can be measured using known assays. The following is an illustrative assay for measuring beta-glucanase activity. This assay uses beta-1,3-glucan as the substrate and a reducing sugars assay (PAHBAH) as the detection method. A working reagent containing PAHBAH is prepared (10 g of p-hydroxy benzoic acid hydrazide (PAHBAH) is suspended in 60 mL water. 10 mL of concentrated HCL is added and the volume adjusted to 200 ml. Reagent B is 24.0 g of trisodium citrated dissolved in 500 ml of water. 2.2 g of calcium chloride and 40 mg of NaOH are added and the volume adjusted to 2 L. with water. Working reagent: 10 ml Reagent A added to 90 ml of Reagent B. The assay is performed in a microtiter plate format. 50 uL of β-glucan substrate (1% (w/v) Barley 1-glucan, laminarin, lichenan or curdlan in water), 30 ul of 0.2 M HAc/NaOH pH 5, and 20 ul β-1,3-glucanase sample are used. These reagents are incubated at 37° C. for 2 hours. After incubation, 25 ul of each well are mixed with 125 uL working reagent. The solutions are heated at 95° C. for 5 minutes. After cooling down, the samples are analyzed by measuring the absorbance at 410 nm (A₄₁₀) as A_{S} (enzyme sample). A standard curve is determined and from that the enzyme activities are determined. A substrate blank is also prepared and A₄₁₀ measured for A_{SB} (substrate blank sample).

Activity is calculated as follows: β-1,3-glucanase activity is determined by reference to a standard curve of the cellulase standard solution.

Activity (IU/ml)=ΔA₄₁₀/SC*DF

where ΔA₄₁₀=A_{S} (enzyme sample)−A_{SB} (substrate blank), SC is the slope of the standard curve and DF is the enzyme dilution factor.

This assay can be used to test the activity of enzymes such as the GH5, GH12, GH16, GH17, GH55, GH64 and GH81 enzymes. Thus, for example, this assay can be used to test the activity of an enzyme such as, but not limited to, an enzyme designated with an activity of “17” in column 2 of Tables 1, 2, 3, or 4.

### Alpha-1,6-Mannanase Activity

Alpha-1,6-mannanase activity can be measured using known assays. The following is an illustrative assay. Activity is assed using an alpha-1,6-linked mannobiose as the substrate and a D-mannose detection kit (Megazyme International) as the detection method, using a four enzyme coupled assay, using ATP and NADP+. Reactions are conducted at 37° C. in 100 mM MOPS (pH 7.0), containing 0.1 mM ZnS04, 1 mg mL-1 BSA, and 20 uL of [symbol] 6-Mannanase sample. Mannose liberated by alpha-1,6-Mannanase is phosphorylated to mannose-6-phosphate by hexokinase (HK). Mannose-6-phosphate is subsequently converted to fructose-6-phosphate by phosphomannose isomerase (PMI), which is then isomerized to glucose-6-phosphate by phosphoglucose isomerase (PGI). Finally, glucose-6-phosphate is oxidized to gluconate-6-phosphate by glucose-6-phosphate dehydrogenase (G6P-DH). The concurrent reduction of the NADP+ cofactor to NADPH is monitored at 340 nm using an extinction coefficient of 6223 (M⁻¹-cm⁻¹). The enzymes are individually obtained from Sigma.

Activity is calculated as follows. The A₃₄₀ values are plotted against time in minutes (X-axis). The slope of the graph is calculated (dA). Enzyme activity is calculated by using the following formula:

$$Specific activity = \frac{\partial A * {V}_{a} * d}{ɛ * l * [ protein ] * {V}_{p}}$$

Where dA=slope in A/min; Va=reaction volume in l; d=dilution factor of assay mix; ε=extinction coefficient for NAD(P)H of 0.006223 μM⁻¹ cm⁻¹; l=length of cell in cm; [protein]=protein stock concentration in mg/ml; and Vp=volume of protein stock added to assay in ml.

This assay can be used to test the activity of enzymes such as, but not limited to, GH38, GH76, and GH92 enzymes. Thus, for example, this assay can be used to test the activity of an enzyme such as, but not limited to, an enzyme designated with an activity of “18” in column 2 of Tables 1, 2, 3, or 4.

### Rhamnogalacturonyl Hydrolase Activity

Rhamnogalacturonyl hydrolase activity can be measured using known assays. An illustrative assay follows. Activity is demonstrated using rhamnogalacturonan as a substrate and a reducing sugars assay (PAHBAH) as the detection method. A working reagent containing PAHBAH is prepared (10 g of p-hydroxy benzoic acid hydrazide (PAHBAH) is suspended in 60 mL water. 10 mL of concentrated HCL is added and the volume adjusted to 200 ml. Reagent B is 24.0 g of trisodium citrated dissolved in 500 ml of water. 2.2 g of calcium chloride and 40 mg of NaOH are added and the volume adjusted to 2 L. with water. Working reagent: 10 ml Reagent A added to 90 ml of Reagent B. The assay is conducted in a microtiter plate format. Each well contains 50 uL of rhamnogalacturonan substrate (1%(w/v) in water), 30 uL of 0.2 M HAc/NaOH pH 5, and 20 uL of rhamnogalacturonyl hydrolase sample. These are incubated at 37° C. for 2 hours. After incubation, 25 uL of each well are mixed with 125 uL working reagent. These solutions are heated at 95° C. for 5 minutes. After cooling, the samples are analyzed by measuring the absorbance at 410 nm (A₄₁₀) as A_{S} (enzyme sample). A standard curve is determined and from that the enzyme activities are determined. A substrate blank is also prepared and A₄₁₀ measured for A_{SB} (substrate blank sample).

Activity is calculated as follows: β-1,3-glucanase activity is determined by reference to a standard curve of the cellulase standard solution.

Activity (IU/ml)=ΔA₄₁₀/SC*DF

where ΔA₄₁₀=A_{S} (enzyme sample)−A_{SB} (substrate blank), SC is the slope of the standard curve and DF is the enzyme dilution factor.

This assay can be used to test the activity of enzymes such as, but not limited to, GH28 and GH 105 enzymes. Thus, for example, this assay can be used to test the activity of an enzyme such as, but not limited to, an enzyme designated with an activity of “19” in column 2 of Tables 1, 2, 3, or 4.

### Alpha-Amylase Activity

The activity of Alpha-amylase can be evaluated using known assay. The following ins an illustrative assay. In this assay, activity is demonstrated by using amylose as a substrate and a reducing sugars assay (PAHBAH) as the detection method. A working reagent containing PAHBAH is prepared (10 g of p-hydroxy benzoic acid hydrazide (PAHBAH) is suspended in 60 mL water. 10 mL of concentrated HCL is added and the volume adjusted to 200 ml. Reagent B is 24.0 g of trisodium citrated dissolved in 500 ml of water. 2.2 g of calcium chloride and 40 mg of NaOH are added and the volume adjusted to 2 L. with water. Working reagent: 10 ml Reagent A added to 90 ml of Reagent B. The assay is conducted in a microtiter plate format. Each well contains 50 ul of amylose substrate (0.15% (w/v) in water), 30 ul of 0.2 M HAc/NaOH pH 5, and 20 ul α-amylase sample. The reaction mixture is incubated at 37° C. for 15 minutes. After incubation, 25 ul from each well are mixed with 125 ul working reagent. The solutions are heated at 95° C. for 5 minutes. After cooling, the samples are analyzed by measuring the absorbance at 410 nm (A₄₁₀), A_{S} (enzyme sample). A substrate blank is also prepared and absorbance A₄₁₀ measure, A_{SB} (substrate blank sample.

Alpha-amylase activity is calculated as follows, determined by reference to a standard curve of a cellulase standard solution:

Activity (IU/ml)=ΔA410/SC*DF

where ΔA₄₁₀=A_{S} (enzyme sample)−A_{SB} (substrate blank), SC is the slope of the standard curve and DF is the enzyme dilution factor.

This assay can be used to test the activity of enzymes such as, but not limited to, GH13 and GH57 enzymes. Thus, for example, this assay can be used to test the activity of an enzyme such as, but not limited to, an enzyme designated with an activity of “20” in column 2 of Tables 1, 2, 3, or 4.

### Alpha-Glucosidase Activity

Alpha-glucosidase activity can be determined using known assays. An illustrative assay is as follows. This assay measures the release of p-nitrophenol by the action of α-glucosidase on p-nitrophenyl alpha-D-glucopyranoside. One α-glucosidase unit of activity is the amount of enzyme that liberates 1 micromole of p-nitrophenol in one minute. p-nitrophenyl alpha-D-glucopyranoside (3 mM) (Sigma, #N1377) is used as the assay substrate. 4.52 mg of p-nitrophenyl a-D-glucopyranoside is dissolved in 5 mL of sodium acetate (0.2 M, pH 5.0). Stop reagent (0.25 M Tris-HCl, pH 8.8) is used to terminate the enzymatic reaction. 0.025 mL of p-nitrophenyl a-D-glucopyranoside stock solution is mixed with 1 uL of the enzyme sample, 0.075 mL buffer and 0.099 mL water and incubated at 37° C. for 4 minutes. Every minute during the 4 minutes incubation a 0.04 mL sample is taken and added to 0.06 mL stop reagent. The absorbance at 410 nm (A₄₁₀) is measured in microtiter plates as A_{S} (enzyme sample). A substrate blank is also prepared and the absorbance (A₄₁₀) is measured in microtiter plates as A_{SB} (substrate blank sample).

Activity is calculated as follows. The A₄₁₀ values are plotted against time in minutes (X-axis). The slope of the graph is calculated (dA). Enzyme activity is calculated by using the following formula:

$$Specific activity = \frac{\partial A * {V}_{a} * d}{ɛ * l * [ protein ] * {V}_{p}}$$

Where dA=slope in A/min; Va=reaction volume in l; d=dilution factor of assay mix after adding stop reagent (2.5); ε=extinction coefficient (0.0137 μM⁻¹ cm⁻¹); 1=length of cell (0.3 cm); [protein]=protein stock concentration in mg/ml; and Vp=volume of protein stock added to assay (0.001 ml).

This assay can be used to test the activity of enzymes such as, but not limited to, GH4. GH13. GH31 and GH63 enzymes. Thus, for example, this assay can be used to test the activity of an enzyme such as, but not limited to, an enzyme designated with an activity of “21” in column 2 of Tables 1, 2, 3, or 4.

### Glucoamylase Activity

Glucoamylase activity can be evaluated using known assays. An illustrative assay is as follows. This assay measures the release of p-nitrophenol by the action of glucoamylase on p-nitrophenyl-beta-maltoside (PNPM). One glucoamylase unit of activity is the amount of enzyme that liberates 1 micromole of p-nitrophenol in one minute at 37° C. and pH 5.0. PNPM (Sigma-Aldrich, cat. #N1884) is used as the assay substrate. 18.54 mg of PNPM is dissolved in 5 mL of distilled water and 5 mL 0.1 M acetate buffer, pH 5.0 to obtain a 4 mM stock solution. A stop reagent, 0.1 M sodium tetraborate is used to terminate the enzymatic reaction. For the enzyme sample, 0.04 mL of 4 mM PNPM stock solution is mixed with 0.01 mL of the enzyme sample and incubated at 37° C. for 360 minutes. After 360 minutes of incubation, 0.12 mL of 0.1 M sodium tetraborate solution is added and the absorbance at 405 nm (A₄₀₅) is then measured in microtiter plates as A_{S}. A substrate blank is also prepared and the absorbance A₄₀₅ is measured in microtiter plates as A_{SB}.

Activity is calculated as follows:

$$Activity ( IU \text{/} ml ) = \frac{Δ {A}_{405} * DF * 21 * 1.33}{13.700 * 360}$$

where ΔA₄₀₅=A_{S}−A_{SB}, DF is the enzyme dilution factor, 21 is the dilution of 10 ul enzyme solution in 210 ul reaction volume, 1.33 is the conversion factor of microtiter plates to cuvettes, 13.700 is the extinction coefficient 13700 M⁻¹ cm⁻¹ of p-nitrophenol released corrected for mol/L to umol/mL, and 360 minutes is the reaction time.

This assay can be used to test the activity of enzymes such as, but not limited to, GH15 enzymes. Thus, for example, this assay can be used to test the activity of an enzyme such as, but not limited to, an enzyme designated with an activity of “22” in column 2 of Tables 1, 2, 3, or 4.

### Glucanase Activity

Glucanase activity can be measure using assays well known in the art. The following is an illustrative assay. Activity is demonstrated by using a glucan (e.g. dextran, glycogen, pullulan, amylose, amylopectin, cellulose, curdlan, laminarin, chrysolaminarin, lentinan, lichenin, pleuran, zymosan, etc.) as the substrate and a reducing sugars assay (PAHBAH) as the detection method. A working reagent containing PAHBAH is prepared (10 g of p-hydroxy benzoic acid hydrazide (PAHBAH) is suspended in 60 mL water. 10 mL of concentrated HCL is added and the volume adjusted to 200 ml. Reagent B is 24.0 g of trisodium citrated dissolved in 500 ml of water. 2.2 g of calcium chloride and 40 mg of NaOH are added and the volume adjusted to 2 L. with water. Working reagent: 10 ml Reagent A added to 90 ml of Reagent B. The assay is conducted in a microtiter plate format. Each well contains 50 ul of glucan substrate (1% (w/v) glucan in water), 30 ul of 0.2 M HAc/NaOH pH 5, 20 ul glucanase sample. These are incubated at 37° C. for 2 hours. After incubation, 25 ul of each well are mixed with 125 ul working reagent. The solutions are heated at 95° C. for 5 minutes. After cooling, the samples are analyzed by measuring the absorbance at 410 nm (A₄₁₀) as A_{S} (enzyme sample). A substrate blank is also prepared and absorbance (A₄₁₀) measured as A_{SB} (substrate blank sample.) A standard curve is determined and from that the enzyme activities are determined.

Activity is calculated as follows: glucanase activity is determined by reference to a standard curve of a standard solution.

Activity (IU/ml)=ΔA₄₁₀/SC*DF

where ΔA₄₁₀=A_{S} (enzyme sample)−A_{SB} (substrate blank), SC is the slope of the standard curve and DF is the enzyme dilution factor.

This assay can be used to test the activity of enzymes such as, but not limited to, GH5, GH6, GH7, GH8, GH9, GH12, GH13, GH14, GH15, GH16, GH17, GH30, GH44, GH48, GH49, GH51, GH55, GH57, GH64, GH71, GH74, and GH81 enzymes. Thus, for example, this assay can be used to test the activity of an enzyme such as, but not limited to, an enzyme designated with an activity of “23” in column 2 of Tables 1, 2, 3, or 4.

### Acetyl Esterase Activity

Acetyl esterase activity can be measured using known assays. The following is an illustrative assay. This assay measures the release of p-nitrophenol by the action of acetyl esterase on p-nitrophenyl acetate (PNPAc). One acetyl esterase unit of activity is the amount of enzyme that liberates 1 micromole of p-nitrophenol in one minute at 37° C. and pH 5. PNPAc (Fluka, cat. #46021) is used as the assay substrate. 3.6 mg of PNPAc is dissolved in 10 mL of 0.05 M sodium acetate buffer, pH 5.0 to obtain a 2 mM stock solution. A stop reagent (0.25 M Tris-HCl, pH 8.8) is used to terminate the enzymatic reaction. 0.10 mL of 2 mM PNPAc stock solution is mixed with 0.01 mL of the enzyme sample and incubated at 37° C. for 10 minutes. After 10 minutes of incubation, 0.1 mL of 0.25 M Tris-HCl solution is added and the absorbance at 405 nm (A₄₀₅) is measured in microtiter plates as A_{S} (enzyme sample). A substrate blank is also prepared and the absorbance A₄₀₅ is measured in microtiter plates as A_{SB} (substrate blank).

Activity is calculated as follows:

$$Activity ( IU \text{/} ml ) = \frac{Δ {A}_{405} * DF * 21 * 1.33}{13.700 * RT}$$

where ΔA₄₀₅=A_{S}−A_{SB}, DF is the enzyme dilution factor, 21 is the dilution of 10 ul enzyme solution in 210 ul reaction volume, 1.33 is the conversion factor of microtiter plates to cuvettes, 13.700 is the extinction coefficient 13700 M⁻¹ cm⁻¹ of p-nitrophenol released corrected for mol/L to □mol/mL, and RT is the reaction time in minutes.

This assay can be used to test the activity of enzymes such as, but not limited to, CE1, CE2, CE3, CE4, CE5, CE6, CE7, CE12, CE13 and CE16 enzymes. Thus, for example, this assay can be used to test the activity of an enzyme such as, but not limited to, an enzyme designated with an activity of “24” in column 2 of Tables 1, 2, 3, or 4.

### Acetyl Xylan Esterase Activity

Acetyl xylan esterase activity can be measured using assays known in the art. An illustrative assay follows. This assay measures acetyl xylan esterase activity towards arabinoxylan oligosaccharides from Eucalyptus wood by measuring the release of acetate by the action of the acetyl xylan esterases on the arabinoxylan oligosaccharides. Acetylated, 4-O-MeGlcA substituted xylo-oligosaccharides with 2-10 xylose residues from Eucalyptus globulus wood (EW-XOS), Eucalyptus globulus wood AIS and Eucalyptus globulus xylan polymer are obtained using known methods. 5 mL of substrate solution, containing 1 mg EW-XOS in water is mixed with 0.5% (w/w) enzyme/substrate ratio and incubated at 40° C. and pH 7 for 24 hours. The reaction is stopped by heating the samples for 10 minutes at 100° C. The release of acetic acid and formation of new (arabino)xylan oligosaccharides are analyzed by Matrix-Assisted Laser Desorption/Ionization Time-Of-Flight Mass Spectrometry and Capillary Electrophoresis. A substrate blank is also prepared.

Matrix-Assisted Laser Desorption/Ionization Time-Of-Flight Mass Spectrometry (“MALDI-TOF MS”) is performed as follows. An Ultraflex workstation (Bruker Daltronics GmbH, Germany) is used with a nitrogen laser at 337 nm. The mass spectrometer is calibrated with a mixture of malto-dextrins (mass range 365-2309). The samples are mixed with a matrix solution (1 each). The matrix solution is prepared by dissolving 10 mg of 2,5-dihydroxybenzoic acid in a 1 mL mixture of water in order to prepare a saturated solution. After thorough mixing, the solution is centrifuged to remove undissolved material. 1 ul of the prepared sample and 1 ul of matrix solution is put on a gold plate and dried with warm air.

Capillary Electrophoresis-Laser induced fluorescence detector (“CE-LIF”) is performed as follows. Samples containing about 0.4 mg of EW-XOS are substituted with 5 nmol of maltose as an internal standard. The samples are dried using a centrifugal vacuum evaporator. 5 mg of APTS labeling dye (Beckman Coulter) is dissolved in 48 ul of 15% acetic acid (Beckman Coulter). The dried samples are mixed with 2 μl of the labeling dye solution and 2 ul of 1 M Sodium Cyanoborohydride (THF, Sigma-Aldrich). The samples are incubated overnight in the dark to allow the labeling reaction to be completed. After overnight incubation, the labeled samples are diluted 100 times with Millipore water before analysis by CE-LIF. CE-LIF is performed using ProteomeLab PA800 Protein Characterization System (Beckman Coulter), controlled by 32 Karat Software. The capillary column used is polyvinyl alcohol coated capillary (N—CHO capillary, Beckman Coulter), having 50 μm ID, 50.2 cm length and 40 cm to detector window. 25 mM sodium acetate buffer pH 4.75 containing 0.4% polyethyleneoxide (Carbohydrate separation buffer, Beckman Coulter) is used as running buffer. The sample (ca. 3.5 nL) is injected to the capillary by a pressure of 0.5 psi for 3 seconds. The separation is done for 20 minutes at 30 kV separating voltage, with reversed polarity. During analysis, the samples are stored at 10° C. The labeled EW-XOS are detected using LIF detector at 488 nm excitation and 520 nm emission wavelengths.

This assay can be used to test the activity of enzymes such as, but not limited to, CE 1, CE2, CE3, CE4, CE5, CE6, CE7, CE 12, and CE 16 enzymes. Thus, for example, this assay can be used to test the activity of an enzyme such as, but not limited to, an enzyme designated with an activity of “25” in column 2 of Tables 1, 2, 3, or 4.

### Ferulic Acid Esterase Activity

Ferulic acid esterase activity can be measured using known assays. The following is an illustrative assay. This assay measures the release of p-nitrophenol by the action of ferulic acid esterase on p-nitrophenylbutyrate (PNBu). One ferulic acid esterase unit of activity is the amount of enzyme that liberates 1 micromole of p-nitrophenol in one minute at 37° C., pH 7.2. PNPBu (Sigma, cat. #N9876-5G) is used as the assay substrate. 10 ul of PNPBu is mixed with 25 ml of 0.01 M phosphate buffer, pH 7.2 to obtain a 2 mM stock solution. A stop reagent (0.25 M Tris-HCl, pH 8.5) is used to terminate the enzymatic reaction. For the enzyme sample, 0.10 mL of 2 mM PNBu stock solution is mixed with 0.01 mL of the enzyme sample and incubated at 37° C. for 10 minutes. After 10 minutes of incubation, 0.10 mL of 0.25 M Tris HCl pH 8.8 is added and the absorbance at 405 nm (A₄₀₅) is then measured in microtiter plates as A_{S}. A substrate blank is also prepared and the absorbance A₄₀₅ is measured in microtiter plates as Ass.

Activity is calculated as follows:

$$Activity ( IU \text{/} ml ) = \frac{Δ {A}_{405} * DF * 21 * 1.33}{13.700 * 10}$$

where ΔA₄₀₅=A_{S}−A_{SB}, DF is the enzyme dilution factor, 21 is the dilution of 10 ul enzyme solution in 210 ul reaction volume, 1.33 is the conversion factor of microtiter plates to cuvettes, 13.700 is the extinction coefficient 13700 M⁻¹ cm⁻¹ of p-nitrophenol released corrected from mol/L to umol/mL, and 10 is the reaction time in minutes.

This assay can be used to test the activity of enzymes such as, but not limited to, an enzyme designated with an activity of “26” in column 2 of Tables 1, 2, 3, or 4.

The following assay is an alternative assay to measure ferulic acid esterase activity. In this assay, ferulic acid esterase activity is measured using wheat bran (WB) oligosaccharides and measuring the release of ferulic acid. Wheat bran oligosaccharides are prepared by degradation of wheat bran (Nedalco, The Netherlands) by endo-xylanase III from A. niger. 50 mg of WB is dissolved in 10 ml of 0.05 M acetate buffer pH 5.0. 1.0 ml of WB stock solution is mixed with 0.0075 mg of the enzyme and incubated at 35° C. for 24 hours. The reaction is stopped by heating the samples for 10 minutes at 100° C. The residual material is removed by centrifugation (15 minutes at 14000 rpm), and the supernatant is used as the substrate in the assay detailed below.

For the enzyme sample, 1.0 ml of wheat bran oligosaccharides stock solution is mixed with 0.005 mg of the enzyme sample and incubated at 35° C. for 24 hours. The reaction is stopped by heating the samples for 10 minutes at 100° C. The release of ferulic acid is analyzed by measuring the absorbance at 335 nm. A substrate blank is also prepared and used as a control.

This assay can be used to test the activity of enzymes such as, but not limited to, an enzyme designated with an activity of “27” in column 2 of Tables 1, 2, 3, or 4.

### Glucuronyl Esterase Activity

Glucuronyl esterase activity can be measured using known assays. The following is an illustrative assay. This assay measures the release of 4-O-methyl-glucuronic acid by the action of the glucuronyl esterases on methyl-4-O-methyl-glucuronic acid. 200 uL of methyl-4-O-methyl-glucuronic acid stock solution (0.5 mg/mL) is mixed with 10 uL of the enzyme sample and incubated at 30° C. for 4 hours. The reaction is stopped by heating the samples for 15 minutes at 99° C. The release of glucose is analyzed by UPLC-MS. A substrate blank is also prepared for a control.

This assay can be used to test the activity of enzymes such as, but not limited to, an enzyme designated with an activity of “28” in column 2 of Tables 1, 2, 3, or 4.

### Endo-Glucanase Activity

Endo-glucanase activity can be measure using known assays. The following is an illustrative assay. Activity is demonstrated by using a glucan (e.g. dextran, glycogen, pullulan, amylose, amylopectin, cellulose, curdlan, laminarin, chrysolaminarin, lentinan, lichenin, pleuran, zymosan, etc.) as substrate and a reducing sugars assay (PAHBAH) as a detection method. A working reagent containing PAHBAH is prepared (10 g of p-hydroxy benzoic acid hydrazide (PAHBAH) is suspended in 60 mL water. 10 mL of concentrated HCL is added and the volume adjusted to 200 ml. Reagent B is 24.0 g of trisodium citrated dissolved in 500 ml of water. 2.2 g of calcium chloride and 40 mg of NaOH are added and the volume adjusted to 2 L. with water. Working reagent: 10 ml Reagent A added to 90 ml of Reagent B. The assay is conducted in a microtiter plate format. Each well contains 50 ul of glucan substrate (1% (w/v) glucan in water), 30 ul of 0.2 M sodium acetate, pH 5, and 20 ul endo-glucanase sample. These are incubated at 37° C. for 2 hours. After incubation 25 ul from each well are mixed with 125 ul working reagent. These solutions are heated at 95° C. for 5 minutes. After cooling, the samples are analyzed by measuring the absorbance at 410 nm (A₄₁₀) as A_{S} (enzyme sample). A standard curve is determined and from that the enzyme activities are determined. A substrate blank is also prepared and the absorbance (A410) measured as A_{SB} (substrate blank sample).

Activity is calculated as follows: endo-glucanase activity is determined by reference to a standard curve of the cellulase standard solution.

Activity (IU/ml)=ΔA₄₁₀/SC*DF

where ΔA₄₁₀=A_{S}−A_{SB}.

This assay can be used to test the activity of enzymes such as, but not limited to, an enzyme designated with an activity of “29” in column 2 of Tables 1, 2, 3, or 4.

### Alpha-Glucanase Activity

a-glucanase activity can be measured using known assays. An illustrative assay is as follows. Activity is demonstrated by using an alpha-glucan (e.g. dextran, glycogen, pullulan, amylopectin, amylose, etc.) as the substrate and a reducing sugars assay (PAHBAH) as a detection method. A working reagent containing PAHBAH is prepared (10 g of p-hydroxy benzoic acid hydrazide (PAHBAH) is suspended in 60 mL water. 10 mL of concentrated HCL is added and the volume adjusted to 200 ml. Reagent B is 24.0 g of trisodium citrated dissolved in 500 ml of water. 2.2 g of calcium chloride and 40 mg of NaOH are added and the volume adjusted to 2 L. with water. Working reagent: 10 ml Reagent A added to 90 ml of Reagent B. The assay is conducted in a microtiter plate format. Each well contains 50 ul of alpha-glucan substrate (1% (w/v) alpha-glucan in water), 50 ul of 0.2 M sodium acetate pH 5, and 20 ul alpha-glucanase sample. These are incubated at 37° C. for 2 hours. After incubation, 25 ul from each well are mixed with 125 ul working reagent. These solutions are heated at 95° C. for 5 minutes. After cooling, the samples are analyzed by measuring the absorbance at 410 nm (A₄₁₀) as A_{S} (enzyme sample). A substrate blank is also prepared and absorbance (A₄₁₀) measured as A_{SB} (substrate blank sample.) A standard curve is determined and from that the enzyme activities are determined.

Activity is calculated as follows: a-glucanase activity is determined by reference to a standard curve of cellulase standard solution.

Activity (IU/ml)=ΔA₄₁₀/SC*DF

where ΔA₄₁₀=A_{S} (enzyme sample)−A_{SB} (substrate blank), SC is the slope of the standard curve and DF is the enzyme dilution factor.

This assay can be used to test the activity of enzymes such as, but not limited to, an enzyme designated with an activity of “30” in column 2 of Tables 1, 2, 3, or 4.

### Beta-Glucanase Activity

Beta-glucanase activity can be measured using known assays. An illustrative assay is as follows. Activity is demonstrated by using [beta-glucan as a substrate and a reducing sugars assay (PAHBAH) as a detection method. A working reagent containing PAHBAH is prepared (10 g of p-hydroxy benzoic acid hydrazide (PAHBAH) is suspended in 60 mL water. 10 mL of concentrated HCL is added and the volume adjusted to 200 ml. Reagent B is 24.0 g of trisodium citrated dissolved in 500 ml of water. 2.2 g of calcium chloride and 40 mg of NaOH are added and the volume adjusted to 2 L. with water. Working reagent: 10 ml Reagent A added to 90 ml of Reagent B. The assay is conducted in a microtiter plate format. Each well contains 50 ul of beta-glucan substrate (1%(w/v) Bailey beta-glucan in water), 30 ul of 0.2 M HAc NaOH pH 5, and 20 ul □-glucanase sample. These are incubated at 37° C. for 2 hours. After incubation, 25 ul from each well are mixed with 125 ul working reagent. The solutions are heated at 95° C. for 5 minutes. After cooling, the samples are analyzed by measuring the absorbance at 410 nm (A₄₁₀) as A_{S} (enzyme sample). A standard curve is determined and from that the enzyme activities are determined. A substrate blank is also prepared and absorbance (A₄₁₀) measured as A_{SB} (substrate blank sample.)

Activity is calculated as follows: beta-glucanase activity is determined by reference to a standard curve of cellulase standard solution.

Activity (IU/ml)=ΔA₄₁₀/SC*DF

where ΔA₄₁₀=A_{S} (enzyme sample)−A_{SB} (substrate blank), SC is the slope of the standard curve and DF is the enzyme dilution factor.

This assay can be used to test the activity of enzymes such as, but not limited to, an enzyme designated with an activity of “31” in column 2 of Tables 1, 2, 3, or 4.

### Alpha-Galactosidase Activity

Alpha-galactosidase activity can be measured using known assays. An illustrative assay using 4-Nitrophenyl-alpha-D-galactopyranoside is as follows. The substrate (100 ul of 2 mM 4-Nitrophenyl-alpha-D-galactopyranoside in 50 mM NaAc pH5.0) is mixed with 10 ul of sample in wells of a microtiter plate. 100 ul of 0.25 M NaCO₃ is added to stop the solution after 10 minutes incubation at 37° C. Samples are then measured in a plate reader at E410 nm.

To quantify activity, timed samples are taken and the specific activity is calculated as follows: E410 nm is plotted as the Y-axis and time in minutes as the X-axis. The slope of the graph (Y/X) is calculated. Enzyme activity is calculated by using the following formula:

$$Specific activity = \frac{\partial A * {V}_{r} * d * {D}_{e}}{ɛ * l * [ protein ] * {V}_{p}}$$

where dA=slope in A/min; Vr=reaction volume in l; De=enzyme dilution before addition to reaction mix; d=dilution factor of assay mix after adding stop reagent; ε=extinction coefficient (0.0158 uM⁻¹ cm⁻¹); 1=length of cell (1.0 cm in case of cuvettes); [protein]=protein stock concentration in mg/ml; vp=volume of protein solution added to assay in ml.

This assay can be used to test the activity of enzymes such as, but not limited to, an enzyme designated with an activity of “32” in column 2 of Tables 1, 2, 3, or 4.

### Beta-Mannosidase Activity

Beta-mannosidase activity can be measured using assays known in the art. An illustrative assay using 2 mM 4-Nitrophenyl-beta-D-mannopyranoside as a substrate is as follows. The substrate (100 ul of 2 mM 4-Nitrophenyl-beta-D-annopyranoside in 50 mM NaAc pH5.0) is mixed with 10 ul of sample in wells of a microtiter plate. 100 ul of 0.25 M NaCO₇ is added to stop the solution after 10 minutes incubation at 37° C. Samples are then measured in a plate reader at E410 nm.

To quantify activity, timed samples are taken and the specific activity is calculated as follows: E410 nm is plotted as the Y-axis and time in minutes as the X-axis. The slope of the graph (Y/X) is calculated. Enzyme activity is calculated by using the following formula:

$$Specific activity = \frac{\partial A * {V}_{r} * d * {D}_{e}}{ɛ * l * [ protein ] * {V}_{p}}$$

where dA=slope in A/min; Vr=reaction volume in l; De=enzyme dilution before addition to reaction mix; d=dilution factor of assay mix after adding stop reagent; ε=extinction coefficient (0.0158 uM⁻¹ cm⁻¹); l=length of cell (1.0 cm in case of cuvettes); [protein]=protein stock concentration in mg/ml; vp=volume of protein solution added to assay in ml.

This assay can be used to test the activity of enzymes such as, but not limited to, an enzyme designated with an activity of “33” in column 2 of Tables 1, 2, 3, or 4.

### Rhamnogalacturonan Acetyl Esterase Activity

Rhamnogalacturonan acetyl esterase activity can be measured using known assays. An illustrative assay is as follows. This assay measures the release of acetic acid by the action of the rhamnogalacturonan acetyl esterase on sugar beet pectin. Sugar beet pectin is from CP Kelco (Atlanta, USA). The acetic acid assay kit from Megazyme (Bray, Ireland). The rhamnogalacturonan acetyl esterase sample is incubated with sugar beet pectin at 50° C. in 10 mM phosphate buffer pH 7.0 during 16 hours of incubation. The E/S ratio is 0.5% (5 ug enzyme/mg substrate). The total volume of the reaction is 110 uL. The released acetic acid is analyzed with the acetic acid assay kit according to instructions of the supplier. The enzyme with known rhamnogalacturonan acetyl esterase activity Rgael (CL1 1462) is used as a reference.

This assay can be used to test the activity of enzymes such as, but not limited to, an enzyme designated with an activity of “34” in column 2 of Tables 1, 2, 3, or 4.

### α-Fucosidase Activity

Alpha-fucosidase activity can be measured using assay known in the art. An illustrative assaying follows. This assay uses p-nitrophenyl a-L-fucoside as substrate. The enzyme sample (30 to 50 μl containing 5˜10 μg protein) is added to 0.25 ml of 2 mM substrate dissolved in 50 mM sodium citrate buffer (pH 4.5). After incubation at 37° C., 1.75 ml of 0.2 M sodium borate buffer (pH 9.8) is added to terminate the reaction and the release of p-nitrophenol is determined by measuring absorbance at 400 nm (A₄₀₀). One unit of enzyme activity is the amount of enzyme that releases 1 μmol of p-nitrophenol per min. The specific activity is expressed as unit/mg of protein.

This assay can be used to test the activity of enzymes such as, but not limited to, an enzyme designated with an activity of “43” in column 2 of Tables 1, 2, 3, or 4.

### α-Xylosidase Activity

The activity of an α-xylosidase can be measured using assays known in the art. The following are two illustrative assays. In one assay, α-xylosidase activity is assessed with a colorimetric assay using p-nitrophenyl-α-D-xyloside as substrate. The enzyme sample (30 to 50 μl containing 5˜10 μg protein) is added to 0.25 ml of 2 mM substrate dissolved in 50 mM sodium citrate buffer (pH 4.5). After incubation for an appropriate time at 37° C., 1.75 ml of 0.2 m sodium borate buffer (pH 9.8) is added to terminate the reaction and the release of p-nitrophenol is determined by measuring absorbance and 400 nm (A₄₀₀). A substrate blank is prepared as a control. One unit of the enzyme activity is defined as the amount of enzyme which releases 1 μmol of p-nitrophenol per min. The specific activity is expressed as unit/mg of protein.

Alternatively, the activity of α-xylosidase can be measured using tamarind xyloglucan (XG). Because XG contains β-linked Gal and β-linked Glc in addition to α-linked Xyl, four enzymes are included in the experiment: xyloglucanase, β-glucosidase, and β-galactosidase, in addition to α-xylosidase. A high-throughput 4-component design of experiment (DoE) experiment is performed setting the lower limit of all four enzymes to 5%. All enzymes are added at a range of loading between 5% and 85% of 15 ug total enzyme loading/reaction. A stock solution of tamarind XG is 2.5 mg/ml in 50 mM citrate buffer pH 5.0. The reaction plates are incubated at 50° C. for 48 hrs at 10 rpm. At the end of the reaction, the glucose and xylose released from the hydrolysate are measured by HPLC. Complete digestion of tamarind XG should be achieved releasing Glc and Xyl. The DoE model should predict the efficiency of the α-xylosidase, and its contribution towards the complete deconstruction of tamarind XG (see. e.g., Scott-Craig et al. 2011. J. Biol. Chem. 286:42848-54, 2011, which is herein incorporated by reference).

This assay can be used to test the activity of enzymes such as, but not limited to, an enzyme designated with an activity of “44” in column 2 of Tables 1, 2, 3, or 4.

### Laccase Activity

Laccase activity can be measured using assays well known in the art. The following is an illustrative assay. In this assay, laccase activity is determined by oxidation of 2,2′-azino-bis(3-ethylbenzthiazoline-6-sulfonic acid) (ABTS) substrate. The reaction mixture contains 5 mM ABTS in 0.1 M sodium acetate buffer (pH 5.0) and a suitable amount of enzyme. Oxidation of ABTS is followed by monitoring absorbance increase at 420 nm (A₄₂₀). The enzyme activity is expressed in units defined as the amount of enzyme oxidizing 1 μmol of ABTS min⁻¹ (ε420=36.000 M⁻¹ cm⁻¹).

This assay can be used to test the activity of enzymes such as, but not limited to, an enzyme designated with an activity of “45” in column 2 of Tables 1, 2, 3, or 4.

### Protease Activity

Protease activity can be assayed using well known methods. For example, activity of some proteases can be determined by measurement of degradation of protease substrates in solution, such as bovine serum albumin (BSA), as described by van den Hombergh et al. (Curr Genet 28:299-308, 1995, which is herein incorporated by reference). As the protease enzymes digest the protein in suspension, the mixture becomes more transparent and the absorbance changes in the reaction mixture can be followed spectophotometrically.

In an alternative illustrative assay, activity of some proteases can be determined by measurement of degradation of AZCL-casein in solution as described by the manufacturer (Megazyme, Ireland). As the protease enzyme digests the AZCL-casein in suspension, the mixture becomes blue and the absorbance changes in the reaction mixture can be followed spectophotometrically.

Further, assays for peptidase activity are well known in the art. One of skill will be able to choose the appropriate assay for the desired enzyme activity. For example, U.S. Pat. No. 6,184,020 teaches aminopeptidase assays; and U.S. Pat. No. 6,518,054 teaches metallo endopeptidase assays.

A protease assay can be used to test the activity of enzymes such as, but not limited to, an enzyme designated with an activity of “35” in column 2 of Tables 1, 2, 3, or 4.

### Oxidase Activity

Oxidase activity can be measured using known assays. An oxidase catalyzes an oxidation-reduction reaction involving molecule oxygen as the electron acceptors. In these reactions, oxygen is reduced to water or hydrogen peroxide. An example of an assay to measure oxidase activity is thus an assay that measures oxygen consumption, using a Clark electrode (Clark, L. C. Jnr. Ann. NY Acad. Sci. 102, 29-45, 1962) at a specific temperature in an air-saturated sample containing its substrate (e.g. glucose and galactose, for glucose oxidase and galactose oxidase, respectively). The reaction can be initiated by injection of a catalytic amount of oxidase in the oxygen electrode chamber. Kinetic parameters can be determined by measuring initial rates at different substrate concentrations.

An oxidase assay can be used to test the activity of enzymes such as, but not limited to, an enzyme designated with an activity of “36” in column 2 of Tables 1, 2, 3, or 4.

### Peroxidase Activity

Peroxidase activity can be measured using known assays. An illustrative assay is based on the oxidation of 2,2′-azino-di(3-ethylbenzthiazoline-6-sulphonate) (ABTS) from Sigma-Aldrich (e.g., Gallati, V. H. J. Clin. Chem. Clin. Biochem. 17, 1, 1979, which is herein incorporated by reference). The absorbance increase of the oxidized form of ABTS, measured at 410 nm, is proportional to the peroxidase activity. The assay may also be used to indirectly measure oxidase activity. The formation of hydrogen peroxide, catalyzed by the oxidase, is coupled to the oxidation of ABTS by the addition of a peroxidase (e.g. horseradish peroxidase).

A peroxidase assay can be used to test the activity of enzymes such as, but not limited to, an enzyme designated with an activity of “37” in column 2 of Tables 1, 2, 3, or 4.

### Reductase Activity

Reductase activity can be assayed using methods well known in the art. An illustrative assay for measuring nitrate reductase activity is described by Garrett & Cove, Mol. Gen. Genet. 149:179-186, 2006, which is herein incorporated by reference.

A reductase assay can be used to test the activity of enzymes such as, but not limited to, an enzyme designated with an activity of “38” in column 2 of Tables 1, 2, 3, or 4.

### Dehydrogenase Activity

Dehydrogenase activity can be determined using well known assays. In an illustrative assay, dehydrogenase activity is assessed by measuring the decrease in absorbance at 340 nm resulting from the oxidation of the NADH or NADPH cofactor when incubated with a substrate. For example, the activity of glycerol 3-phosphate dehydrogenase (GPDH), can be determined by measuring the decrease in absorbance at 340 nm when the enzyme was incubated with dihydroxyacetone phosphate as a substrate (e.g., Arst et al. Mol Gen Genet. 1990 August; 223(1): 134-137, which is herein incorporated by reference).

A dehydrogenase assay can be used to test the activity of enzymes such as, but not limited to, an enzyme designated with an activity of “39” in column 2 of Tables 1, 2, 3, or 4.

### Cutinase Activity

Cutinase activity can be determined using well known assays. An example of such an assay is an esterase assay performed using spectrophotometry (e.g., Davies et al., Physiol. Mol. Plant Pathol. 57:63-75, 2000, which is herein incorporated by reference) with p-nitrophenyl butyrate as a substrate. Cutinase activity can also be measured using ³H-labelled apple cutin as a substrate by an adaptation of the method of Koller et al., Physiol. Plant Pathol. 20:47-60, 1982, which is herein incorporated by reference.

A cutinase assay can be used to test the activity of enzymes such as, but not limited to, an enzyme designated with an activity of “40” in column 2 of Tables 1, 2, 3, or 4.

### Pectin Acetyl Esterase or Rhamnogalacturonan Acetyl Esterase Activity

Pectin acetyl esterase or rhamnogalacturonan acetyl esterase activity can be measured using known assays. In an illustrative assay, the release of acetic acid by the action of the pectin acetyl esterase or rhamnogalacturonan acetyl esterase activity is measured. Sugar beet pectin (CP, Kelco) is used as a substrate. The acetic acid assay kit is obtained from Megazyme. The pectin acetyl esterase or rhamnogalacturonan acetyl esterase enzyme sample is incubated at 50° C. in 10 mM phosphate buffer pH 7.0 during 16 hours of incubation. The E/S ratio is 0.5% (5 □g enzyme/mg substrate). The total volume of the reaction is 110 □L. The released ac [symbol] analyzed with the acetic acid assay kit according to instructions of the supplier. Enzyme with known pectin acetyl esterase or rhamnogalacturonan acetyl esterase activity is used as a reference.

This assay can be used to test the activity of enzymes such as, but not limited to, an enzyme designated with an activity of “41” in column 2 of Tables 1, 2, 3, or 4.

Measurement of Activity for Increasing Protein Productivity and/or Saccharification Efficiency

The ability of a polypeptide of the invention to increase protein productivity and/or saccharification efficiency can be measured using known assays. The following is an illustrative assay for assessing the effects of a protein on increased protein productivity and/or saccharification efficiency using Myceliophthora thermophila host cells. Myceliophthora thermophila strain(s) transformed with nucleic acid constructs that express a protein of interest, e.g., a polypeptide of Tables 1, 2, 3, or 4 are generated using standard methods known in the art. The resulting strains are grown in liquid culture using standard methods, e.g., as described in Example 1. The cells are separated from the culture medium by centrifugation. The culture medium containing proteins secreted by the fungal strain are assayed for the total amount of protein produced/secreted. The samples are first de-salted using Bio-Rad Econo-Pac 10DG Columns (Bio-Rad, Cat. No. 732-2010) as per the manufacturer's suggestions. The total protein present in the samples is assayed using a BCA protein assay kit (Thermo-Scientific, Pierce Protein Biology Products, Product No. 23225), as per the manufacturer's suggestions and the amount of protein production is compared to control strains that have not been transformed with a nucleic acid construct encoding the protein of interest. Transformants that produce increased amounts of secreted proteins compared to the controls exhibit increased protein productivity. An “increase” in protein productivity is typically at least 10%, or at least 20% or greater, in comparison to a control cell.

The produced/secreted polypeptides (as obtained from the process described above) are directly tested for increased saccharification performance. For this purpose, the samples are tested either before or after the de-salting step (as described in the previous section). The reactions employ 10-20% Avicel substrate (CAS Number 9004-34-6, Sigma-Aldrich, Product No. 11365-1KG), 0.5-1% produced enzyme with respect to substrate (wt/wt), at pH5-6, 55° C., for 24-72 h while shaking. The reactions are heat quenched at 85° C. at 850 RPM for 15 min, and filtered through a 0.45 μm filter. The samples are then assayed for the production of the final product glucose using a standard GOPOD assay kit (for example, Megazyme, Catalog No. K-GLUC), as per the manufacturer's directions. Any other cellulose-containing material can be employed in this assay (for example, pre-treated biomass), and the enzyme addition can be volume-based (wt of substrate to volume of enzyme). M. thermophila transformants that express that produce increased amounts of saccharification activity are identified by this process. An “increase” in saccharification is typically at least 10%, or at least 20% or greater, in comparison to a control cell. Cells that produce increased amounts of proteins and provide for increased amounts of hydrolysis activity are identified using the combination of the two assays.

These assays can be used to test the activity of polypeptides such as, but not limited to, a polypeptide designated with an activity of “42” in column 2 of Tables 1, 2, 3, or 4.

### IV. Biomass Degradation and Protein Productivity Polynucleotides and Expression Systems

The present invention provides polynucleotide sequences that encode biomass degradation polypeptides. Exemplary cDNA sequences encoding biomass degradation polypeptides of the invention are each identified by a sequence identifier in Column 3 of Table 1, Table 2, Table 3, and Table 4 with reference to the appended sequence listing. The invention also provides polynucleotide sequences that encode protein productivity polypeptides. Exemplary cDNA sequences encoding protein productivity polypeptides of the invention are each identified by a sequence identifier in Column 3 of Table 1, Table 2, Table 3, and Table 4 with reference to the appended sequence listing. These sequences encode the respective polypeptides shown in the tables, which are each identified by a sequence identifier with reference to the appended sequence listing. Those having ordinary skill in the art will readily appreciate that due to the degeneracy of the genetic code, a multitude of nucleotide sequences encoding a polypeptide of Table 1, Table 2, Table 3, and Table 4 exist. For example, the codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence. The invention contemplates and provides each and every possible variation of nucleic acid sequence encoding a polypeptide of the invention that could be made by selecting combinations based on possible codon choices.

A DNA sequence may also be designed for high codon usage bias codons (codons that are used at higher frequency in the protein coding regions than other codons that code for the same amino acid). The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. In particular, a DNA sequence can be optimized for expression in a particular host organism. See GCG CodonPreference, Genetics Computer Group Wisconsin Package; Codon W, John Peden, University of Nottingham; McInerney, J. O, 1998, Bioinformatics 14:372-73; Stenico et al., 1994, Nucleic Acids Res. 222437-46; Wright, F., 1990, Gene 87:23-29; Wada et al., 1992, Nucleic Acids Res. 20:2111-2118; Nakamura et al., 2000, Nucl. Acids Res. 28:292, all of which are incorporated herein by reference.

### Expression Vectors

The present invention makes use of recombinant constructs comprising a sequence encoding a polypeptide of Tables 1, 2, 3, or 4. In a particular aspect, the present invention provides an expression vector encoding a polypeptide of Tables 1, 2, 3, or 4, e.g., a glycohydrolase, wherein the polynucleotide encoding the polynucleotide is operably linked to a heterologous promoter. Expression vectors of the present invention may be used to transform an appropriate host cell to permit the host to express the polypeptide. Methods for recombinant expression of proteins in fungi and other organisms are well known in the art, and any number of expression vectors are available or can be constructed using routine methods. See, e.g., Tkacz and Lange, 2004, ADVANCES IN FUNGAL BIOTECHNOLOGY FOR INDUSTRY, AGRICULTURE, AND MEDICINE, KLUWER ACADEMIC/PLENUM PUBLISHERS. New York; Zhu et al., 2009, Construction of two Gateway vectors for gene expression in fungi Plasmid 6:128-33; Kavanagh, K. 2005, FUNGI: BIOLOGY AND APPLICATIONS Wiley, all of which are incorporated herein by reference.

Nucleic acid constructs of the present invention comprise a vector, such as, a plasmid, a cosmid, a phage, a virus, a bacterial artificial chromosome (BAC), a yeast artificial chromosome (YAC), and the like, into which a nucleic acid sequence encoding a polypeptide of Tables 1, 2, 3, or 4 has been inserted. The nucleic acids can be incorporated into any one of a variety of expression vectors suitable for expressing a polypeptide. Suitable vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others. Any vector that transduces genetic material into a cell, and, if replication is desired, which is replicable and viable in the relevant host can be used.

In an aspect of this embodiment, the construct further comprises regulatory sequences, including, for example, a promoter, operably linked to the protein encoding sequence. Large numbers of suitable vectors and promoters are known to those of skill in the art. The construct may optionally include nucleotide sequences to facilitate integration into a host genome and/or results in amplification of construct copy number in vivo.

### Promoter/Gene Constructs

As discussed above, to obtain high levels of expression in a particular host it is often useful to express a polypeptide of the invention under control of a heterologous promoter. Typically a promoter sequence may be operably linked to the 5′ region of the biomass degradation protein coding sequence. It will be recognized that in making such a construct it is not necessary to define the bounds of a minimal promoter. Instead, the DNA sequence 5′ to the lignocellulose degradation gene start codon can be replaced with DNA sequence that is 5′ to the start codon of a given heterologous gene (e.g., a C1 sequence from another gene, or a promoter from another organism). This 5′ “heterologous” sequence thus includes, in addition to the promoter elements per se, a transcription start signal and the sequence of the 5′ untranslated portion of the transcribed chimeric mRNA. Thus, the promoter-gene construct and resulting mRNA will comprise a sequence encoding a polypeptide of Tables 1, 2, 3, or 4 and a heterologous 5′ sequence upstream to the start codon of the sequence encoding the polypeptide. In some, but not all, cases the heterologous 5′ sequence will immediately abut the start codon of the polynucleotide sequence encoding the polypeptide. In some embodiments, gene constructs may be employed in which a polynucleotide encoding a polypeptide of Tables 1, 2, 3, or 4 is present in multiple copies. Such embodiments may employ the endogenous promoter for the gene encoding the polypeptide or may employ a heterologous promoter.

In one embodiment, a polypeptide of Tables 1, 2, 3, or 4 is expressed as a pre-protein including the naturally occurring signal peptide of the polypeptide. In some embodiments, polypeptide of the invention that is expressed has a sequence of column 4 in Table 1 or Table 3.

In one embodiment, the polypeptide is expressed from the construct as a pre-protein with a heterologous signal peptide.

In some embodiments, a heterologous promoter is operably linked to a polypeptide cDNA nucleic acid sequence of Column 3 of Tables 1, 2, 3, or 4.

Examples of useful promoters for expression of polypeptides of the invention include promoters from fungi. For example, promoter sequences that drive expression of homologous or orthologous genes from other organisms may be used. For example, a fungal promoter from a gene encoding a glyohydrolase, e.g., a cellobiohydrolase, may be used.

Examples of other suitable promoters useful for directing the transcription of the nucleotide constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787, which is incorporated herein by reference), as well as the NA2-tpi promoter (a hybrid of the promoters from the genes for Aspergillus niger neutral alpha-amylase and Aspergillus oryzae triose phosphate isomerase), promoters such as cbh1, cbh2, egl1, egl2, pepA, hfb1, hfb2, xyn1, amy, and glaA (Nunberg et al., Mol. Cell Biol., 4:2306-2315 (1984), Boel et al., EMBO J. 3:1581-1585 ((1984) and EPA 137280, all of which are incorporated herein by reference), and mutant, truncated, and hybrid promoters thereof. In a yeast host, useful promoters can be from the genes for Saccharomyces cerevisiae enolase (ENO-1). Saccharomyces cerevisiae galactokinase (GAL 1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8:423-488. Promoters associated with chitinase production in fungi may be used. See, e.g., Blaiseau and Lafay, 1992, Gene 120243-248 (filamentous fungus Aphanocladium album); Limon et al., 1995, Curr. Genet. 28:478-83 (Trichoderma harzianum), both of which are incorporated herein by reference.

Promoters known to control expression of genes in prokaryotic or eukaryotic cells or their viruses that can be used in some embodiments of the invention include SV40 promoter, E. coli lac or trp promoter, phage lambda P_{L} promoter, tac promoter. T7 promoter, and the like. In bacterial host cells, suitable promoters include the promoters obtained from the E. coli lac operon, Streptomyces coelicolor agarase gene (dagA), Bacillus subtilis levansucranse gene (sacB), Bacillus licheniformis alpha-amylase gene (amyl). Bacillus slearothermophilus maltogenic amylase gene (amyM). Bacillus amyloliquefaciens alpha-amylase gene (amyQ), Bacillus subtilis xylA and xylB genes and prokaryotic β-lactamase gene.

An expression vector can contain other sequences, for example, an expression vector may optionally contain a ribosome binding site for translation initiation, and a transcription terminator. The vector also optionally includes appropriate sequences for amplifying expression, e.g., an enhancer.

In addition, expression vectors that encode a polypeptide of the invention optionally contain one or more selectable marker genes to provide a phenotypic trait for selection of transformed host cells. Suitable marker genes include those coding for antibiotic resistance such as, ampicillin (ampR), kanamycin, chloramphenicol, or tetracycline resistance. Further examples include the antibiotics spectinomycin (e.g., the aada gene); streptomycin, e.g., the streptomycin phosphotransferase (SPT) gene coding for streptomycin resistance; the neomycin phosphotransferase (NPTII) gene encoding kanamycin or geneticin resistance; the hygromycin phosphotransferase (HPT) gene coding for hygromycin resistance. Additional selectable marker genes include dihydrofolate reductase or neomycin resistance for eukaryotic cell culture, and tetracycline or ampicillin resistance in E. coli. Selectable markers for fungi include markers for resistance to HPT, phleomycin, benomyl, and acetamide.

### Synthesis and Manipulation of Polynucleotides

Polynucleotides encoding a polypeptide of Tables 1, 2, 3, or 4 can be prepared using methods that are well known in the art. For example, individual oligonucleotides may be individually synthesized, then joined (e.g., by enzymatic or chemical ligation methods, or polymerase-mediated methods) to form essentially any desired continuous sequence. Chemical synthesis of oligonucleotides can be performed using, for example, the classical phosphoramidite method described by Beaucage, et al., 1981, Tetrahedron Letters, 22:1859-69, or the method described by Matthes, et al., 1984, EMBO J. 3:801-05, both of which are incorporated herein by reference. These methods are typically practiced in automated synthetic methods. In a chemical synthesis method, oligonucleotides are synthesized, e.g., in an automatic DNA synthesizer, purified, annealed, ligated and cloned in appropriate vectors. Further, essentially any nucleic acid can be custom ordered from any of a variety of commercial sources.

General texts that describe molecular biological techniques that are useful herein, including the use of vectors, promoters, protocols sufficient to direct persons of skill through in vitro amplification methods, including the polymerase chain reaction (PCR) and the ligase chain reaction (LCR), and many other relevant methods, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y. 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”), all of which are incorporated herein by reference. Reference is made to Berger. Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564, all of which are incorporated herein by reference. Methods for cloning in vitro amplified nucleic acids are described in Wallace et al., U.S. Pat. No. 5,426,039, which is incorporated herein by reference.

### Expression Hosts

The present invention also provides engineered (recombinant) host cells that are transformed with an expression vector or DNA construct encoding a polypeptide of Tables 1, 2, 3, or 4. As used herein, a genetically modified or recombinant host cell includes the progeny of said host cell that comprises a polynucleotide that encodes a recombinant polypeptide of Tables 1, 2, 3, or 4. In some embodiments, the genetically modified or recombinant host cell is a eukaryotic cell. Suitable eukaryotic host cells include, but are not limited to, fungal cells, algal cells, insect cells, and plant cells. In some cases, host cells may be modified to increase protein expression, secretion or stability, or to confer other desired characteristics. Cells (e.g., fungi) that have been mutated or selected to have low protease activity are particularly useful for expression. For example, Myceliophthora thermophila strains in which the alp1 (alkaline protease) locus has been deleted or disrupted may be used. Many expression hosts can be employed in the invention, including fungal host cell, such as yeast cells and filamentous fungal cells; algal host cells; and prokaryotic cells, including gram positive, gram negative and gram-variable bacterial cells. Examples are listed below.

Suitable fungal host cells include, but are not limited to, Ascomycota, Basidiomycota, Deuteromycota, Zygomycota, Fungi imperfecti. Particularly preferred fungal host cells are yeast cells and filamentous fungal cells. The filamentous fungal host cells of the present invention include all filamentous forms of the subdivision Eumycotina and Oomycota. (see, for example, Hawksworth et al., In Ainsworth and Bisby's Dictionary of The Fungi, 8^{th} edition, 1995, CAB International, University Press, Cambridge. UK, which is incorporated herein by reference). Filamentous fungi are characterized by a vegetative mycelium with a cell wall composed of chitin, cellulose and other complex polysaccharides. The filamentous fungal host cells of the present invention are morphologically distinct from yeast.

In some embodiments the filamentous fungal host cell may be a cell of a species of, but not limited to Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothia, Fusarium, Gibberella, Gliocladium, Humicola, Hypocrea, Myceliophthora, Mucor, Neurosxpora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, Volvariella, or teleomorphs, or anamorphs, and synonyms or taxonomic equivalents thereof.

In some embodiments of the invention, the filamentous fungal host cell is of the Aspergillus species, Ceriporiopsis species, Chrysosporium species. Corynascus species, Fusarium species, Humicola species, Neurospora species, Penicillium species, Tolypocladium species, Tramates species, or Trichoderma species.

In some embodiments of the invention, the filamentous fungal host cell is of the Trichoderma species, e.g., T. longibrachiatum, T. viride (e.g., ATCC 32098 and 32086), Hypocrea jecorina or T. reesei (NRRL 15709, ATTC 13631, 56764, 56765, 56466, 56767 and RL-P37 and derivatives thereof—See Sheir-Neiss et al., 1984, Appl. Microbiol. Biotechnology, 20:46-53, which is incorporated herein by reference), T. koningii, and T. harzianum. In addition, the term “Trichoderma” refers to any fungal strain that was previously classified as Trichoderma or currently classified as Trichoderma.

In some embodiments of the invention, the filamentous fungal host cell is of the Aspergillus species, e.g., A. awanori, A. fumigatus, A. japonicus, A. nidulans, A. niger, A. aculeatus, A. foetidus, A. oryzae, A. sojae, and A. kawachi. (Reference is made to Kelly and Hynes, 1985, EMBO J. 4,475479; NRRL 3112, ATCC 11490, 22342, 44733, and 14331; Yelton et al., 1984, Proc. Natl. Acad. Sci. USA. 81, 1470-1474; Tilburn et al., 1982, Gene 26,205-221; and Johnston et al., 1985, EMBO J. 4, 1307-1311, all of which are incorporated herein by reference).

In some embodiments of the invention, the filamentous fungal host cell is of the Fusarium species, e.g., F. hactridioides, F. cerealis, F. crookwellense, F. culmorum, F. graminearum, F. graminum. F. oxyspxorum, F. roseum, and F. venenalum. In some embodiments of the invention, the filamentous fungal host cell is of the Neurospora species, e.g., N. crassa. Reference is made to Case, M. E. et al., (1979) Proc. Natl. Acad. Sci. USA, 76, 5259-5263; U.S. Pat. No. 4,486,553; and Kinsey, J. A. and J. A. Rambosek (1984) Molecular and Cellular Biology 4, 117-122, all of which are incorporated herein by reference. In some embodiments of the invention, the filamentous fungal host cell is of the Humicola species, e.g., H. insolens, H. grisea, and H. lanuginosa. In some embodiments of the invention, the filamentous fungal host cell is of the Mucor species, e.g., M. miehei and M. circinelloides. In some embodiments of the invention, the filamentous fungal host cell is of the Rhizopus species, e.g., R. oryzae and R. niveus. In some embodiments of the invention, the filamentous fungal host cell is of the Penicillum species, e.g., P. purpurogenum, P. chrysogenum, and P. verruculosum. In some embodiments of the invention, the filamentous fungal host cell is of the Thielavia species, e.g., T. terrestris. In some embodiments of the invention, the filamentous fungal host cell is of the Tolypocladium species, e.g., T. inflatum and T. geodes. In some embodiments of the invention, the filamentous fungal host cell is of the Trametes species, e.g., T. villosa and T. versicolor.

In some embodiments of the invention, the filamentous fungal host cell is of the Chrysosporium species, e.g., C. lucknowense, C. keralinophilum, C. tropicum, C. merdarium, C. inops. C. pannicola, and C. zonatum. In a particular embodiment the host is Myceliophthora thermophila.

In the present invention a yeast host cell may be a cell of a species of, but not limited to Candida, Hansenula, Saccharomyces, Schizosaccharomyces, Pichia, Kluyveromyces, and Yarrowia. In some embodiments of the invention, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, and Yarrowia lipolytica.

In some embodiments on the invention, the host cell is an algal such as, Chlamydomonas (e.g., C. reinhardtii) and Phormidium (P. sp. ATCC29409).

In other embodiments, the host cell is a prokaryotic cell. Suitable prokaryotic cells include gram positive, gram negative and gram-variable bacterial cells. The host cell may be a species of, but not limited to, Agrobacterium, Alicyclobacillus, Anabaena, Anacystic, Acinetobacten, Acidothermus, Arthrobacter, Azobacter Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mevorhizobtum, Methylobacterium, Mrycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synechococcus, Saccharomonopora, Staphylococcus, Serratia, Salmonella, Shigella, Thermoanaerobacterium, Tropheryma, Tularensis, Temecula, Thermosynechoccus, Thermococcus, Ureaplasma, Xanthomnonas, Xylella, Yersinia and Zymomonas.

In some embodiments, the host cell is a species of Agrobacterium, Acinetobacter. Azobacter, Bacillus, Bifidobacterium, Buchnera, Geobacillus, Campylobacter, Clostridium, Corynebacterium, Escherichia, Enterococcus, Erwinia, Flavobacterium, Lactobacillus, Lactococcus, Pantoea, Pseudomonas, Staphylococcus, Salmonella, Streptococcus, Streptomyces, and Zymomonas.

In yet other embodiments, the bacterial host strain is non-pathogenic to humans. In some embodiments the bacterial host strain is an industrial strain. Numerous bacterial industrial strains are known and suitable in the present invention.

In some embodiments of the invention the bacterial host cell is of the Agrobacterium species, e.g., A. radiobacter. A. rhizogenes, and A. rubi. In some embodiments of the invention the bacterial host cell is of the Arthrobacter species, e.g., A. aurescens, A. citreus, A. globformis, A. hydrocarboglutamicus, A. mysorens, A. nicotianae, A. paraffineus, A. prolophonniae, A. roseoparaffinus, A. sulfureus, and A. ureafaciens. In some embodiments of the invention the bacterial host cell is of the Bacillus species, e.g., B. thuringiensis, B. anthracis, B. megaterium, B. subtilis, B. lentus, B. circulans. B. pumilus, B. lautus, B. coagulans. B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. Some preferred embodiments of a Bacillus host cell include B. subtilis. B. licheniformis, B. megaterium, B. stearothermophilus and B. amyloliquefaciens. In some embodiments the bacterial host cell is of the Clostridium species. e.g., C. acetobutylicium, C. tetani E88, C. lituseburense, C. saccharobutylicum, C. perfringens, and C. beijerinckii. In some embodiments the bacterial host cell is of the Cornebacterium species e.g., C. glutamicum and C. acetoacidophilum. In some embodiments the bacterial host cell is of the Escherichia species, e.g., E. coli. In some embodiments the bacterial host cell is of the Erwinia species, e.g., E. uredovora, E. carotovora, E. ananas, E. herbicola, E. punctata, and E. terreus. In some embodiments the bacterial host cell is of the Pantoea species, e.g., P. citrea, and P. agglomerans. In some embodiments the bacterial host cell is of the Pseudomonas species, e.g., P. putida. P. aeruginosa, P. mevalonii, and P. sp. D-01 10. In some embodiments the bacterial host cell is of the Streptococcus species, e.g., S. equisimiles, S. pyogenes, and S. uberis. In some embodiments the bacterial host cell is of the Streptomyces species, e.g., S. ambofaciens, S. achromogenes, S. avermitilis, S. coelicolor, S. aureofaciens, S. aureus, S. fungicidicus, S. griseus, and S. lividans. In some embodiments the bacterial host cell is of the Zymomonas species, e.g., Z. mobilis, and Z. lipolytica.

Strains that may be used in the practice of the invention including both prokaryotic and eukaryotic strains, are readily accessible to the public from a number of culture collections such as American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Host cells may be genetically modified to have characteristics that improve protein secretion, protein stability or other properties desirable for expression and/or secretion of a protein. Genetic modification can be achieved by genetic engineering techniques or using classical microbiological techniques, such as chemical or UV mutagenesis and subsequent selection. A combination of recombinant modification and classical selection techniques may be used to produce the organism of interest. Using recombinant technology, nucleic acid molecules can be introduced, deleted, inhibited or modified, in a manner that results in increased yields of a biomass degradation polypeptide of the invention, e.g., a glycohydrolase set forth in Tables 1, 2, 3, or 4, within the organism or in the culture. For example, knock out of pyr5 function results in a cell with a pyrimidine deficient phenotype.

### Transformation

Introduction of a vector or DNA construct into a host cell can be effected by calcium phosphate transfection, DEAE-Dextran mediated transfection, electroporation, or other common techniques (See Davis et al., 1986, Basic Methods in Molecular Biology, which is incorporated herein by reference). Transformation of Myceliophthora thermophila host cells is known in the art (see, e.g., US 2008/0194005 which is incorporated herein by reference).

### Culture Conditions

The engineered host cells can be cultured in conventional nutrient media modified as appropriate for activating promoters, selecting transformants, or amplifying the lignocellulose degradation enzyme polynucleotide. Culture conditions, such as temperature, pH and the like, are those previously used with the host cell selected for expression, and will be apparent to those skilled in the art. As noted, many references are available for the culture and production of many cells, including cells of bacterial, plant, animal (especially mammalian) and archaebacterial origin. See e.g., Sambrook, Ausubel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelli, et al., (1989) In vitro Cell Dev. Biol. 25:1016-1024, all of which are incorporated herein by reference. For plant cell culture and regeneration. Payne et al. (1992) Plant Cell and Tissue Culture in Liquid Systems John Wiley & Sons. Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); Jones, ed. (1984) Plant Gene Transfer and Expression Protocols, Humana Press, Totowa, N.J. and Plant Molecular Biology (1993) R. R. D. Croy, Ed. Bios Scientific Publishers, Oxford, U.K. ISBN 0 12 198370 6, all of which are incorporated herein by reference. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue (1998) from Sigma-Aldrich, Inc (St Louis, Mo.) (“Sigma-LSRCCC”) and, for example, The Plant Culture Catalogue and supplement (1997) also from Sigma-Aldrich, Inc (St Louis, Mo.) (“Sigma-PCCS”), all of which are incorporated herein by reference.

Culture conditions for fungal cells, e.g., Myceliophthora thermophila host cells are known in the art and can be readily determined by one of skill. See, e.g., US 2008/0194005, US 20030187243, WO 2008/073914 and WO 01/79507, which are incorporated herein by reference.

### V. Production and Recovery of Polypeptides

In one aspect, the invention is directed to a method of making a polypeptide having an amino acid sequence of Tables 1, 2, 3, or 4, the method comprising providing a host cell transformed with a polynucleotide encoding the polypeptide, e.g., a nucleic acid of Tables 1, 2, 3, or 4; culturing the transformed host cell in a culture medium under conditions in which the host cell expresses the encoded polypeptide; and optionally recovering or isolating the expressed polypeptide, or recovering or isolating the culture medium containing the expressed polypeptide. The method further provides optionally lysing the transformed host cells after expressing the polypeptide and optionally recovering or isolating the expressed polypeptide from the cell lysate.

In a further embodiment, the present invention provides a method of over-expressing (i.e., making,) a polypeptide having an amino acid sequence of Tables 1, 2, 3, or 4, e.g., a biomass degradation polypeptide of Tables 1, 2, 3, or 4, comprising: (a) providing a recombinant Myceliophthora thermophila host cell comprising a nucleic acid construct, wherein the nucleic acid construct comprises a polynucleotide sequence that encodes a polypeptide of Tables 1, 2, 3, or 4 and the nucleic acid construct optionally also comprises a polynucleotide sequence encoding a signal peptide at the amino terminus of polypeptide, wherein the polynucleotide sequence encoding the polypeptide and optional signal peptide is operably linked to a heterologous promoter; and (b) culturing the host cell in a culture medium under conditions in which the host cell expresses the encoded polypeptide, wherein the level of expression of the polypeptide from the host cell is greater, preferably at least about 2-fold greater, than that from wildtype Myceliophthora thermophila cultured under the same conditions. The signal peptide employed in this method may be any heterologous signal peptide known in the art or may be a wildtype signal peptide of a sequence set forth in Column 4 of Table 1 or Table 3. In some embodiments, the level of overexpression is at least about 5-fold, 10-fold, 12-fold, 15-fold, 20-fold, 25-fold, 30-fold, or 35-fold greater than expression of the protein from wildtype cells.

Typically, recovery or isolation of the polypeptide, e.g., a biomass degradation polypeptide, is from the host cell culture medium, the host cell or both, using protein recovery techniques that are well known in the art, including those described herein. Cells are typically harvested by centrifugation, disrupted by physical or chemical means, and the resulting crude extract may be retained for further purification. Microbial cells employed in expression of proteins can be disrupted by any convenient method, including freeze-thaw cycling, sonication, mechanical disruption, or use of cell lysing agents, or other methods, which are well known to those skilled in the art.

The resulting polypeptide may be recovered/isolated and optionally purified by any of a number of methods known in the art. For example, a biomass degradation polypeptide of the invention may be isolated from the nutrient medium by conventional procedures including, but not limited to, centrifugation, filtration, extraction, spray-drying, evaporation, chromatography (e.g., ion exchange, affinity, hydrophobic interaction, chromatofocusing, and size exclusion), or precipitation. Protein refolding steps can be used, as desired, in completing the configuration of the mature protein. Finally, high performance liquid chromatography (HPLC) can be employed in the final purification steps. As a further illustration, purification of a glycohydrolase is described in US patent publication US 2007/0238155, incorporated herein by reference. In addition to the references noted supra, a variety of purification methods are well known in the art, including, for example, those set forth in Sandana (1997) Bioseparation of Proteins, Academic Press, Inc.; Bollag et al. (1996) Protein Methods, 2^{nd} Edition, Wiley-Liss, NY; Walker (1996) The Protein Protocols Handbook Humana Press, NJ; Harris and Angal (1990) Protein Purification Applications: A Practical Approach, IRL Press at Oxford, Oxford, England; Harris and Angal Protein Purification Methods: A Practical Approach, IRL Press at Oxford, Oxford, England; Scopes (1993) Protein Purification: Principles and Practice 3^{rd} Edition, Springer Verlag, NY; Janson and Ryden (1998) Protein Purification: Principles, High Resolution Methods and Applications, Second Edition. Wiley-VCH, NY; and Walker (1998) Protein Protocols on CD-ROM, Humana Press, NJ, all of which are incorporated herein by reference.

Immunological methods may also be used to purify a polypeptide of the invention. In one approach, an antibody raised against the enzyme using conventional methods is immobilized on beads, mixed with cell culture media under conditions in which the enzyme is bound, and precipitated. In a related approach immunochromatograpy is used. In some embodiments, purification is achieved using protein tags to isolate recombinantly expressed protein.

### VI. Cells Having Absent or Decreased Expression of a Polypeptide of the Invention

In some embodiments, a host cell is genetically modified to disrupt expression of a polypeptide of Tables 1, 2, 3, or 4. The term “disrupted” as applied to expression of a gene refers to any genetic modification that decreases or eliminates the expression of the gene and/or the functional activity of the corresponding gene product (mRNA and/or protein). In one embodiment the disruption eliminates or substantially reduces expression of the gene product as determined by, for example, immunoassays. “Substantially reduce”, in this context, means the amount of expressed protein is reduced by at least 50%, often at least 75%, sometimes at least 80%, at least 90% or at least 95% compared to expression from the undisrupted gene. In some embodiments, a gene product (e.g., protein) is expressed from the disrupted gene but the protein is mutated (e.g., comprises a deletion, insertion of substitution(s)) that completely or substantially reduce the biological activity of the protein. In some embodiments, a disruption may completely eliminate expression, i.e., the gene produce has no measurable activity. “Substantially reduce”, in this context, means expression or activity of a protein is reduced by at least 50%, often at least 75%, sometimes at least 80%, at least 90% or at least 950% compared to a cell that is not genetically modified to disrupt expression of the gene of interest.

Methods of disrupting expression of a gene are well known, and the particular method used to reduce or abolish the expression of the endogenous gene is not critical to the invention. For example, in some embodiments, a genetically modified host cell with disrupted expression of a gene of interest has a deletion of all or a portion of the protein-encoding sequence of the endogenous gene, a mutation in the endogenous gene such that the gene encodes a polypeptide having no activity or reduced activity (e.g., insertion, deletion, point, or frameshift mutation), reduced expression due to antisense RNA or small interfering RNA that inhibits expression of the endogenous gene, or a modified or deleted regulatory sequence (e.g., promoter) that reduces expression of the endogenous gene, any of which may bring about a disrupted gene. In some embodiments, all of the genes disrupted in the microorganism are disrupted by deletion. Illustrative references describing deletion of all or part of the gene encoding the protein and site-specific mutagenesis to disrupt expression or activity of the gene product include Chaveroche et al., 2000, Nucleic Acids Research, 28:22 e97; Cho et al., 2006, MPMI 19: 1, pp. 7-15; Maruyama and Kitamoto, 2008, Biotechnol Lett 30:1811-1817; Takahashi et al., 2004, Mol Gen Genomics 272: 344-352; and You et al., 2009, Arch Micriobiol 191:615-622. In alternative methods, random mutagenesis using chemical mutagens or insertions mutagenesis can be employed to disrupt gene expression.

Additional methods of inhibiting expression of a polypeptide of Tables 1, 2, 3, or 4 include use of siRNA, antisense, or ribozyme technology to target a nucleic acid sequence that encodes a polypeptide of Tables 1, 2, 3, or 4. Such techniques are well known in the art. Thus, the invention further provides a sequence complementary to the nucleotide sequence of a gene encoding a polypeptide of the invention that is capable of hybridizing to the mRNA produced in the cell to inhibit the amount of protein expressed.

Host cells, e.g., Myceliophthora thermophila cells, manipulated to inhibit expression of a polypeptide of the invention can be screened for decreased gene expression using standard assays to determine the levels of RNA and/or protein expression, which assays include quantitative RT-PCR, immunoassays and/or enzymatic activity assays. Host cells with disrupted expression can be as host cells for the expression of native and/or heterologous polypeptides.

Thus, in a further aspect, the invention additionally provides a recombinant host cell comprising a disruption or deletion of a gene encoding a polypeptide identified in Tables 1, 2, 3, or 4, wherein the disruption or deletion inhibits expression of the polypeptide encoded by the polynucleotide sequence. In some embodiments, the recombinant host cell comprises an antisense RNA or iRNA that is complementary to a polynucleotide sequence identified in Tables 1, 2, 3, or 4.

### VII. Methods of Using Polypeptides of the Invention and Cells Expressing the Polypeptides

As described supra, polypeptides of the present invention and/or host cells expression the polypeptides can be used in processes to degrade cellulosic biomass. For example, a biomass degradation polypeptide such as a glycoside hydrolase of Tables 1, 2, 3, or 4 can be used to catalyze the hydrolysis of a sugar dimer with the release of the corresponding sugar monomer. In some embodiments, polypeptide of the invention participates in the degradation of cellulosic biomass to obtain a carbohydrate not by directly hydrolyzing cellulose or hemicellulose to obtain the carbohydrate, but by generating a degradation product that is more readily hydrolyzed to a carbohydrate by cellulases and accessory proteins. For example, lignin can be broken down using a biomass degradation enzyme of the invention, such as a laccase, to provide an intermediate in which more cellulose or hemicellulose is accessible for degradation by cellulases and glycoside hydrolases. Various other enzymes, e.g., endoglucanases and cellobiohydrolases catalyze the hydrolysis of insoluble cellulose to cellooligosaccharides while beta-glucosidases convert the oligosaccharides to glucose. Similarly, xylanases, together with other enzymes such as alpha-L-arabinofuranosidases, ferulic and acetylxylan esterases and beta-xylosidases, catalyze the hydrolysis of hemicelluloses.

The present invention thus further provides compositions that are useful for the enzymatic conversion of a cellulosic biomass to soluble carbohydrates. For example, one or more biomass degradation polypeptides of the present invention may be combined with one or more other enzymes and/or an agent that participates in biomass degradation. The other enzyme(s) may be a different glycoside hydrolase or an accessory protein such as an esterase, oxidase, or the like; or an ortholog, e.g., from a different organism of an enzyme of the invention.

In some embodiments, a host cell that is genetically modified to overexpress a polypeptide of Tables 1, 2, 3, or 4 can be used to produce increased amount of proteins, e.g., for use in biomass degradation processes.

### Cellulosic Biomass Degradation Mixtures

For example, in some embodiments, a glycoside hydrolase biomass degradation enzyme set forth in Tables 1, 2, 3, or 4 may be combined with other glycoside hydrolases to form a mixture or composition comprising a recombinant biomass degradation polypeptide of the present invention and a Myceliophthora thermophila cellulase or other filamentous fungal cellulase. The mixture or composition may include cellulases selected from CBH, EG and BG cellulases (e.g., cellulases from a Trichoderma sp. (e.g. Trichoderma reesei and the like); an Acidothermus sp. (e.g., Acidothermus cellulolyticus, and the like); an Aspergillus sp. (e.g., Aspergillus nidulans, Aspergillus niger, Aspergillus oryzae, and the like); a Humicola sp. (e.g., Humicola grisea, and the like); a Chrysosporium sp., as well as cellulases derived from any of the host cells described under the section entitled “Expression Hosts”, supra).

The mixture may additionally comprise one or more accessory proteins, e.g., an accessory enzyme such as an esterase to de-esterify hemicellulose, set forth in Tables 1, 2, 3, or 4; and/or accessory proteins from other organisms. The enzymes of the mixture work together resulting in hydrolysis of the hemicellulose and cellulose from a biomass substrate to yield soluble carbohydrates, such as, but not limited to, glucose and xylose (See Brigham et al., 1995, in Handbook on Bioethanol (C. Wyman ed.) pp 119-141, Taylor and Francis, Washington D.C., which is incorporated herein by reference). In some embodiments, mixtures of purified naturally occurring or recombinant enzymes are combined with cellulosic biomass or a product of lignocellulose hydrolysis. Alternatively or in addition, one or more cells producing naturally occurring or recombinant biomass degradation enzymes may be used.

### Other Components of Enzyme Compositions

Biomass degradation enzymes of the present invention may be used in combination with other optional ingredients such as a buffer, a surfactant, and/or a scouring agent. A buffer may be used with an enzyme of the present invention (optionally combined with other cellulose degradation enzymes) to maintain a desired pH within the solution in which the enzyme is employed. The exact concentration of the buffer employed will depend on several factors which the skilled artisan can determine. Suitable buffers are well known in the art. A surfactant may further be used in combination with the enzymes of the present invention. Suitable surfactants include any surfactant compatible with the cellulose degradation enzyme of the invention and optional other enzymes being utilized. Exemplary surfactants include anionic, non-ionic, and ampholytic surfactants.

Production of Soluble Sugars from Cellulosic Biomass

Biomass degradation polypeptides of the present invention, as well as any composition, culture medium, or cell lysate comprising such polypeptides, may be used in the production of monosaccharides, disaccharides, or oligomers of a mono- or di-saccharide from biomass for subsequent use as chemical or fermentation feedstock or in chemical synthesis. As used herein, the term “cellulosic biomass” refers to living or dead biological material that contains a cellulose substrate, such as, for example, lignocellulose, hemicellulose, lignin, and the like. Therefore, the present invention provides a method of convening a biomass substrate to a degradation product, the method comprising contacting a culture medium or cell lysate containing a biomass degradation polypeptide according to the invention, with the biomass substrate under conditions suitable for the production of the degradation product. The degradation product can be an end product such as a soluble sugar, or a product that undergoes further enzymatic conversion to an end product such as a soluble sugar. For example, a biomass degradation enzyme of the invention may participate in a reaction that makes the cellulosic substrate more susceptible to hydrolysis so that the substrate is more readily hydrolyzed to fermentable sugars, such as glucose, cellobiose, xylose, xylulose, arabinose, mannose, galactose, and/or soluble oligosaccharides. The cellulosic substrate can be contacted with a composition, culture medium or cell lysate containing biomass degradation polypeptide of Tables 1, 2, 3, or 4 (and optionally other enzymes involved in breaking down cellulosic biomass) under conditions suitable for the production of a biomass degradation product. In some embodiments, the contacting step may involve contacting the biomass with a composition, culture medium, or cell lysate containing an accessory protein such as an esterase, laccase. etc. set forth in Tables 1, 2, 3, or 4. In some embodiments, the contacting step may involve contacting the biomass with a composition, culture medium, or cell lysate containing a glycosyl hydrolase set forth in Tables 1, 2, 3, or 4.

Thus, the present invention provides a method for producing a biomass degradation product by (a) providing a cellulosic biomass; and (b) contacting the biomass with at least one biomass degradation polypeptide that has an amino acid sequence set forth in Tables 1, 2, 3, or 4 under conditions sufficient to form a reaction mixture for converting the biomass to a degradation product such as a soluble carbohydrate, or a product that is more readily hydrolyzed to a soluble carbohydrate. The cellulose degradation polypeptide may be used in such methods in either isolated form or as part of a composition, such as any of those described herein. The biomass degradation polypeptide may also be provided in cell culturing media or in a cell lysate. For example, after producing a biomass degradation enzyme of the invention by culturing a host cell transformed with a biomass degradation polynucleotide or vector of the present invention, the enzyme need not be isolated from the culture medium (i.e., if the enzyme is secreted into the culture medium) or cell lysate (i.e., if the enzyme is not secreted into the culture medium) or used in a purified form to be useful. Any composition, cell culture medium, or cell lysate containing a biomass degradation enzyme of the present invention may be suitable for use in methods to degrade cellulosic biomass. Therefore, the present invention further provides a method for producing a degradation product of cellulosic biomass, such as a soluble sugar, a de-esterified cellulose biomass, etc. by: (a) providing a cellulosic biomass; and (b) contacting the biomass with a culture medium or cell lysate or composition comprising at least one biomass degradation polypeptide having an amino acid sequence of Tables 1, 2, 3, or 4 e.g., a glycoside hydrolase of Tables 1, 2, 3, or 4, under conditions sufficient to form a reaction mixture for converting the cellulosic biomass to the degradation product.

In some embodiments, the biomass includes cellulosic substrates including but not limited to, wood, wood pulp, paper pulp, corn stover, corn fiber, rice, paper and pulp processing waste, woody or herbaceous plants, fruit or vegetable pulp, distillers grain, grasses, rice hulls, wheat straw, cotton, hemp, flax, sisal, corn cobs, sugar cane bagasse, switch grass and mixtures thereof. The biomass may optionally be pretreated to increase the susceptibility of cellulose to hydrolysis using methods known in the art such as chemical, physical and biological pretreatments (e.g., steam explosion, pulping, grinding, acid hydrolysis, solvent exposure, and the like, as well as combinations thereof).

Soluble sugars produced by the methods of the present invention may be used to produce an alcohol (such as, for example, ethanol, butanol, and the like). The present invention therefore provides a method of producing an alcohol, where the method comprises (a) providing a soluble sugar produced using a biomass degradation polypeptide of the present invention in the methods described supra; (b) contacting the soluble sugar with a fermenting microorganism to produce the alcohol or other metabolic product; and (c) recovering the alcohol or other metabolic product.

In some embodiments, a biomass degradation polypeptide of the present invention, or composition, cell culture medium, or cell lysate containing the polypeptide, may be used to catalyze the hydrolysis of a biomass substrate to a soluble sugar in the presence of a fermenting microorganism such as a yeast (e.g., Saccharomyces sp., such as, for example, S. cerevisiae, Zymomonas sp., E. coli, Pichia sp., and the like) or other C5 or C6 fermenting microorganisms that are well known in the art, to produce an end-product such as ethanol. In this simultaneous saccharification and fermentation (SSF) process the soluble sugars (e.g., glucose and/or xylose) are removed from the system by the fermentation process.

The soluble sugars produced by the use of a biomass degradation polypeptide of the present invention may also be used in the production of other end-products, such as, for example, acetone, an amino acid (e.g., glycine, lysine, and the like), an organic acid (e.g., lactic acid, and the like), glycerol, a diol (e.g., 1,3 propanediol, butanediol, and the like) and animal feeds.

One of skill in the art will readily appreciate that biomass degradation polypeptide compositions of the present invention may be used in the form of an aqueous solution or a solid concentrate. When aqueous solutions are employed, the solution can easily be diluted to allow accurate concentrations. A concentrate can be in any form recognized in the art including, for example, liquids, emulsions, suspensions, gel, pastes, granules, powders, an agglomerate, a solid disk, as well as other forms that are well known in the art. Other materials can also be used with or included in the enzyme composition of the present invention as desired, including stones, pumice, fillers, solvents, enzyme activators, and anti-redeposition agents depending on the intended use of the composition.

The foregoing and other aspects of the invention may be better understood in connection with the following non-limiting examples.

### VIII. Examples

### Example 1

### Cellulase Induction Experiments

This example identified genes that were differently expressed or secreted by a Myceliophthora thermophila strain upon induction with a microcrystalline cellulose preparation or incubation with a wheat straw biomass-derived sugar hydrolysate. In this experiment, 2×150 mL of cultures were inoculated in YPD media at 35° C. (250 rpm). After 90 hours, the cultures were harvested and washed. Then 3×50 mL of resulting cultures were started in M56 fermentation media containing 4% Avicel or wheat straw extract. Samples (1.5 mL) were collected at 0, 0.25, 0.5, 1, 2, 4, 8, 24, and 48 hours and cDNA was prepared from the cell samples. The cDNA preparations were labeled and hybridized to Agilent arrays following standard protocols. The arrays were washed and scanned for analysis. Genes over-expressed in wheat straw hydrolysate; or over-expressed during the time courses were identified and genes were selected based on a function of interest and/or overexpression parameters such as correlation of induction profiles with various cellulases, overexpression in the production strain vs. a wildtype strain, level of overexpression in wheat straw extract at later time points.

### Example 2

### Selection of Additional Genes

Genes were selected based on the following: 1) proteins detected as secreted proteins or protein predicted to be secreted; 2) genes identified from cellulase induction experiments (Example 1); 3) genes with GH domains relevant to biomass degradation, e.g. GH3. GH5. GH6, GH7, GH9, GH12, GH44. GH45. GH74 for cellulases, GH3, GH4, GH5, GH8, GH10, GH11, GH28, GH36, GH39, GH43, GH51, GH52, GH54, GH62, GH67, GH74 for hemicellulases, GH35, GH61 for accessory enzymes, GH4. GH13, GH14, GH15, GH31, GH57, GH63, GH97, GH119, GH122 for amylases; 4) additional gene designations/annotations involved in biomass degradation functions, e.g., endoglucanase, cellobiohydrolase, beta-glucosidase, esterase, endoxylanase, abf, xyloglucanase, pectinase, expansin, alpha-glucuronidase, alpha,beta-xylosidase, beta-galactosidase, mannanase, polysaccharide lyase, arabinase, mannosidase; 5) transcription factors and genes involved in pentose phosphate cycle, signal transduction pathways, secretion pathways, pH/stress response, post-translational modification that improve production and hydrolysis activity; 6) fungal oxidoreductases potentially involved in the degradation of lignin and related aromatic compounds, e.g. laccase, copper oxidase, monooxygenase, and genes with cir1 P450. Cu-oxidase, Glyoxal_oxid, GMC_oxred, Tyrosinase, Cupin_Lipase_GDSL, alcohol_oxidase, copper_amine_oxidase, Abhydrolase type of domains.

While the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

TABLE 1

| | | | | Column 5 |
|---|---|---|---|---|
| | | | Column 4 | SEQ ID NO |
| | | | SEQ ID NO | (protein |
| | | Column 3 | (protein | sequence, no |
| Column 1 | Column 2 | SEQ ID NO | sequence with | signal |
| V4 Gene Name | Activity # | (cDNA) | signal peptide) | peptide) |
| v4chr1-54196m26 | 8 | 1 | 2 | 3 |
| v4chr3-18239m16 | 8 | 4 | 5 | 6 |
| v4chr2-73043m28 | 10 | 7 | 8 | 9 |
| v4chr4-4572p15 | 10 | 10 | 11 | 12 |
| v4chr6a-25523p13 | 10 | 13 | 14 | 15 |
| v4chr1-30182m27 | 18 | 16 | 17 | 18 |
| v4chr2-1194p18 | 18 | 19 | 20 | 21 |
| v4chr3-6594m16 | 18 | 22 | 23 | 24 |
| v4chr4-33394p15 | 18 | 25 | 26 | 27 |
| v4chr3-11825m27 | 21 | 28 | 29 | 30 |
| v4chr3-34174m29 | 21 | 31 | 32 | 33 |
| v4chr3-50789p30 | 21 | 34 | 35 | 36 |
| v4chr4-6448p10 | 25 | 37 | 38 | 39 |
| v4chr6a-11150m11 | 28 | 40 | 41 | 42 |
| v4chr6b-850p14 | 28 | 43 | 44 | 45 |
| v4chr6a-29793p13 | 28 | 46 | 47 | 48 |
| v4chr2-40227p9 | 29 | 49 | 50 | 51 |
| v4chr2-20586m21 | 36 | 52 | 53 | 54 |
| v4chr5-32868m20 | 36 | 55 | 56 | 57 |
| v4chr4-34944p18 | 36 | 58 | 59 | 60 |
| v4chr1-61131m19 | 38 | 61 | 62 | 63 |
| v4chr3-27037p20 | 38 | 64 | 65 | 66 |
| v4chr5-39651m29 | 39 | 67 | 68 | 69 |
| v4chr4-12709m29 | 39 | 70 | 71 | 72 |
| v4chr7-36312m25 | 39 | 73 | 74 | 75 |
| v4chr1-57343m21 | 39 | 76 | 77 | 78 |
| v4chr5-39576m10 | 39 | 79 | 80 | 81 |
| v4chr6b-1402m7 | 39 | 82 | 83 | 84 |
| v4chr5-9467p19 | 39 | 85 | 86 | 87 |
| v4chr3-32980m12 | 39 | 88 | 89 | 90 |
| v4chr2-14160m24 | 39 | 91 | 92 | 93 |
| v4chr5-14872m8 | 40 | 94 | 95 | 96 |
| v4chr1-6314p14 | 42 | 97 | 98 | 99 |
| v4chr1-61102m14 | 42 | 100 | 101 | 102 |
| v4chr2-23698m19 | 42 | 103 | 104 | 105 |
| v4chr2-60738p15 | 42 | 106 | 107 | 108 |
| v4chr3-1993p13 | 42 | 109 | 110 | 111 |
| v4chr3-2875m14 | 42 | 112 | 113 | 114 |
| v4chr4-46531m19 | 42 | 115 | 116 | 117 |
| v4chr5-43537m13 | 42 | 118 | 119 | 120 |
| v4chr6a-7824m13 | 42 | 121 | 122 | 123 |
| v4chr5-15490p14 | 42 | 124 | 125 | 126 |
| v4chr5-23017m10 | 42 | 127 | 128 | 129 |
| v4chr2-16266p10 | 42 | 130 | 131 | 132 |
| v4chr2-51433m11 | 42 | 133 | 134 | 135 |
| v4chr2-51800m9 | 42 | 136 | 137 | 138 |
| v4chr3-27074m15 | 42 | 139 | 140 | 141 |
| v4chr5-42485p8 | 42 | 142 | 143 | 144 |
| v4chr6a-11947m14 | 42 | 145 | 146 | 147 |
| v4chr1-28415p18 | 42 | 148 | 149 | 150 |
| v4chr3-19798p18 | 42 | 151 | 152 | 153 |
| v4chr6a-19551m28 | 42 | 154 | 155 | 156 |
| v4chr1-33134p10 | 42 | 157 | 158 | 159 |
| v4chr1-51854m13 | 42 | 160 | 161 | 162 |
| v4chr3-19646m8 | 42 | 163 | 164 | 165 |
| v4chr3-23294m7 | 42 | 166 | 167 | 168 |
| v4chr3-30087m12 | 42 | 169 | 170 | 171 |
| v4chr3-43634p8 | 42 | 172 | 173 | 174 |
| v4chr4-10827m13 | 42 | 175 | 176 | 177 |
| v4chr6a-11168p11 | 42 | 178 | 179 | 180 |
| v4chr6a-12368m9 | 42 | 181 | 182 | 183 |
| v4chr6a-18078p8 | 42 | 184 | 185 | 186 |
| v4chr7-36264m7 | 42 | 187 | 188 | 189 |
| v4chr3-18684p9 | 42 | 190 | 191 | 192 |
| v4chr7-36246m10 | 42 | 193 | 194 | 195 |
| v4chr1-17314m10 | 42 | 196 | 197 | 198 |
| v4chr2-16783p13 | 42 | 199 | 200 | 201 |
| v4chr3-18156m13 | 42 | 202 | 203 | 204 |
| v4chr3-19897p15 | 42 | 205 | 206 | 207 |
| v4chr6a-8016p21 | 42 | 208 | 209 | 210 |
| v4chr6b-309m17 | 42 | 211 | 212 | 213 |
| v4chr7-29412p13 | 42 | 214 | 215 | 216 |
| v4chr7-7921m7 | 42 | 217 | 218 | 219 |
| v4chr2-61184p17 | 42 | 220 | 221 | 222 |
| v4chr2-75425m8 | 42 | 223 | 224 | 225 |
| v4chr4-16641p21 | 42 | 226 | 277 | 228 |
| v4chr4-49590m7 | 42 | 229 | 230 | 231 |
| v4chr5-1414p17 | 42 | 232 | 233 | 234 |
| v4chr7-23480p21 | 42 | 235 | 236 | 237 |
| v4chr1-45969p13 | 42 | 238 | 239 | 240 |
| v4chr2-69550p2 | 42 | 241 | 242 | 243 |
| v4chr3-16249p19 | 42 | 244 | 245 | 246 |
| v4chr5-13441m15 | 42 | 247 | 248 | 249 |
| v4chr3-2130m7 | 42 | 250 | 251 | 252 |
| v4chr4-44326p8 | 42 | 253 | 254 | 255 |
| v4chr3-33604p8 | 10, 42 | 256 | 257 | 258 |
| v4chr2-58146p8 | 12, 13 | 259 | 260 | 261 |
| v4chr3-1974p9 | 12, 13 | 262 | 263 | 264 |
| v4chr5-40741p11 | 12, 13 | 265 | 266 | 267 |
| v4chr6a-34208p7 | 12, 13 | 268 | 269 | 270 |
| v4chr7-7143m7 | 12, 13 | 271 | 272 | 273 |
| v4chr1-42827p7 | 12, 13 | 274 | 275 | 276 |
| v4chr3-12198m16 | 15, 19 | 277 | 278 | 279 |
| v4chr6a-12299m16 | 15, 19 | 280 | 281 | 282 |
| v4chr1-45858p9 | 17, 23 | 283 | 284 | 285 |
| v4chr4-44244p12 | 17, 23 | 286 | 287 | 288 |
| v4chr5-6640p9 | 20, 23 | 289 | 290 | 291 |
| v4chr7-23790m22 | 22, 23 | 292 | 293 | 294 |
| v4chr2-18381p22 | 23, 29 | 295 | 296 | 297 |
| v4chr3-54200m17 | 23, 29 | 298 | 299 | 300 |
| v4chr3-813m12 | 23, 29 | 301 | 302 | 303 |
| v4chr4-8869m13 | 23, 29 | 304 | 305 | 306 |
| v4chr5-38617m13 | 23, 29 | 307 | 308 | 309 |
| v4chr2-66290m20 | 25, 39 | 310 | 311 | 312 |
| v4chr6b-7438p10 | 26, 27 | 313 | 314 | 315 |
| v4chr7-8477p12 | 26, 27 | 316 | 317 | 318 |
| v4chr6a-11852p7 | 26, 27 | 319 | 320 | 321 |
| v4chr4-4420m17 | 3, 4, 7, 9 | 322 | 323 | 324 |
| v4chr5-270m11 | 3, 4, 7, 9 | 325 | 326 | 327 |
| v4chr5-279p12 | 3, 4, 7, 9 | 328 | 329 | 330 |
| v4chr4-1883m23 | 3, 4, 7, 9, 16 | 331 | 332 | 333 |
| v4chr7-17283p10 | 3, 4, 7, 9, 16 | 334 | 335 | 336 |
| v4chr1-259m24 | 3, 4, 7, 9, 16 | 337 | 338 | 339 |
| v4chr4-1983m23 | 3, 4, 7, 9, 16 | 340 | 341 | 342 |
| v4chr5-22719p25 | 3, 4, 7, 9, 36 | 343 | 344 | 345 |
| v4chr6a-10875p9 | 3, 4, 9 | 346 | 347 | 348 |
| v4chr1-22293m11 | 3, 4, 9 | 349 | 350 | 351 |
| v4chr6b-11049m8 | 34, 41 | 352 | 353 | 354 |
| v4chr2-16972p12 | 36, 37 | 355 | 356 | 357 |
| v4chr5-3703m24 | 36, 45 | 358 | 359 | 360 |
| v4chr6b-12886p23 | 36, 45 | 361 | 362 | 363 |
| v4chr4-293m24 | 38, 39 | 364 | 365 | 366 |
| v4chr1-28579p15 | 5, 12, 13, 17, 23, 29, 42 | 367 | 368 | 369 |
| v4chr5-22308m12 | 5, 17, 23 | 370 | 371 | 372 |
| v4chr6b-14222p17 | 5, 17, 23 | 373 | 374 | 375 |
| v4chr1-16223p10 | 5, 17, 23 | 376 | 377 | 378 |
| v4chr5-45043m10 | 5, 17, 23 | 379 | 380 | 381 |
| v4chr6a-34292m9 | 5, 17, 23 | 382 | 383 | 384 |
| v4chr1-2303p9 | 5, 17, 23, 29 | 385 | 386 | 387 |
| v4chr1-60519p12 | 5, 17, 23, 31 | 388 | 389 | 390 |
| v4chr4-34206m11 | 5, 17, 23, 31 | 391 | 392 | 393 |
| v4chr1-21106m12 | 5, 8, 12, 13, 17, 23, 29 | 394 | 395 | 396 |
| v4chr7-169p13 | 5, 8, 12, 13, 17, 23, 29 | 397 | 398 | 399 |
| v4chr4-31038m15 | 7, 16, 23 | 400 | 401 | 402 |

TABLE 2

| | | Column 3 | Column 4 |
|---|---|---|---|
| Column 1 | Column 2 | SEQ ID NO | SEQ ID NO |
| V4 Gene Name | Activity # | (cDNA) | (protein) |
| v4chr2-25393m33 | 8 | 403 | 404 |
| v4chr2-11466m13 | 10 | 405 | 406 |
| v4chr2-36725m33 | 18 | 407 | 408 |
| v4chr7-17659p36 | 21 | 409 | 410 |
| v4chr5-12936p20 | 21 | 411 | 412 |
| v4chr1-42596p46 | 35 | 413 | 414 |
| v4chr6a-20419m22 | 36 | 415 | 416 |
| v4chr5-3942p25 | 38 | 417 | 418 |
| v4chr6b-13880p10 | 38 | 419 | 420 |
| v4chr4-7403m7 | 38 | 421 | 422 |
| v4chr7-28444m14 | 38 | 423 | 424 |
| v4chr5-35032p8 | 39 | 425 | 426 |
| v4chr6a-32476m12 | 39 | 427 | 428 |
| v4chr6a-27714p17 | 39 | 429 | 430 |
| v4chr3-39854p15 | 40 | 431 | 432 |
| v4chr1-39559m15 | 42 | 433 | 434 |
| v4chr3-17051p18 | 42 | 435 | 436 |
| v4chr4-47338m22 | 42 | 437 | 438 |
| v4chr7-9702m21 | 42 | 439 | 440 |
| v4chr5-7342p13 | 42 | 441 | 442 |
| v4chr6a-2573m13 | 42 | 443 | 444 |
| v4chr5-37116m11 | 42 | 445 | 446 |
| v4chr2-17230m25 | 42 | 447 | 448 |
| v4chr1-37598m36 | 42 | 449 | 450 |
| v4chr2-24292p15 | 42 | 451 | 452 |
| v4chr2-61903p19 | 42 | 453 | 454 |
| v4chr2-65345m23 | 42 | 455 | 456 |
| v4chr2-6752m16 | 42 | 457 | 458 |
| v4chr2-72695m22 | 42 | 459 | 460 |
| v4chr2-77127p22 | 42 | 461 | 462 |
| v4chr2-8686p26 | 42 | 463 | 464 |
| v4chr3-22012p19 | 42 | 465 | 466 |
| v4chr3-3127m9 | 42 | 467 | 468 |
| v4chr3-38467m33 | 42 | 469 | 470 |
| v4chr3-40560p11 | 42 | 471 | 472 |
| v4chr3-42190p15 | 42 | 473 | 474 |
| v4chr3-42218p20 | 42 | 475 | 476 |
| v4chr3-54225p15 | 42 | 477 | 478 |
| v4chr3-749m3 | 42 | 479 | 480 |
| v4chr4-20339p30 | 42 | 481 | 482 |
| v4chr4-32652p28 | 42 | 483 | 484 |
| v4chr5-31523m9 | 42 | 485 | 486 |
| v4chr5-32373p12 | 42 | 487 | 488 |
| v4chr5-37947p16 | 42 | 489 | 490 |
| v4chr5-48185p9 | 42 | 491 | 492 |
| v4chr6a-17391p2 | 42 | 493 | 494 |
| v4chr7-18866m38 | 42 | 495 | 496 |
| v4chr7-8999m14 | 42 | 497 | 498 |
| v4chr1-12713p16 | 42 | 499 | 500 |
| v4chr1-56580p10 | 42 | 501 | 502 |
| v4chr1-58887p11 | 42 | 503 | 504 |
| v4chr2-50714m27 | 42 | 505 | 506 |
| v4chr2-69554p20 | 42 | 507 | 508 |
| v4chr3-17149m10 | 42 | 509 | 510 |
| v4chr4-30942p10 | 42 | 511 | 512 |
| v4chr4-6224m23 | 42 | 513 | 514 |
| v4chr5-20717p29 | 42 | 515 | 516 |
| v4chr6a-1402m24 | 42 | 517 | 518 |
| v4chr2-17834p17 | 42 | 519 | 520 |
| v4chr2-62662m13 | 42 | 521 | 522 |
| v4chr4-39821p9 | 42 | 523 | 524 |
| v4chr5-12218m5 | 42 | 525 | 526 |
| v4chr6a-16604p12 | 42 | 527 | 528 |
| v4chr5-41364p11 | 42 | 529 | 530 |
| v4chr3-8836p3 | 12, 13 | 531 | 532 |
| v4chr1-267p30 | 21, 44 | 533 | 534 |
| v4chr4-6158p25 | 21, 44 | 535 | 536 |
| v4chr4-323m16 | 3, 4, 7, 9 | 537 | 538 |
| v4chr6b-6880m9 | 3, 4, 7, 9 | 539 | 540 |
| v4chr3-4714m16 | 3, 4, 7, 9, 16 | 541 | 542 |
| v4chr6b-1577m30 | 3, 4, 7, 9, 16 | 543 | 544 |
| v4chr4-49475m22 | 36, 37 | 545 | 546 |
| v4chr6a-8375p26 | 36, 39 | 547 | 548 |
| v4chr6b-10059m32 | 36, 39 | 549 | 550 |
| v4chr2-61708p22 | 36, 45 | 551 | 552 |
| v4chr4-39108p15 | 5, 12, 13, 16, | 553 | 554 |
| | 17, 23, 29 | | |
| v4chr6a-19658m23 | 5, 12, 13, 17, 23, 29 | 555 | 556 |
| v4chr5-34806m26 | 8, 33 | 557 | 558 |

TABLE 3

| | | | | Column 5 |
|---|---|---|---|---|
| | | | Column 4 | SEQ ID NO |
| | | Column 3 | SEQ ID NO | (protein |
| | | SEQ ID | (protein | sequence, no |
| Column 1 | Column 2 | NO | sequence with | signal |
| V4 Gene Name | Activity # | (cDNA) | signal peptide) | peptide) |
| v4chr6a-5087p26 | 6 | 559 | 560 | 561 |
| v4chr5-46937m26 | 8 | 562 | 563 | 564 |
| v4s103-1p12 | 8 | 565 | 566 | 567 |
| v4chr1-14031m3 | 10 | 568 | 569 | 570 |
| v4chr5-47909m12 | 10 | 571 | 572 | 573 |
| v4chr6b-15681p9 | 10 | 574 | 575 | 576 |
| v4chr7-152p8 | 10 | 577 | 578 | 579 |
| v4chr6a-2053p12 | 10 | 580 | 581 | 582 |
| v4chr4-42966m7 | 10 | 583 | 584 | 585 |
| v4chr5-47972p44 | 10 | 586 | 587 | 588 |
| v4chr7-15635m14 | 10 | 589 | 590 | 591 |
| v4chr5-27445m29 | 18 | 592 | 593 | 594 |
| v4chr1-11300p13 | 18 | 595 | 596 | 597 |
| v4chr1-6188p11 | 18 | 598 | 599 | 600 |
| v4chr3-12801m15 | 18 | 601 | 602 | 603 |
| v4chr6a-23743p16 | 18 | 604 | 605 | 606 |
| v4s151-41m13 | 18 | 607 | 608 | 609 |
| v4chr1-11374p13 | 18 | 610 | 611 | 612 |
| v4chr2-22055p33 | 21 | 613 | 614 | 615 |
| v4chr2-56875p13 | 23 | 616 | 617 | 618 |
| v4chr6b-14138p24 | 23 | 619 | 620 | 621 |
| v4chr7-16675p14 | 25 | 622 | 623 | 624 |
| v4chr1-1553m8 | 28 | 625 | 626 | 627 |
| v4chr2-42614m11 | 31 | 628 | 629 | 630 |
| v4chr5-33720m17 | 32 | 631 | 632 | 633 |
| v4chr6a-22593m17 | 32 | 634 | 635 | 636 |
| v4chr7-2448p16 | 32 | 637 | 638 | 639 |
| v4chr2-23202p17 | 33 | 640 | 641 | 642 |
| v4chr2-68710p16 | 35 | 643 | 644 | 645 |
| v4chr3-1420m15 | 35 | 646 | 647 | 648 |
| v4chr5-45534m16 | 35 | 649 | 650 | 651 |
| v4chr1-22157m14 | 35 | 652 | 653 | 654 |
| v4chr3-2834p10 | 35 | 655 | 656 | 657 |
| v4chr4-32238p11 | 35 | 658 | 659 | 660 |
| v4chr7-1388p29 | 35 | 661 | 662 | 663 |
| v4chr7-16605m18 | 35 | 664 | 665 | 666 |
| v4chr3-11441p19 | 35 | 667 | 668 | 669 |
| v4chr6a-2108p13 | 35 | 670 | 671 | 672 |
| v4chr3-26611p21 | 35 | 673 | 674 | 675 |
| v4chr5-42029m18 | 35 | 676 | 677 | 678 |
| v4chr7-2889p16 | 35 | 679 | 680 | 681 |
| v4chr6a-36911m16 | 35 | 682 | 683 | 684 |
| v4chr3-21761p16 | 35 | 685 | 686 | 687 |
| v4chr6a-18968p13 | 35 | 688 | 689 | 690 |
| v4chr5-7830p28 | 35 | 691 | 692 | 693 |
| v4chr2-24527p10 | 35 | 694 | 695 | 696 |
| v4chr1-48293m21 | 36 | 697 | 698 | 699 |
| v4chr4-3987m21 | 36 | 700 | 701 | 702 |
| v4chr4-49300m19 | 36 | 703 | 704 | 705 |
| v4chr3-49292m21 | 36 | 706 | 707 | 708 |
| v4chr2-17550m19 | 36 | 709 | 710 | 711 |
| v4chr6b-382m12 | 36 | 712 | 713 | 714 |
| v4chr4-44885m7 | 36 | 715 | 716 | 717 |
| v4chr1-18546p13 | 36 | 718 | 719 | 720 |
| v4chr1-57459m14 | 36 | 721 | 722 | 723 |
| v4chr3-16285p8 | 36 | 724 | 775 | 726 |
| v4chr3-22337m20 | 36 | 727 | 728 | 729 |
| v4chr3-23353p15 | 36 | 730 | 731 | 732 |
| v4chr4-1148p21 | 36 | 733 | 734 | 735 |
| v4chr4-1262p18 | 36 | 736 | 737 | 738 |
| v4chr5-44551p4 | 36 | 739 | 740 | 741 |
| v4chr6a-5405m18 | 36 | 742 | 743 | 744 |
| v4chr2-15086m19 | 38 | 745 | 746 | 747 |
| v4chr2-24247p11 | 38 | 748 | 749 | 750 |
| v4chr2-51729p12 | 38 | 751 | 752 | 753 |
| v4chr4-13630m11 | 38 | 754 | 755 | 756 |
| v4chr4-1406p18 | 38 | 757 | 758 | 759 |
| v4chr5-15180m18 | 38 | 760 | 761 | 762 |
| v4chr5-29634p19 | 38 | 763 | 764 | 765 |
| v4chr5-44803m21 | 38 | 766 | 767 | 768 |
| v4chr5-8009p33 | 38 | 769 | 770 | 771 |
| v4chr6a-15077p7 | 38 | 772 | 773 | 774 |
| v4chr6a-21464m17 | 38 | 775 | 776 | 777 |
| v4chr6a-21543p19 | 38 | 778 | 779 | 780 |
| v4chr6a-32779p2 | 38 | 781 | 782 | 783 |
| v4chr7-25280m14 | 38 | 784 | 785 | 786 |
| v4chr1-57507m21 | 38 | 787 | 788 | 789 |
| v4chr2-39219p21 | 38 | 790 | 791 | 792 |
| v4chr3-53190m19 | 38 | 793 | 794 | 795 |
| v4chr3-8166m21 | 38 | 796 | 797 | 798 |
| v4chr5-15070p20 | 38 | 799 | 800 | 801 |
| v4chr5-4838p16 | 38 | 802 | 803 | 804 |
| v4chr5-7275m18 | 38 | 805 | 806 | 807 |
| v4chr6a-21517p7 | 38 | 808 | 809 | 810 |
| v4chr6a-29731p20 | 38 | 811 | 812 | 813 |
| v4chr6a-31800m18 | 38 | 814 | 815 | 816 |
| v4chr6a-35660p11 | 38 | 817 | 818 | 819 |
| v4chr6a-4983m19 | 38 | 820 | 821 | 822 |
| v4chr6b-14184p21 | 38 | 823 | 824 | 825 |
| v4chr5-16338m18 | 38 | 826 | 827 | 828 |
| v4chr6b-8529p17 | 38 | 829 | 830 | 831 |
| v4chr1-6618p14 | 38 | 832 | 833 | 834 |
| v4chr1-35264p17 | 38 | 835 | 836 | 837 |
| v4chr2-21018p20 | 38 | 838 | 839 | 840 |
| v4chr2-23085p25 | 38 | 841 | 842 | 841 |
| v4chr2-63927m8 | 38 | 844 | 845 | 846 |
| v4chr3-2331m11 | 38 | 847 | 848 | 849 |
| v4chr5-13619p7 | 38 | 850 | 851 | 852 |
| v4chr5-25149p5 | 38 | 853 | 854 | 855 |
| v4chr2-64098m27 | 38 | 856 | 857 | 858 |
| v4chr1-48612m9 | 38 | 859 | 860 | 861 |
| v4chr2-68594p3 | 38 | 862 | 863 | 864 |
| v4chr2-75551m12 | 38 | 865 | 866 | 867 |
| v4chr3-4899m5 | 38 | 868 | 869 | 870 |
| v4chr2-28764p7 | 38 | 871 | 872 | 873 |
| v4chr4-41898p5 | 39 | 874 | 875 | 876 |
| v4chr7-40174p20 | 39 | 877 | 878 | 879 |
| v4chr6b-8441p8 | 39 | 880 | 881 | 882 |
| v4chr3-19463p9 | 39 | 883 | 884 | 885 |
| v4chr2-49604p15 | 39 | 886 | 887 | 888 |
| v4chr3-8782p20 | 39 | 889 | 890 | 891 |
| v4chr7-17630p19 | 39 | 892 | 893 | 894 |
| v4chr1-9503p11 | 39 | 895 | 896 | 897 |
| v4chr3-31237p3 | 39 | 898 | 899 | 900 |
| v4chr7-16330m21 | 39 | 901 | 902 | 903 |
| v4chr2-43222m19 | 39 | 904 | 905 | 906 |
| v4chr2-34496m16 | 39 | 907 | 908 | 909 |
| v4chr2-23980p16 | 42 | 910 | 911 | 912 |
| v4chr5-11514p13 | 42 | 913 | 914 | 915 |
| v4chr5-18917p23 | 42 | 916 | 917 | 918 |
| v4chr2-30244m14 | 42 | 919 | 920 | 921 |
| v4chr2-8416m28 | 42 | 922 | 923 | 924 |
| v4chr3-42606p12 | 42 | 925 | 926 | 927 |
| v4chr6a-26935p16 | 42 | 928 | 929 | 930 |
| v4chr7-2107m16 | 42 | 931 | 932 | 933 |
| v4chr7-7263m31 | 42 | 934 | 935 | 936 |
| v4chr1-14013m15 | 42 | 937 | 938 | 939 |
| v4chr1-360m18 | 42 | 940 | 941 | 942 |
| v4chr1-50559p18 | 42 | 943 | 944 | 945 |
| v4chr1-57880m11 | 42 | 946 | 947 | 948 |
| v4chr1-587p12 | 42 | 949 | 950 | 951 |
| v4chr1-9167p12 | 42 | 952 | 953 | 954 |
| v4chr2-14073p8 | 42 | 955 | 956 | 957 |
| v4chr2-21455m13 | 42 | 958 | 959 | 960 |
| v4chr2-75431p11 | 42 | 961 | 962 | 963 |
| v4chr3-41404m15 | 42 | 964 | 965 | 966 |
| v4chr4-1391m13 | 42 | 967 | 968 | 969 |
| v4chr4-579m23 | 42 | 970 | 971 | 972 |
| v4chr5-2301p14 | 42 | 973 | 974 | 975 |
| v4chr5-35126m16 | 42 | 976 | 977 | 978 |
| v4chr5-4747p18 | 42 | 979 | 980 | 981 |
| v4chr5-5934p5 | 42 | 982 | 983 | 984 |
| v4chr5-7429p20 | 42 | 985 | 986 | 987 |
| v4chr6a-25453p10 | 42 | 988 | 989 | 990 |
| v4chr6a-32568m10 | 42 | 991 | 992 | 993 |
| v4chr6b-4863p18 | 42 | 994 | 995 | 996 |
| v4s114-9p8 | 42 | 997 | 998 | 999 |
| v4chr2-24355m19 | 42 | 1000 | 1001 | 1002 |
| v4chr3-21494m15 | 42 | 1003 | 1004 | 1005 |
| v4chr4-27017p22 | 42 | 1006 | 1007 | 1008 |
| v4chr4-37992m17 | 42 | 1009 | 1010 | 1011 |
| v4chr4-3957m20 | 42 | 1012 | 1013 | 1014 |
| v4chr4-4030m18 | 42 | 1015 | 1016 | 1017 |
| v4chr4-6637m20 | 42 | 1018 | 1019 | 1020 |
| v4chr4-8254m19 | 42 | 1021 | 1022 | 1023 |
| v4chr7-8359m17 | 42 | 1024 | 1025 | 1026 |
| v4chr3-23968p10 | 42 | 1027 | 1028 | 1029 |
| v4chr4-45657m19 | 42 | 1030 | 1031 | 1032 |
| v4chr1-11419p10 | 42 | 1033 | 1034 | 1035 |
| v4chr3-19448p11 | 42 | 1036 | 1037 | 1038 |
| v4chr3-40195p14 | 42 | 1039 | 1040 | 1041 |
| v4chr5-1262p7 | 42 | 1042 | 1043 | 1044 |
| v4chr5-7902p6 | 42 | 1045 | 1046 | 1047 |
| v4chr6a-12833p17 | 42 | 1048 | 1049 | 1050 |
| v4chr6a-32898m8 | 42 | 1051 | 1052 | 1053 |
| v4chr6a-911p10 | 42 | 1054 | 1055 | 1056 |
| v4chr7-9489m24 | 42 | 1057 | 1058 | 1059 |
| v4chr2-40000p19 | 42 | 1060 | 1061 | 1062 |
| v4chr5-21253m19 | 42 | 1063 | 1064 | 1065 |
| v4chr7-1537m19 | 42 | 1066 | 1067 | 1068 |
| v4chr5-39698m9 | 42 | 1069 | 1070 | 1071 |
| v4chr6a-29703p9 | 42 | 1072 | 1073 | 1074 |
| v4chr6b-10282m10 | 42 | 1075 | 1076 | 1077 |
| v4chr1-16655m13 | 42 | 1078 | 1079 | 1080 |
| v4chr1-54416m10 | 42 | 1081 | 1082 | 1083 |
| v4chr2-58041p14 | 42 | 1084 | 1085 | 1086 |
| v4chr1-36840m3 | 42 | 1087 | 1088 | 1089 |
| v4chr2-12801m25 | 42 | 1090 | 1091 | 1092 |
| v4chr2-55602p5 | 42 | 1093 | 1094 | 1095 |
| v4chr5-24409m24 | 42 | 1096 | 1097 | 1098 |
| v4chr6a-922p7 | 42 | 1099 | 1100 | 1101 |
| v4chr6b-13435p10 | 42 | 1102 | 1103 | 1104 |
| v4chr1-24905m10 | 42 | 1105 | 1106 | 1107 |
| v4chr3-36282p12 | 42 | 1108 | 1109 | 1110 |
| v4chr5-9543m7 | 42 | 1111 | 1112 | 1113 |
| v4chr3-2762p9 | 42 | 1114 | 1115 | 1116 |
| v4chr1-679m13 | 42 | 1117 | 1118 | 1119 |
| v4chr1-16176m10 | 42 | 1120 | 1121 | 1122 |
| v4chr2-156p21 | 42 | 1123 | 1124 | 1125 |
| v4chr6b-13426m12 | 42 | 1126 | 1127 | 1128 |
| v4chr1-11242p10 | 42 | 1129 | 1130 | 1131 |
| v4chr1-11870m2 | 42 | 1132 | 1133 | 1134 |
| v4chr1-16159p6 | 42 | 1135 | 1136 | 1137 |
| v4chr1-18392p15 | 42 | 1138 | 1139 | 1140 |
| v4chr1-21382m14 | 42 | 1141 | 1142 | 1143 |
| v4chr1-21560p14 | 42 | 1144 | 1145 | 1146 |
| v4chr1-2905m25 | 42 | 1147 | 1148 | 1149 |
| v4chr1-30199p7 | 42 | 1150 | 1151 | 1152 |
| v4chr1-30249m14 | 42 | 1153 | 1154 | 1155 |
| v4chr1-44534m4 | 42 | 1156 | 1157 | 1158 |
| v4chr1-46847p12 | 42 | 1159 | 1160 | 1161 |
| v4chr1-49429m9 | 42 | 1162 | 1163 | 1164 |
| v4chr1-51362p10 | 42 | 1165 | 1166 | 1167 |
| v4chr1-51541m11 | 42 | 1168 | 1169 | 1170 |
| v4chr1-5302p12 | 42 | 1171 | 1172 | 1173 |
| v4chr1-54396m8 | 42 | 1174 | 1175 | 1176 |
| v4chr1-58020p27 | 42 | 1177 | 1178 | 1179 |
| v4chr1-61283m25 | 42 | 1180 | 1181 | 1182 |
| v4chr1-8088p2 | 42 | 1183 | 1184 | 1185 |
| v4chr1-8271m4 | 42 | 1186 | 1187 | 1188 |
| v4chr2-11396m14 | 42 | 1189 | 1190 | 1191 |
| v4chr2-1483p17 | 42 | 1192 | 1193 | 1194 |
| v4chr2-15130m20 | 42 | 1195 | 1196 | 1197 |
| v4chr2-15434p22 | 42 | 1198 | 1199 | 1200 |
| v4chr2-17391p5 | 42 | 1201 | 1202 | 1203 |
| v4chr2-19271m7 | 42 | 1204 | 1205 | 1206 |
| v4chr2-19317p19 | 42 | 1207 | 1208 | 1209 |
| v4chr2-20249p24 | 42 | 1210 | 1211 | 1212 |
| v4chr2-30610m9 | 42 | 1213 | 1214 | 1215 |
| v4chr2-31227p4 | 42 | 1216 | 1217 | 1218 |
| v4chr2-31261p27 | 42 | 1219 | 1220 | 1221 |
| v4chr2-3127m3 | 42 | 1222 | 1223 | 1224 |
| v4chr2-31365m5 | 42 | 1225 | 1226 | 1227 |
| v4chr2-3175m22 | 42 | 1228 | 1229 | 1230 |
| v4chr2-39722m19 | 42 | 1231 | 1232 | 1233 |
| v4chr2-43829m53 | 42 | 1234 | 1235 | 1236 |
| v4chr2-50840p46 | 42 | 1237 | 1238 | 1239 |
| v4chr2-54387m9 | 42 | 1240 | 1241 | 1242 |
| v4chr2-57360p3 | 42 | 1243 | 1244 | 1245 |
| v4chr2-589m14 | 42 | 1246 | 1247 | 1248 |
| v4chr2-65874p14 | 42 | 1249 | 1250 | 1251 |
| v4chr2-69530m16 | 42 | 1252 | 1253 | 1254 |
| v4chr2-73210p74 | 42 | 1255 | 1256 | 1257 |
| v4chr2-75103p19 | 42 | 1258 | 1259 | 1260 |
| v4chr2-76081p16 | 42 | 1261 | 1262 | 1263 |
| v4chr2-9537m48 | 42 | 1264 | 1265 | 1266 |
| v4chr3-10248m9 | 42 | 1267 | 1268 | 1269 |
| v4chr3-12122m28 | 42 | 1270 | 1271 | 1272 |
| v4chr3-13330m7 | 42 | 1273 | 1274 | 1275 |
| v4chr3-15119p28 | 42 | 1276 | 1277 | 1278 |
| v4chr3-18085m16 | 42 | 1279 | 1280 | 1281 |
| v4chr3-21367m14 | 42 | 1282 | 1283 | 1284 |
| v4chr3-21396m10 | 42 | 1285 | 1286 | 1287 |
| v4chr3-21453m25 | 42 | 1288 | 1289 | 1290 |
| v4chr3-22101p5 | 42 | 1291 | 1292 | 1293 |
| v4chr3-25456p4 | 42 | 1294 | 1295 | 1296 |
| v4chr3-27352p19 | 42 | 1297 | 1298 | 1299 |
| v4chr3-34237m6 | 42 | 1300 | 1301 | 1302 |
| v4chr3-3901p8 | 42 | 1303 | 1304 | 1305 |
| v4chr3-41315p26 | 42 | 1306 | 1307 | 1308 |
| v4chr3-49945m7 | 42 | 1309 | 1310 | 1311 |
| v4chr3-50196m35 | 42 | 1312 | 1313 | 1314 |
| v4chr3-8985m11 | 42 | 1315 | 1316 | 1317 |
| v4chr4-10771p12 | 42 | 1318 | 1319 | 1320 |
| v4chr4-14223m5 | 42 | 1321 | 1322 | 1323 |
| v4chr4-17965m20 | 42 | 1324 | 1325 | 1326 |
| v4chr4-21113m30 | 42 | 1327 | 1328 | 1329 |
| v4chr4-24821m2 | 42 | 1330 | 1331 | 1332 |
| v4chr4-25108m9 | 42 | 1333 | 1334 | 1335 |
| v4chr4-30930m12 | 42 | 1336 | 1337 | 1338 |
| v4chr4-32722p13 | 42 | 1339 | 1340 | 1341 |
| v4chr4-33722m5 | 42 | 1342 | 1343 | 1344 |
| v4chr4-34210p2 | 42 | 1345 | 1346 | 1347 |
| v4chr4-40062p19 | 42 | 1348 | 1349 | 1350 |
| v4chr4-41357p24 | 42 | 1351 | 1352 | 1353 |
| v4chr4-42419m8 | 42 | 1354 | 1355 | 1356 |
| v4chr4-45897p8 | 42 | 1357 | 1358 | 1359 |
| v4chr4-49176p10 | 42 | 1360 | 1361 | 1362 |
| v4chr4-49352p17 | 42 | 1363 | 1364 | 1365 |
| v4chr4-7328m11 | 42 | 1366 | 1367 | 1368 |
| v4chr5-10039p4 | 42 | 1369 | 1370 | 1371 |
| v4chr5-14756p19 | 42 | 1372 | 1373 | 1374 |
| v4chr5-15913p14 | 42 | 1375 | 1376 | 1377 |
| v4chr5-16072p4 | 42 | 1378 | 1379 | 1380 |
| v4chr5-17580p36 | 42 | 1381 | 1382 | 1383 |
| v4chr5-21093p40 | 42 | 1384 | 1385 | 1386 |
| v4chr5-23109m16 | 42 | 1387 | 1388 | 1389 |
| v4chr5-23164p25 | 42 | 1390 | 1391 | 1392 |
| v4chr5-24370p12 | 42 | 1393 | 1394 | 1395 |
| v4chr5-25106p35 | 42 | 1396 | 1397 | 1398 |
| v4chr5-29257m28 | 42 | 1399 | 1400 | 1401 |
| v4chr5-36518p9 | 42 | 1402 | 1403 | 1404 |
| v4chr5-37995m22 | 42 | 1405 | 1406 | 1407 |
| v4chr5-39252m15 | 42 | 1408 | 1409 | 1410 |
| v4chr5-39288p12 | 42 | 1411 | 1412 | 1413 |
| v4chr5-48048m5 | 42 | 1414 | 1415 | 1416 |
| v4chr6a-10450m6 | 42 | 1417 | 1418 | 1419 |
| v4chr6a-14429m11 | 42 | 1420 | 1421 | 1422 |
| v4chr6a-21121p17 | 42 | 1423 | 1424 | 1425 |
| v4chr6a-24484m21 | 42 | 1426 | 1427 | 1428 |
| v4chr6a-25193p5 | 42 | 1429 | 1430 | 1431 |
| v4chr6a-29191m45 | 42 | 1432 | 1433 | 1434 |
| v4chr6a-33318p2 | 42 | 1435 | 1436 | 1437 |
| v4chr6a-3406p11 | 42 | 1438 | 1439 | 1440 |
| v4chr6a-36501p13 | 42 | 1441 | 1442 | 1443 |
| v4chr6a-4194m4 | 42 | 1444 | 1445 | 1446 |
| v4chr6a-7588m11 | 42 | 1447 | 1448 | 1449 |
| v4chr6b-11724m12 | 42 | 1450 | 1451 | 1452 |
| v4chr6b-13729p25 | 42 | 1453 | 1454 | 1455 |
| v4chr6b-14338m16 | 42 | 1456 | 1457 | 1458 |
| v4chr6b-15954p6 | 42 | 1459 | 1460 | 1461 |
| v4chr6b-1892m10 | 42 | 1462 | 1463 | 1464 |
| v4chr6b-1924m7 | 42 | 1465 | 1466 | 1467 |
| v4chr6b-5322m18 | 42 | 1468 | 1469 | 1470 |
| v4chr6b-9661p2 | 42 | 1471 | 1472 | 1473 |
| v4chr7-11210p10 | 42 | 1474 | 1475 | 1476 |
| v4chr7-12177m19 | 42 | 1477 | 1478 | 1479 |
| v4chr7-12561m9 | 42 | 1480 | 1481 | 1482 |
| v4chr7-13728m10 | 42 | 1483 | 1484 | 1485 |
| v4chr7-18717p16 | 42 | 1486 | 1487 | 1488 |
| v4chr7-18773p8 | 42 | 1489 | 1490 | 1491 |
| v4chr7-19900p3 | 42 | 1492 | 1493 | 1494 |
| v4chr7-20048m20 | 42 | 1495 | 1496 | 1497 |
| v4chr7-23846p8 | 42 | 1498 | 1499 | 1500 |
| v4chr7-3037p35 | 42 | 1501 | 1502 | 1503 |
| v4chr7-38382m3 | 42 | 1504 | 1505 | 1506 |
| v4chr7-40004p7 | 42 | 1507 | 1508 | 1509 |
| v4chr7-4500p9 | 42 | 1510 | 1511 | 1512 |
| v4chr7-4640p3 | 42 | 1513 | 1514 | 1515 |
| v4chr7-7946p9 | 42 | 1516 | 1517 | 1518 |
| v4chr7-9934p9 | 42 | 1519 | 1520 | 1521 |
| v4chr1-34708p15 | 42 | 1522 | 1523 | 1524 |
| v4chr1-47727p6 | 42 | 1525 | 1526 | 1527 |
| v4chr2-42988p17 | 42 | 1528 | 1529 | 1530 |
| v4chr2-50815p13 | 42 | 1531 | 1532 | 1533 |
| v4chr4-11767p22 | 42 | 1534 | 1535 | 1536 |
| v4chr4-6404p10 | 42 | 1537 | 1538 | 1539 |
| v4chr4-8415m23 | 42 | 1540 | 1541 | 1542 |
| v4chr5-24084m16 | 42 | 1543 | 1544 | 1545 |
| v4chr5-35313p18 | 42 | 1546 | 1547 | 1548 |
| v4chr5-36767m3 | 42 | 1549 | 1550 | 1551 |
| v4chr5-40287p10 | 42 | 1552 | 1553 | 1554 |
| v4chr5-45193m107 | 42 | 1555 | 1556 | 1557 |
| v4chr6a-29780p7 | 42 | 1558 | 1559 | 1560 |
| v4chr6a-32800p25 | 42 | 1561 | 1562 | 1563 |
| v4chr6a-36704m3 | 42 | 1564 | 1565 | 1566 |
| v4chr7-10463p16 | 42 | 1567 | 1568 | 1569 |
| v4chr7-20489m10 | 42 | 1570 | 1571 | 1572 |
| v4chr7-2058m5 | 42 | 1573 | 1574 | 1575 |
| v4chr2-44551m28 | 42 | 1576 | 1577 | 1578 |
| v4chr3-23343p4 | 42 | 1579 | 1580 | 1581 |
| v4chr5-1565m74 | 42 | 1582 | 1583 | 1584 |
| v4chr5-1590p17 | 42 | 1585 | 1586 | 1587 |
| v4chr5-4533m24 | 42 | 1588 | 1589 | 1590 |
| v4chr6a-25112p10 | 42 | 1591 | 1592 | 1593 |
| v4chr5-16069m14 | 42 | 1594 | 1595 | 1596 |
| v4chr5-41468p11 | 42 | 1597 | 1598 | 1599 |
| v4chr3-45101p22 | 42 | 1600 | 1601 | 1602 |
| v4chr1-33956p8 | 42 | 1603 | 1604 | 1605 |
| v4chr1-5242p23 | 42 | 1606 | 1607 | 1608 |
| v4chr4-44284m18 | 42 | 1609 | 1610 | 1611 |
| v4chr6a-4933p11 | 42 | 1612 | 1613 | 1614 |
| v4chr6b-80p5 | 42 | 1615 | 1616 | 1617 |
| v4chr7-4858m11 | 42 | 1618 | 1619 | 1620 |
| v4chr1-12421p27 | 42 | 1621 | 1622 | 1623 |
| v4chr1-21583m3 | 42 | 1624 | 1625 | 1626 |
| v4chr1-48182m11 | 42 | 1627 | 1628 | 1629 |
| v4chr1-5201m31 | 42 | 1630 | 1631 | 1632 |
| v4chr2-21610m7 | 42 | 1633 | 1634 | 1635 |
| v4chr2-31604p11 | 42 | 1636 | 1637 | 1638 |
| v4chr2-56953p6 | 42 | 1639 | 1640 | 1641 |
| v4chr3-1296p6 | 42 | 1642 | 1643 | 1644 |
| v4chr3-17359p11 | 42 | 1645 | 1646 | 1647 |
| v4chr3-3108m5 | 42 | 1648 | 1649 | 1650 |
| v4chr4-39808p6 | 42 | 1651 | 1652 | 1653 |
| v4chr4-44404m8 | 42 | 1654 | 1655 | 1656 |
| v4chr5-24190m16 | 42 | 1657 | 1658 | 1659 |
| v4chr5-42223m8 | 42 | 1660 | 1661 | 1662 |
| v4chr5-44635m15 | 42 | 1663 | 1664 | 1665 |
| v4chr5-48140p4 | 42 | 1666 | 1667 | 1668 |
| v4chr5-48158m7 | 42 | 1669 | 1670 | 1671 |
| v4chr6a-35639m18 | 42 | 1672 | 1673 | 1674 |
| v4chr6b-11278m7 | 42 | 1675 | 1676 | 1677 |
| v4chr7-28201p11 | 42 | 1678 | 1679 | 1680 |
| v4chr7-4669m14 | 42 | 1681 | 1682 | 1683 |
| v4chr1-9455m22 | 42 | 1684 | 1685 | 1686 |
| v4chr2-24723p15 | 42 | 1687 | 1688 | 1689 |
| v4chr3-27549m11 | 42 | 1690 | 1691 | 1692 |
| v4chr6a-25733m8 | 42 | 1693 | 1694 | 1695 |
| v4chr7-19221p13 | 42 | 1696 | 1697 | 1698 |
| v4chr3-37011m26 | 42 | 1699 | 1700 | 1701 |
| v4chr1-18457m31 | 42 | 1702 | 1703 | 1704 |
| v4chr1-19055m12 | 42 | 1705 | 1706 | 1707 |
| v4chr1-29009p15 | 42 | 1708 | 1709 | 1710 |
| v4chr1-38449p14 | 42 | 1711 | 1712 | 1713 |
| v4chr1-46331p6 | 42 | 1714 | 1715 | 1716 |
| v4chr1-59594m13 | 42 | 1717 | 1718 | 1719 |
| v4chr1-658m16 | 42 | 1720 | 1721 | 1722 |
| v4chr2-21734m13 | 42 | 1723 | 1724 | 1725 |
| v4chr2-2258m25 | 42 | 1726 | 1727 | 1728 |
| v4chr2-24773p8 | 42 | 1729 | 1730 | 1731 |
| v4chr2-6771p8 | 42 | 1732 | 1733 | 1734 |
| v4chr3-17003m16 | 42 | 1735 | 1736 | 1737 |
| v4chr3-18562m12 | 42 | 1738 | 1739 | 1740 |
| v4chr3-4805m13 | 42 | 1741 | 1742 | 1743 |
| v4chr3-9869m27 | 42 | 1744 | 1745 | 1746 |
| v4chr4-43989m8 | 42 | 1747 | 1748 | 1749 |
| v4chr4-45276p10 | 42 | 1750 | 1751 | 1752 |
| v4chr5-14800p13 | 42 | 1753 | 1754 | 1755 |
| v4chr5-24714m23 | 42 | 1756 | 1757 | 1758 |
| v4chr5-25789m17 | 42 | 1759 | 1760 | 1761 |
| v4chr5-37073m29 | 42 | 1762 | 1763 | 1764 |
| v4chr5-4568p8 | 42 | 1765 | 1766 | 1767 |
| v4chr5-4725p14 | 42 | 1768 | 1769 | 1770 |
| v4chr6a-10825m34 | 42 | 1771 | 1772 | 1773 |
| v4chr6a-11286m11 | 42 | 1774 | 1775 | 1776 |
| v4chr6a-12808m6 | 42 | 1777 | 1778 | 1779 |
| v4chr6a-29823m11 | 42 | 1780 | 1781 | 1782 |
| v4chr6b-14053m7 | 42 | 1783 | 1784 | 1785 |
| v4chr6b-5006p14 | 42 | 1786 | 1787 | 1788 |
| v4chr7-10105m16 | 42 | 1789 | 1790 | 1791 |
| v4chr7-17391p20 | 42 | 1792 | 1793 | 1794 |
| v4chr7-31980m8 | 42 | 1795 | 1796 | 1797 |
| v4chr7-8623p9 | 42 | 1798 | 1799 | 1800 |
| v4chr1-41835p9 | 42 | 1801 | 1802 | 1803 |
| v4chr2-50235m7 | 42 | 1804 | 1805 | 1806 |
| v4chr3-1588p16 | 42 | 1807 | 1808 | 1809 |
| v4chr3-37956p10 | 42 | 1810 | 1811 | 1812 |
| v4chr3-40728p9 | 42 | 1813 | 1814 | 1815 |
| v4chr4-39930p22 | 42 | 1816 | 1817 | 1818 |
| v4chr4-14266m25 | 42 | 1819 | 1820 | 1821 |
| v4chr1-316p17 | 42 | 1822 | 1823 | 1824 |
| v4chr2-37590m16 | 42 | 1825 | 1826 | 1827 |
| v4chr2-38027p10 | 42 | 1828 | 1829 | 1830 |
| v4chr2-51472p8 | 42 | 1831 | 1832 | 1833 |
| v4chr3-37035m11 | 42 | 1834 | 1835 | 1836 |
| v4chr4-26738p26 | 42 | 1837 | 1838 | 1839 |
| v4chr5-12327m19 | 42 | 1840 | 1841 | 1842 |
| v4chr5-1726m17 | 42 | 1843 | 1844 | 1845 |
| v4chr6a-1787p8 | 42 | 1846 | 1847 | 1848 |
| v4chr6a-24618m20 | 42 | 1849 | 1850 | 1851 |
| v4chr1-12499p14 | 42 | 1852 | 1853 | 1854 |
| v4chr1-28087p7 | 42 | 1855 | 1856 | 1857 |
| v4chr3-27671p10 | 42 | 1858 | 1859 | 1860 |
| v4chr4-39759p34 | 42 | 1861 | 1862 | 1863 |
| v4chr2-41104m9 | 42 | 1864 | 1865 | 1866 |
| v4chr7-23932p10 | 42 | 1867 | 1868 | 1869 |
| v4chr2-11600m10 | 42 | 1870 | 1871 | 1872 |
| v4chr2-32506m16 | 42 | 1873 | 1874 | 1875 |
| v4chr2-3338m10 | 42 | 1876 | 1877 | 1878 |
| v4chr2-34179m10 | 42 | 1879 | 1880 | 1881 |
| v4chr2-49538m26 | 42 | 1882 | 1883 | 1884 |
| v4chr6a-2135m6 | 42 | 1885 | 1886 | 1887 |
| v4chr1-35010m18 | 42 | 1888 | 1889 | 1890 |
| v4chr2-22667m34 | 42 | 1891 | 1892 | 1893 |
| v4chr2-60923m13 | 42 | 1894 | 1895 | 1896 |
| v4chr2-73549m17 | 42 | 1897 | 1898 | 1899 |
| v4chr3-21803m18 | 42 | 1900 | 1901 | 1902 |
| v4chr3-34414m31 | 42 | 1903 | 1904 | 1905 |
| v4chr3-45226p22 | 42 | 1906 | 1907 | 1908 |
| v4chr4-35696p12 | 42 | 1909 | 1910 | 1911 |
| v4chr5-1788m19 | 42 | 1912 | 1913 | 1914 |
| v4chr5-34086p20 | 42 | 1915 | 1916 | 1917 |
| v4chr5-35052p22 | 42 | 1918 | 1919 | 1920 |
| v4chr6a-12403p16 | 42 | 1921 | 1922 | 1923 |
| v4chr6a-20285m15 | 42 | 1924 | 1925 | 1926 |
| v4chr6a-2201p21 | 42 | 1927 | 1928 | 1929 |
| v4chr6a-33945p20 | 42 | 1930 | 1931 | 1932 |
| v4chr6b-2954m24 | 42 | 1933 | 1934 | 1935 |
| v4chr3-15966m8 | 42 | 1936 | 1937 | 1938 |
| v4chr4-49610p4 | 42 | 1939 | 1940 | 1941 |
| v4chr5-24567p17 | 42 | 1942 | 1943 | 1944 |
| v4chr2-22104m10 | 42 | 1945 | 1946 | 1947 |
| v4chr3-17131p5 | 42 | 1948 | 1949 | 1950 |
| v4chr2-65241p17 | 42 | 1951 | 1952 | 1953 |
| v4chr2-40183p30 | 42 | 1954 | 1955 | 1956 |
| v4chr1-58152p4 | 42 | 1957 | 1958 | 1959 |
| v4chr4-4363p19 | 42 | 1960 | 1961 | 1962 |
| v4chr4-18447p12 | 42 | 1963 | 1964 | 1965 |
| v4chr6b-4909m2 | 42 | 1966 | 1967 | 1968 |
| v4chr4-24094m10 | 42 | 1969 | 1970 | 1971 |
| v4chr2-34513p7 | 42 | 1972 | 1973 | 1974 |
| v4chr3-45077p8 | 42 | 1975 | 1976 | 1977 |
| v4chr6b-13786m3 | 42 | 1978 | 1979 | 1980 |
| v4chr5-1870m5 | 42 | 1981 | 1982 | 1983 |
| v4chr2-3364p7 | 42 | 1984 | 1985 | 1986 |
| v4chr6a-29671p4 | 42 | 1987 | 1988 | 1989 |
| v4chr4-5419m8 | 42 | 1990 | 1991 | 1992 |
| v4chr1-32074p19 | 42 | 1993 | 1994 | 1995 |
| v4s91-10m9 | 42 | 1996 | 1997 | 1998 |
| v4chr1-58177m15 | 42 | 1999 | 2000 | 2001 |
| v4chr2-54902m2 | 42 | 2002 | 2003 | 2004 |
| v4chr4-40293m7 | 42 | 2005 | 2006 | 2007 |
| v4chr5-1482m29 | 42 | 2008 | 2009 | 2010 |
| v4chr1-34411m30 | 42 | 2011 | 2012 | 2013 |
| v4chr5-7933m6 | 42 | 2014 | 2015 | 2016 |
| v4chr3-53351m4 | 42 | 2017 | 2018 | 2019 |
| v4chr3-4513p16 | 42 | 2020 | 2021 | 2022 |
| v4chr4-353m10 | 42 | 2023 | 2024 | 2025 |
| v4chr2-23470m5 | 42 | 2026 | 2027 | 2028 |
| v4chr7-36264p4 | 42 | 2029 | 2030 | 2031 |
| v4chr6b-13344p3 | 42 | 2032 | 2033 | 2034 |
| v4chr6b-4826p8 | 42 | 2035 | 2036 | 2037 |
| v4chr4-45532p6 | 42 | 2038 | 2039 | 2040 |
| v4chr5-47920p8 | 42 | 2041 | 2042 | 2043 |
| v4chr4-30032p5 | 42 | 2044 | 2045 | 2046 |
| v4chr2-73825m7 | 42 | 2047 | 2048 | 2049 |
| v4chr7-25060p34 | 42 | 2050 | 2051 | 2052 |
| v4chr2-14765p12 | 42 | 2053 | 2054 | 2055 |
| v4chr5-44106m10 | 42 | 2056 | 2057 | 2058 |
| v4chr4-5077m5 | 42 | 2059 | 2060 | 2061 |
| v4chr7-15349p3 | 42 | 2062 | 2063 | 2064 |
| v4chr3-45365m8 | 42 | 2065 | 2066 | 2067 |
| v4chr3-53853m17 | 42 | 2068 | 2069 | 2070 |
| v4chr6b-4805m7 | 42 | 2071 | 2072 | 2073 |
| v4chr7-9542m3 | 42 | 2074 | 2075 | 2076 |
| v4chr6a-29899m2 | 42 | 2077 | 2078 | 2079 |
| v4chr4-37575m28 | 43 | 2080 | 2081 | 2082 |
| v4chr6a-35773p8 | 10, 39 | 2083 | 2084 | 2085 |
| v4chr7-40326p7 | 10, 39 | 2086 | 2087 | 2088 |
| v4chr2-25453m14 | 12, 13 | 2089 | 2090 | 2091 |
| v4chr5-8405p13 | 12, 13 | 2092 | 2093 | 2094 |
| v4chr6a-36882m13 | 12, 13 | 2095 | 2096 | 2097 |
| v4chr4-5123p8 | 12, 13 | 2098 | 2099 | 2100 |
| v4chr6b-2202p8 | 12, 13 | 2101 | 2102 | 2103 |
| v4chr5-21401p27 | 17, 23, 31 | 2104 | 2105 | 2106 |
| v4chr1-48926p16 | 23, 29 | 2107 | 2108 | 2109 |
| v4chr5-19860m8 | 23, 29 | 2110 | 2111 | 2112 |
| v4chr5-42253p14 | 23, 29 | 2113 | 2114 | 2115 |
| v4chr7-40216p17 | 23, 29 | 2116 | 2117 | 2118 |
| v4chr3-2751m10 | 25, 26, 77 | 2119 | 2120 | 2121 |
| v4chr6a-36971m11 | 25, 40 | 2122 | 2123 | 2124 |
| v4chr4-544p8 | 25, 40 | 2125 | 2126 | 2127 |
| v4chr1-44026m16 | 3, 4, 7, 9 | 2128 | 2129 | 2130 |
| v4chr3-17994m25 | 3, 4, 7, 9 | 2131 | 2132 | 2133 |
| v4chr4-45310p16 | 3, 4, 7, 9 | 2134 | 2135 | 2136 |
| v4chr7-20937m20 | 3, 4, 7, 9 | 2137 | 2138 | 2139 |
| v4chr1-2290m26 | 3, 4, 7, 9, 16 | 2140 | 2141 | 2142 |
| v4chr3-8872m30 | 3, 4, 7, 9, 16 | 2143 | 2144 | 2145 |
| v4chr4-10676m29 | 3, 4, 7, 9, 16 | 2146 | 2147 | 2148 |
| v4chr4-8740m13 | 31, 42 | 2149 | 2150 | 2151 |
| v4chr6b-11432p12 | 34, 41 | 2152 | 2153 | 2154 |
| v4chr6a-31204m11 | 36, 37 | 2155 | 2156 | 2157 |
| v4chr3-36472m35 | 36, 37 | 2158 | 2159 | 2160 |
| v4chr5-26825p11 | 36, 37 | 2161 | 2162 | 2163 |
| v4chr2-11297m20 | 36, 38 | 2164 | 2165 | 2166 |
| v4chr2-67877p22 | 36, 38 | 2167 | 2168 | 2169 |
| v4chr2-39929m14 | 36, 38 | 2170 | 2171 | 2172 |
| v4chr5-4684m17 | 36, 39 | 2173 | 2174 | 2175 |
| v4chr3-17919m17 | 36, 39 | 2176 | 2177 | 2178 |
| v4chr2-30255p14 | 38, 39 | 2179 | 2180 | 2181 |
| v4chr1-58832m5 | 38, 39 | 2182 | 2183 | 2184 |
| v4chr2-32254p17 | 38, 39 | 2185 | 2186 | 2187 |
| v4chr3-13642p19 | 38, 39 | 2188 | 2189 | 2190 |
| v4chr1-59542m12 | 38, 39 | 2191 | 2192 | 2193 |
| v4chr5-1635p17 | 38, 39 | 2194 | 2195 | 2196 |
| v4chr6b-11019m11 | 38, 39 | 2197 | 2198 | 2199 |
| v4chr3-43052m16 | 5, 12, 13, 17, 23, 29 | 2200 | 2201 | 2202 |
| v4chr2-4364m12 | 5, 12, 13, 17, 23, 29 | 2203 | 2204 | 2205 |
| v4chr2-28581p16 | 5, 12, 13, 17, 23, 29, 31 | 2206 | 2207 | 2208 |
| v4chr5-1843m14 | 5, 12, 13, 17, 23, 29, 31 | 2209 | 2210 | 2211 |
| v4chr4-40955p12 | 5, 12, 13, 17, 23, 29, 39 | 2212 | 2213 | 2214 |
| v4chr2-14989m14 | 5, 17, 23 | 2215 | 2216 | 2217 |
| v4chr4-46773p15 | 5, 17, 23 | 2218 | 2219 | 2220 |
| v4chr4-11731p6 | 5, 17, 23 | 2221 | 2222 | 2223 |
| v4chr1-30263p17 | 7, 16, 23 | 2224 | 2225 | 2226 |
| v4chr6a-31316m27 | 8, 33 | 2227 | 2228 | 2229 |

TABLE 4

| | | Column 3 | Column 4 |
|---|---|---|---|
| Column 1 | Column 2 | SEQ ID NO | SEQ ID NO |
| V4 Gene Name | Activity # | (cDNA) | (protein) |
| v4chr2-57967p13 | 8 | 2230 | 2231 |
| v4chr4-30368p14 | 10 | 2232 | 2233 |
| v4chr6a-34248p21 | 10 | 2234 | 2235 |
| v4chr6a-6531m12 | 10 | 2236 | 2237 |
| v4chr3-35489p10 | 10 | 2238 | 2239 |
| v4chr2-75315p28 | 18 | 2240 | 2241 |
| v4chr5-1284p14 | 18 | 2242 | 2243 |
| v4chr5-35150p16 | 23 | 2244 | 2245 |
| v4chr4-10792p9 | 31 | 2246 | 2247 |
| v4chr5-22247p11 | 31 | 2248 | 2249 |
| v4chr4-8381m11 | 32 | 2250 | 2251 |
| v4chr2-102m8 | 32 | 2252 | 2253 |
| v4chr3-29604p12 | 32 | 2254 | 2255 |
| v4chr3-29950p24 | 32 | 2256 | 2257 |
| v4chr5-33742m9 | 32 | 2258 | 2259 |
| v4chr1-57128m5 | 35 | 2260 | 2261 |
| v4chr2-29475m5 | 35 | 2262 | 2263 |
| v4chr6a-21699m3 | 35 | 2264 | 2265 |
| v4chr6a-7011p4 | 35 | 2266 | 2267 |
| v4chr1-10342m10 | 35 | 2268 | 2269 |
| v4chr1-24352m9 | 35 | 2270 | 2271 |
| v4chr2-35788p7 | 35 | 2272 | 2273 |
| v4chr2-49232p1 | 35 | 2274 | 2275 |
| v4chr2-56739m14 | 35 | 2276 | 2277 |
| v4chr2-6238p30 | 35 | 2278 | 2279 |
| v4chr2-67374p5 | 35 | 2280 | 2281 |
| v4chr2-67381p5 | 35 | 2282 | 2283 |
| v4chr3-10827p39 | 35 | 2284 | 2285 |
| v4chr3-20418p15 | 35 | 2286 | 2287 |
| v4chr3-32214p23 | 35 | 2288 | 2289 |
| v4chr3-5272m30 | 35 | 2290 | 2291 |
| v4chr4-13901m27 | 35 | 2292 | 2293 |
| v4chr5-28107m2 | 35 | 2294 | 2295 |
| v4chr5-30039m3 | 35 | 2296 | 2297 |
| v4chr5-47293p25 | 35 | 2298 | 2299 |
| v4chr6a-20392p1 | 35 | 2300 | 2301 |
| v4chr6a-26707p16 | 35 | 2302 | 2303 |
| v4chr6a-28312m10 | 35 | 2304 | 2305 |
| v4chr6b-13516p5 | 35 | 2306 | 2307 |
| v4chr6b-6295m17 | 35 | 2308 | 2309 |
| v4chr2-51928m3 | 35 | 2310 | 2311 |
| v4chr6a-9639m4 | 35 | 2312 | 2313 |
| v4chr2-59513m23 | 35 | 2314 | 2315 |
| v4chr1-44319m12 | 35 | 2316 | 2317 |
| v4chr7-29969m11 | 15 | 2318 | 2319 |
| v4chr7-5968m3 | 35 | 2320 | 2321 |
| v4chr1-1016p9 | 36 | 2322 | 2323 |
| v4chr1-42021p10 | 36 | 2324 | 2325 |
| v4chr2-16764m2 | 36 | 2326 | 2327 |
| v4chr5-6468p14 | 36 | 2328 | 2329 |
| v4chr6b-11006m26 | 36 | 2330 | 2331 |
| v4chr6a-36811m20 | 36 | 2332 | 2333 |
| v4chr7-7858p22 | 36 | 2334 | 2335 |
| v4chr3-27759p48 | 36 | 2336 | 2337 |
| v4chr1-20263p19 | 36 | 2338 | 2339 |
| v4chr3-15735p1 | 36 | 2340 | 2341 |
| v4chr7-22160p18 | 36 | 2342 | 2343 |
| v4chr1-58242p77 | 38 | 2344 | 2345 |
| v4chr1-11216p13 | 38 | 2346 | 2347 |
| v4chr2-13006m13 | 38 | 2348 | 2349 |
| v4chr2-13195p9 | 38 | 2350 | 2351 |
| v4chr2-15305m9 | 38 | 2352 | 2353 |
| v4chr2-65759m8 | 38 | 2354 | 2355 |
| v4chr2-7538p12 | 38 | 2356 | 2357 |
| v4chr3-14746m19 | 38 | 2358 | 2359 |
| v4chr3-34344m14 | 38 | 2360 | 2361 |
| v4chr3-38833m11 | 38 | 2362 | 2363 |
| v4chr4-11720m13 | 38 | 2364 | 2365 |
| v4chr4-233p34 | 38 | 2366 | 2367 |
| v4chr5-15654p12 | 38 | 2368 | 2369 |
| v4chr5-22765m17 | 38 | 2370 | 2371 |
| v4chr5-48100m10 | 38 | 2372 | 2373 |
| v4chr5-48229p43 | 38 | 2374 | 2375 |
| v4chr5-7373p14 | 38 | 2376 | 2377 |
| v4chr5-7536p13 | 38 | 2378 | 2379 |
| v4chr6a-24583m8 | 38 | 2380 | 2381 |
| v4chr6a-36579p14 | 38 | 2382 | 2383 |
| v4s92-1p7 | 38 | 2384 | 2385 |
| v4chr1-61193p17 | 38 | 2386 | 2387 |
| v4chr2-43661p17 | 38 | 2388 | 2389 |
| v4chr5-27033p17 | 38 | 2390 | 2391 |
| v4chr3-48895p17 | 38 | 2392 | 2393 |
| v4chr5-27144m12 | 38 | 2394 | 2395 |
| v4chr6a-1903p12 | 38 | 2396 | 2397 |
| v4chr5-18969m2 | 38 | 2398 | 2399 |
| v4chr2-22980m18 | 38 | 2400 | 2401 |
| v4chr1-13935m8 | 38 | 2402 | 2403 |
| v4chr1-23745m10 | 38 | 2404 | 2405 |
| v4chr1-34341p25 | 38 | 2406 | 2407 |
| v4chr1-35963m9 | 38 | 2408 | 2409 |
| v4chr1-36783p3 | 38 | 2410 | 2411 |
| v4chr2-18405p8 | 38 | 2412 | 2413 |
| v4chr3-107p128 | 38 | 2414 | 2415 |
| v4chr3-13405p10 | 38 | 2416 | 2417 |
| v4chr3-2904p7 | 38 | 2418 | 2419 |
| v4chr3-2942m15 | 38 | 2420 | 2421 |
| v4chr3-33137m15 | 38 | 2422 | 2423 |
| v4chr3-43021p11 | 38 | 2424 | 2425 |
| v4chr3-47814m11 | 38 | 2426 | 2427 |
| v4chr5-10384p13 | 38 | 2428 | 2429 |
| v4chr5-37220p21 | 38 | 2430 | 2431 |
| v4chr6a-32269m13 | 38 | 2432 | 2433 |
| v4chr6a-33027m16 | 38 | 2434 | 2435 |
| v4chr6a-36237m12 | 38 | 2436 | 2437 |
| v4chr6a-36330p35 | 38 | 2438 | 2439 |
| v4chr7-12605p12 | 38 | 2440 | 2441 |
| v4chr7-17382m11 | 38 | 2442 | 2443 |
| v4chr7-17572p26 | 38 | 2444 | 2445 |
| v4chr5-42512m6 | 38 | 2446 | 2447 |
| v4chr2-64405m4 | 38 | 2448 | 2449 |
| v4chr3-33074m14 | 38 | 2450 | 2451 |
| v4chr2-6305p19 | 38 | 2452 | 2453 |
| v4chr2-64474m26 | 38 | 2454 | 2455 |
| v4chr5-42644p8 | 38 | 2456 | 2457 |
| v4chr7-27945m13 | 38 | 2458 | 2459 |
| v4chr1-42179p8 | 38 | 2460 | 2461 |
| v4chr4-16780m3 | 39 | 2462 | 2463 |
| v4chr4-1751m13 | 39 | 2464 | 2465 |
| v4chr6a-33270m2 | 39 | 2466 | 2467 |
| v4chr6a-35190m15 | 39 | 2468 | 2469 |
| v4chr1-30780p3 | 39 | 2470 | 2471 |
| v4chr6a-21075m13 | 39 | 2472 | 2473 |
| v4chr4-79p11 | 39 | 2474 | 2475 |
| v4chr3-4968p7 | 39 | 2476 | 2477 |
| v4chr1-19382p14 | 39 | 2478 | 2479 |
| v4chr1-31342m12 | 39 | 2480 | 2481 |
| v4chr2-39778m27 | 39 | 2482 | 2483 |
| v4chr2-57058m18 | 39 | 2484 | 2485 |
| v4chr3-22185m8 | 39 | 2486 | 2487 |
| v4chr3-23948p3 | 39 | 2488 | 2489 |
| v4chr3-24403p12 | 39 | 2490 | 2491 |
| v4chr4-30291p15 | 39 | 2492 | 2493 |
| v4chr5-37453p30 | 39 | 2494 | 2495 |
| v4chr6a-20109m18 | 39 | 2496 | 2497 |
| v4chr6a-31273m14 | 39 | 2498 | 2499 |
| v4chr7-24161m11 | 39 | 2500 | 2501 |
| v4chr7-28176p16 | 39 | 2502 | 2503 |
| v4chr2-60711m10 | 39 | 2504 | 2505 |
| v4chr6a-21577m11 | 39 | 2506 | 2507 |
| v4chr2-12898m12 | 39 | 2508 | 2509 |
| v4chr2-40989m18 | 39 | 2510 | 2511 |
| v4chr5-33986p11 | 39 | 2512 | 2513 |
| v4chr7-30073p18 | 39 | 2514 | 2515 |
| v4chr2-15289m19 | 39 | 2516 | 2517 |
| v4chr1-36927m18 | 42 | 2518 | 2519 |
| v4chr1-38528p24 | 42 | 2520 | 2521 |
| v4chr2-12190m22 | 42 | 2522 | 2523 |
| v4chr2-35123m22 | 42 | 2524 | 2525 |
| v4chr2-54492p19 | 42 | 2526 | 2527 |
| v4chr2-67346p20 | 42 | 2528 | 2529 |
| v4chr3-14610p39 | 42 | 2530 | 2531 |
| v4chr3-31901p20 | 42 | 2532 | 2533 |
| v4chr3-32857m23 | 42 | 2534 | 2535 |
| v4chr3-6011m14 | 42 | 2536 | 2537 |
| v4chr4-12617m50 | 42 | 2538 | 2539 |
| v4chr4-32764m22 | 42 | 2540 | 2541 |
| v4chr5-4658m11 | 42 | 2542 | 2543 |
| v4chr6a-20234p24 | 42 | 2544 | 2545 |
| v4chr6a-36249p67 | 42 | 2546 | 2547 |
| v4chr6b-15163p8 | 42 | 2548 | 2549 |
| v4chr7-10730m10 | 42 | 2550 | 2551 |
| v4chr7-35558p24 | 42 | 2552 | 2553 |
| v4chr7-671m18 | 42 | 2554 | 2555 |
| v4chr1-11153p5 | 42 | 2556 | 2557 |
| v4chr1-11835p4 | 42 | 2558 | 2559 |
| v4chr1-15949m10 | 42 | 2560 | 2561 |
| v4chr1-16699m32 | 42 | 2562 | 2563 |
| v4chr1-16918p5 | 42 | 2564 | 2565 |
| v4chr1-16961m17 | 42 | 2566 | 2567 |
| v4chr1-20746p2 | 42 | 2568 | 2569 |
| v4chr1-27385p6 | 42 | 2570 | 2571 |
| v4chr1-33548m7 | 42 | 2572 | 2573 |
| v4chr1-40336m16 | 42 | 2574 | 2575 |
| v4chr1-42493m15 | 42 | 2576 | 2577 |
| v4chr1-44691p22 | 42 | 2578 | 2579 |
| v4chr1-45822m5 | 42 | 2580 | 2581 |
| v4chr1-4789m6 | 42 | 2582 | 2583 |
| v4chr1-53321m12 | 42 | 2584 | 2585 |
| v4chr1-60843m17 | 42 | 2586 | 2587 |
| v4chr1-60918p8 | 42 | 2588 | 2589 |
| v4chr1-92p3 | 42 | 2590 | 2591 |
| v4chr2-13761m17 | 42 | 2592 | 2593 |
| v4chr2-19452m21 | 42 | 2594 | 2595 |
| v4chr2-27543p31 | 42 | 2596 | 2597 |
| v4chr2-30583p14 | 42 | 2598 | 2599 |
| v4chr2-32330m15 | 42 | 2600 | 2601 |
| v4chr2-38952m22 | 42 | 2602 | 2603 |
| v4chr2-4568m14 | 42 | 2604 | 2605 |
| v4chr2-52229m15 | 42 | 2606 | 2607 |
| v4chr2-53474p31 | 42 | 2608 | 2609 |
| v4chr2-53765m21 | 42 | 2610 | 2611 |
| v4chr2-54700m23 | 42 | 2612 | 2613 |
| v4chr2-55513m27 | 42 | 2614 | 2615 |
| v4chr2-5611p7 | 42 | 2616 | 2617 |
| v4chr2-56555m10 | 42 | 2618 | 2619 |
| v4chr2-57916m11 | 42 | 2620 | 2621 |
| v4chr2-62884p2 | 42 | 2622 | 2623 |
| v4chr2-7523p12 | 42 | 2624 | 2625 |
| v4chr2-76633p12 | 42 | 2626 | 2627 |
| v4chr3-10084p14 | 42 | 2628 | 2629 |
| v4chr3-11413m21 | 42 | 2630 | 2631 |
| v4chr3-18181p23 | 42 | 2632 | 2633 |
| v4chr3-19556p21 | 42 | 2634 | 2635 |
| v4chr3-19836p12 | 42 | 2636 | 2637 |
| v4chr3-29571p15 | 42 | 2638 | 2639 |
| v4chr3-29625m2 | 42 | 2640 | 2641 |
| v4chr3-33354m8 | 42 | 2642 | 2643 |
| v4chr3-46992m18 | 42 | 2644 | 2645 |
| v4chr3-53399m10 | 42 | 2646 | 2647 |
| v4chr3-54020m5 | 42 | 2648 | 2649 |
| v4chr4-10877p2 | 42 | 2650 | 2651 |
| v4chr4-10886p2 | 42 | 2652 | 2653 |
| v4chr4-23034m5 | 42 | 2654 | 2655 |
| v4chr4-29571m3 | 42 | 2656 | 2657 |
| v4chr4-31475p8 | 42 | 2658 | 2659 |
| v4chr4-33687p20 | 42 | 2660 | 2661 |
| v4chr4-34420p9 | 42 | 2662 | 2663 |
| v4chr4-35829p3 | 42 | 2664 | 2665 |
| v4chr4-37061p11 | 42 | 2666 | 2667 |
| v4chr4-38086m11 | 42 | 2668 | 2669 |
| v4chr4-38290p9 | 42 | 2670 | 2671 |
| v4chr4-39m3 | 42 | 2672 | 2673 |
| v4chr4-44118m12 | 42 | 2674 | 2675 |
| v4chr4-44126p15 | 42 | 2676 | 2677 |
| v4chr4-45943p16 | 42 | 2678 | 2679 |
| v4chr4-46301m15 | 42 | 2680 | 2681 |
| v4chr4-48m1 | 42 | 2682 | 2683 |
| v4chr4-4994m16 | 42 | 2684 | 2685 |
| v4chr4-5080p2 | 42 | 2686 | 2687 |
| v4chr4-59p12 | 42 | 2688 | 2689 |
| v4chr5-14015m13 | 42 | 2690 | 2691 |
| v4chr5-17807m9 | 42 | 2692 | 2693 |
| v4chr5-21212m9 | 42 | 2694 | 2695 |
| v4chr5-24104m9 | 42 | 2696 | 2697 |
| v4chr5-29215p9 | 42 | 2698 | 2699 |
| v4chr5-30797p15 | 42 | 2700 | 2701 |
| v4chr5-39582p2 | 42 | 2702 | 2703 |
| v4chr5-40543m23 | 42 | 2704 | 2705 |
| v4chr5-4055m3 | 42 | 2706 | 2707 |
| v4chr5-41892m21 | 42 | 2708 | 2709 |
| v4chr5-41950m21 | 42 | 2710 | 2711 |
| v4chr5-41987p18 | 42 | 2712 | 2713 |
| v4chr5-42324m15 | 42 | 2714 | 2715 |
| v4chr5-47079m20 | 42 | 2716 | 2717 |
| v4chr5-48077m19 | 42 | 2718 | 2719 |
| v4chr5-7142p15 | 42 | 2720 | 2721 |
| v4chr5-7401p13 | 42 | 2722 | 2723 |
| v4chr6a-1007m23 | 42 | 2724 | 2725 |
| v4chr6a-17837m9 | 42 | 2726 | 2727 |
| v4chr6a-18445m21 | 42 | 2728 | 2729 |
| v4chr6a-2028p20 | 42 | 2730 | 2731 |
| v4chr6a-24937p14 | 42 | 2732 | 2733 |
| v4chr6a-2523m28 | 42 | 2734 | 2735 |
| v4chr6a-31250p5 | 42 | 2736 | 2737 |
| v4chr6a-32548m21 | 42 | 2738 | 2739 |
| v4chr6a-8301m21 | 42 | 2740 | 2741 |
| v4chr6b-9990p9 | 42 | 2742 | 2743 |
| v4chr7-16646p21 | 42 | 2744 | 2745 |
| v4chr7-16874m23 | 42 | 2746 | 2747 |
| v4chr7-19621m3 | 42 | 2748 | 2749 |
| v4chr7-22059m12 | 42 | 2750 | 2751 |
| v4chr7-23684m5 | 42 | 2752 | 2753 |
| v4chr7-27097p4 | 42 | 2754 | 2755 |
| v4chr7-4422m13 | 42 | 2756 | 2757 |
| v4chr7-5069p4 | 42 | 2758 | 2759 |
| v4chr7-5943p2 | 42 | 2760 | 2761 |
| v4chr7-7783p6 | 42 | 2762 | 2763 |
| v4chr7-9400p7 | 42 | 2764 | 2765 |
| v4chr7-9639p29 | 42 | 2766 | 2767 |
| v4s93-8m8 | 42 | 2768 | 2769 |
| v4chr6b-11029m4 | 42 | 2770 | 2771 |
| v4chr1-1342p12 | 42 | 2772 | 2773 |
| v4chr1-12623p16 | 42 | 2774 | 2775 |
| v4chr1-1599p15 | 42 | 2776 | 2777 |
| v4chr1-31206p15 | 42 | 2778 | 2779 |
| v4chr1-35179m35 | 42 | 2780 | 2781 |
| v4chr1-48321p14 | 42 | 2782 | 2783 |
| v4chr2-1421p3 | 42 | 2784 | 2785 |
| v4chr2-37074m14 | 42 | 2786 | 2787 |
| v4chr2-39539m6 | 42 | 2788 | 2789 |
| v4chr2-5362m7 | 42 | 2790 | 2791 |
| v4chr2-5452m11 | 42 | 2792 | 2793 |
| v4chr3-13375p20 | 42 | 2794 | 2795 |
| v4chr3-16302p12 | 42 | 2796 | 2797 |
| v4chr3-17575m14 | 42 | 2798 | 2799 |
| v4chr3-30304m10 | 42 | 2800 | 2801 |
| v4chr3-47790p11 | 42 | 2802 | 2803 |
| v4chr4-1372m16 | 42 | 2804 | 2805 |
| v4chr4-24579m13 | 42 | 2806 | 2807 |
| v4chr4-29387m10 | 42 | 2808 | 2809 |
| v4chr4-42943p10 | 42 | 2810 | 2811 |
| v4chr4-45508m10 | 42 | 2812 | 2813 |
| v4chr4-47359m11 | 42 | 2814 | 2815 |
| v4chr4-5521m10 | 42 | 2816 | 2817 |
| v4chr5-27169p7 | 42 | 2818 | 2819 |
| v4chr5-42553p23 | 42 | 2820 | 2821 |
| v4chr5-47036m14 | 42 | 2822 | 2823 |
| v4chr6a-15505p16 | 42 | 2824 | 2825 |
| v4chr6a-21039p16 | 42 | 2826 | 2827 |
| v4chr6a-25179m21 | 42 | 2828 | 2829 |
| v4chr6a-25554p8 | 42 | 2830 | 2831 |
| v4chr6a-2702p25 | 42 | 2832 | 2833 |
| v4chr6a-29857m20 | 42 | 2834 | 2835 |
| v4chr6a-31214m12 | 42 | 2836 | 2837 |
| v4chr6a-32888m5 | 42 | 2838 | 2839 |
| v4chr6a-4208p19 | 42 | 2840 | 2841 |
| v4chr6b-15657p13 | 42 | 2842 | 2843 |
| v4chr7-29800m7 | 42 | 2844 | 2845 |
| v4chr7-40370m16 | 42 | 2846 | 2847 |
| v4chr2-7221m34 | 42 | 2848 | 2849 |
| v4chr3-8110m9 | 42 | 2850 | 2851 |
| v4chr6a-33136p14 | 42 | 2852 | 2853 |
| v4chr1-7227m3 | 42 | 2854 | 2855 |
| v4chr1-41494m68 | 42 | 2856 | 2857 |
| v4chr2-38415p3 | 42 | 2858 | 2859 |
| v4chr3-17199p22 | 42 | 2860 | 2861 |
| v4chr4-20855p3 | 42 | 2862 | 2863 |
| v4chr4-49649p17 | 42 | 2864 | 2865 |
| v4chr6a-24964p4 | 42 | 2866 | 2867 |
| v4chr7-16614p25 | 42 | 2868 | 2869 |
| v4chr7-20943p7 | 42 | 2870 | 2871 |
| v4chr1-14975m4 | 42 | 2872 | 2873 |
| v4chr1-18229m10 | 42 | 2874 | 2875 |
| v4chr1-29730m21 | 42 | 2876 | 2877 |
| v4chr1-30186p3 | 42 | 2878 | 2879 |
| v4chr1-39789p11 | 42 | 2880 | 2881 |
| v4chr1-46006p10 | 42 | 2882 | 2883 |
| v4chr1-58226m21 | 42 | 2884 | 2885 |
| v4chr1-59485m17 | 42 | 2886 | 2887 |
| v4chr1-7573m14 | 42 | 2888 | 2889 |
| v4chr1-9460p19 | 42 | 2890 | 2891 |
| v4chr2-25244p14 | 42 | 2892 | 2893 |
| v4chr2-37328p15 | 42 | 2894 | 2895 |
| v4chr2-43062p8 | 42 | 2896 | 2897 |
| v4chr2-44880p5 | 42 | 2898 | 2899 |
| v4chr2-52279p2 | 42 | 2900 | 2901 |
| v4chr2-56459m8 | 42 | 2902 | 2903 |
| v4chr2-67387p8 | 42 | 2904 | 2905 |
| v4chr3-37717m2 | 42 | 2906 | 2907 |
| v4chr3-37835p11 | 42 | 2908 | 2909 |
| v4chr3-41013m7 | 42 | 2910 | 2911 |
| v4chr3-49242m70 | 42 | 2912 | 2913 |
| v4chr3-54209m3 | 42 | 2914 | 2915 |
| v4chr4-11673p26 | 42 | 2916 | 2917 |
| v4chr4-32181p22 | 42 | 2918 | 2919 |
| v4chr4-32209p12 | 42 | 2920 | 2921 |
| v4chr4-37747m6 | 42 | 2922 | 2923 |
| v4chr4-46893m12 | 42 | 2924 | 2925 |
| v4chr4-741p25 | 42 | 2926 | 2927 |
| v4chr5-1574m4 | 42 | 2928 | 2929 |
| v4chr5-21955m8 | 42 | 2930 | 2931 |
| v4chr5-33888p11 | 42 | 2932 | 2933 |
| v4chr5-43589p8 | 42 | 2934 | 2935 |
| v4chr5-5270p15 | 42 | 2936 | 2937 |
| v4chr6a-2445m74 | 42 | 2938 | 2939 |
| v4chr6a-33493p4 | 42 | 2940 | 2941 |
| v4chr6a-35131p8 | 42 | 2942 | 2943 |
| v4chr6b-13007p73 | 42 | 2944 | 2945 |
| v4chr6b-13196p16 | 42 | 2946 | 2947 |
| v4chr6b-5062p9 | 42 | 2948 | 2949 |
| v4chr6b-6804m19 | 42 | 2950 | 2951 |
| v4chr7-33594p4 | 42 | 2952 | 2953 |
| v4chr7-9714m9 | 42 | 2954 | 2955 |
| v4chr1-28039m8 | 42 | 2956 | 2957 |
| v4chr2-42568p10 | 42 | 2958 | 2959 |
| v4chr3-31895m18 | 42 | 2960 | 2961 |
| v4chr5-20061p16 | 42 | 2962 | 2963 |
| v4chr5-41565p18 | 42 | 2964 | 2965 |
| v4chr5-48181m13 | 42 | 2966 | 2967 |
| v4chr5-6553p15 | 42 | 2968 | 2969 |
| v4chr7-7172m8 | 42 | 2970 | 2971 |
| v4s130-0p13 | 42 | 2972 | 2973 |
| v4chr2-28034p12 | 42 | 2974 | 2975 |
| v4chr1-2319p6 | 42 | 2976 | 2977 |
| v4chr1-32261m25 | 42 | 2978 | 2979 |
| v4chr1-34595p2 | 42 | 2980 | 2981 |
| v4chr2-44877m11 | 42 | 2982 | 2983 |
| v4chr2-62070m10 | 42 | 2984 | 2985 |
| v4chr3-16763m9 | 42 | 2986 | 2987 |
| v4chr3-21425m10 | 42 | 2988 | 2989 |
| v4chr4-37949p14 | 42 | 2990 | 2991 |
| v4chr5-36448m5 | 42 | 2992 | 2993 |
| v4chr6a-17641m6 | 42 | 2994 | 2995 |
| v4chr6b-15968m6 | 42 | 2996 | 2997 |
| v4chr6a-21380m48 | 42 | 2998 | 2999 |
| v4chr1-10457p32 | 42 | 3000 | 3001 |
| v4chr1-10807p3 | 42 | 3002 | 3003 |
| v4chr1-10977m4 | 42 | 3004 | 3005 |
| v4chr1-1137m14 | 42 | 3006 | 3007 |
| v4chr1-115p76 | 42 | 3008 | 3009 |
| v4chr1-13178p15 | 42 | 3010 | 3011 |
| v4chr1-13540m57 | 42 | 3012 | 3013 |
| v4chr1-13716m21 | 42 | 3014 | 3015 |
| v4chr1-1420p3 | 42 | 3016 | 3017 |
| v4chr1-14403p4 | 42 | 3018 | 3019 |
| v4chr1-14704m4 | 42 | 3020 | 3021 |
| v4chr1-15025p41 | 42 | 3022 | 3023 |
| v4chr1-16031p4 | 42 | 3024 | 3025 |
| v4chr1-16390p8 | 42 | 3026 | 3027 |
| v4chr1-16579p17 | 42 | 3028 | 3029 |
| v4chr1-1803p6 | 42 | 3030 | 3031 |
| v4chr1-18071m20 | 42 | 3032 | 3033 |
| v4chr1-18485p50 | 42 | 3034 | 3035 |
| v4chr1-18818m14 | 42 | 3036 | 3037 |
| v4chr1-19076m8 | 42 | 3038 | 3039 |
| v4chr1-20140m6 | 42 | 3040 | 3041 |
| v4chr1-20154m6 | 42 | 3042 | 3043 |
| v4chr1-21117p25 | 42 | 3044 | 3045 |
| v4chr1-21183p4 | 42 | 3046 | 3047 |
| v4chr1-21466p8 | 42 | 3048 | 3049 |
| v4chr1-21521m8 | 42 | 3050 | 3051 |
| v4chr1-21704m13 | 42 | 3052 | 3053 |
| v4chr1-21714p16 | 42 | 3054 | 3055 |
| v4chr1-22304m3 | 42 | 3056 | 3057 |
| v4chr1-23950p61 | 42 | 3058 | 3059 |
| v4chr1-24020p6 | 42 | 3060 | 3061 |
| v4chr1-24145p10 | 42 | 3062 | 3063 |
| v4chr1-24984m46 | 42 | 3064 | 3065 |
| v4chr1-25609p4 | 42 | 3066 | 3067 |
| v4chr1-26155p15 | 42 | 3068 | 3069 |
| v4chr1-27175p10 | 42 | 3070 | 3071 |
| v4chr1-27186p4 | 42 | 3072 | 3073 |
| v4chr1-28117m15 | 42 | 3074 | 3075 |
| v4chr1-28331m9 | 42 | 3076 | 3077 |
| v4chr1-28483m9 | 42 | 3078 | 3079 |
| v4chr1-28516m24 | 42 | 3080 | 3081 |
| v4chr1-28558m23 | 42 | 3082 | 3083 |
| v4chr1-29139m20 | 42 | 3084 | 3085 |
| v4chr1-29250p11 | 42 | 3086 | 3087 |
| v4chr1-29352p10 | 42 | 3088 | 3089 |
| v4chr1-30081m6 | 42 | 3090 | 3091 |
| v4chr1-30606m7 | 42 | 3092 | 3093 |
| v4chr1-30977p16 | 42 | 3094 | 3095 |
| v4chr1-32218p13 | 42 | 3096 | 3097 |
| v4chr1-32271p6 | 42 | 3098 | 3099 |
| v4chr1-33102p20 | 42 | 3100 | 3101 |
| v4chr1-33204p35 | 42 | 3102 | 3103 |
| v4chr1-33938m12 | 42 | 3104 | 3105 |
| v4chr1-34083m18 | 42 | 3106 | 3107 |
| v4chr1-34251m14 | 42 | 3108 | 3109 |
| v4chr1-34272m9 | 42 | 3110 | 3111 |
| v4chr1-34290m13 | 42 | 3112 | 3113 |
| v4chr1-34770m14 | 42 | 3114 | 3115 |
| v4chr1-34778p6 | 42 | 3116 | 3117 |
| v4chr1-35116p15 | 42 | 3118 | 3119 |
| v4chr1-35357m18 | 42 | 3120 | 3121 |
| v4chr1-35632m24 | 42 | 3122 | 3123 |
| v4chr1-35735m12 | 42 | 3124 | 3125 |
| v4chr1-36217m19 | 42 | 3126 | 3127 |
| v4chr1-36240p9 | 42 | 3128 | 3129 |
| v4chr1-36262p21 | 42 | 3130 | 3131 |
| v4chr1-36396m30 | 42 | 3132 | 3133 |
| v4chr1-36907m7 | 42 | 3134 | 3135 |
| v4chr1-37291p21 | 42 | 3136 | 3137 |
| v4chr1-38271m59 | 42 | 3138 | 3139 |
| v4chr1-38352m10 | 42 | 3140 | 3141 |
| v4chr1-38599p34 | 42 | 3142 | 3143 |
| v4chr1-38787m13 | 42 | 3144 | 3145 |
| v4chr1-39728p10 | 42 | 3146 | 3147 |
| v4chr1-40466m27 | 42 | 3148 | 3149 |
| v4chr1-40917p10 | 42 | 3150 | 3151 |
| v4chr1-4155p78 | 42 | 3152 | 3153 |
| v4chr1-41796m7 | 42 | 3154 | 3155 |
| v4chr1-42151m23 | 42 | 3156 | 3157 |
| v4chr1-43155m34 | 42 | 3158 | 3159 |
| v4chr1-43278p27 | 42 | 3160 | 3161 |
| v4chr1-43613p19 | 42 | 3162 | 3163 |
| v4chr1-43673p9 | 42 | 3164 | 3165 |
| v4chr1-43834p9 | 42 | 3166 | 3167 |
| v4chr1-44342m4 | 42 | 3168 | 3169 |
| v4chr1-44665m4 | 42 | 3170 | 3171 |
| v4chr1-45191p15 | 42 | 3172 | 3173 |
| v4chr1-45572p12 | 42 | 3174 | 3175 |
| v4chr1-46225p28 | 42 | 3176 | 3177 |
| v4chr1-46506m12 | 42 | 3178 | 3179 |
| v4chr1-46699p32 | 42 | 3180 | 3181 |
| v4chr1-46982p6 | 42 | 3182 | 3183 |
| v4chr1-47860m36 | 42 | 3184 | 3185 |
| v4chr1-47879p5 | 42 | 3186 | 3187 |
| v4chr1-48580m1 | 42 | 3188 | 3189 |
| v4chr1-51061p10 | 42 | 3190 | 3191 |
| v4chr1-51207m10 | 42 | 3192 | 3193 |
| v4chr1-51607m16 | 42 | 3194 | 3195 |
| v4chr1-52501m14 | 42 | 3196 | 3197 |
| v4chr1-5287m11 | 42 | 3198 | 3199 |
| v4chr1-54372m19 | 42 | 3200 | 3201 |
| v4chr1-55352p18 | 42 | 3202 | 3203 |
| v4chr1-55379m4 | 42 | 3204 | 3205 |
| v4chr1-57857p4 | 42 | 3206 | 3207 |
| v4chr1-58098p9 | 42 | 3208 | 3209 |
| v4chr1-58411p9 | 42 | 3210 | 3211 |
| v4chr1-60322m1 | 42 | 3212 | 3213 |
| v4chr1-60636p8 | 42 | 3214 | 3215 |
| v4chr1-60998p8 | 42 | 3216 | 3217 |
| v4chr1-61050m12 | 42 | 3218 | 3219 |
| v4chr1-6331p10 | 42 | 3220 | 3221 |
| v4chr1-6594m15 | 42 | 3222 | 3223 |
| v4chr1-8392p14 | 42 | 3224 | 3225 |
| v4chr2-1059m9 | 42 | 3226 | 3227 |
| v4chr2-11224p3 | 42 | 3228 | 3229 |
| v4chr2-11338p40 | 42 | 3230 | 3231 |
| v4chr2-11564m15 | 42 | 3232 | 3233 |
| v4chr2-11908m15 | 42 | 3234 | 3235 |
| v4chr2-12406m43 | 42 | 3236 | 3237 |
| v4chr2-12521p2 | 42 | 3238 | 3239 |
| v4chr2-13137m13 | 42 | 3240 | 3241 |
| v4chr2-13301m7 | 42 | 3242 | 3243 |
| v4chr2-13345p33 | 42 | 3244 | 3245 |
| v4chr2-13436m9 | 42 | 3246 | 3247 |
| v4chr2-13629m23 | 42 | 3248 | 3249 |
| v4chr2-14235m4 | 42 | 3250 | 3251 |
| v4chr2-14299p7 | 42 | 3252 | 3253 |
| v4chr2-14397m19 | 42 | 3254 | 3255 |
| v4chr2-14463m23 | 42 | 3256 | 3257 |
| v4chr2-14610p20 | 42 | 3258 | 3259 |
| v4chr2-14704p7 | 42 | 3260 | 3261 |
| v4chr2-14713p27 | 42 | 3262 | 3263 |
| v4chr2-14824p5 | 42 | 3264 | 3265 |
| v4chr2-16483p18 | 42 | 3266 | 3267 |
| v4chr2-16672p31 | 42 | 3268 | 3269 |
| v4chr2-16932p23 | 42 | 3270 | 3271 |
| v4chr2-17576m23 | 42 | 3272 | 3273 |
| v4chr2-17666m10 | 42 | 3274 | 3275 |
| v4chr2-18335p7 | 42 | 3276 | 3277 |
| v4chr2-18793p61 | 42 | 3278 | 3279 |
| v4chr2-19372m25 | 42 | 3280 | 3281 |
| v4chr2-19765p37 | 42 | 3282 | 3283 |
| v4chr2-2029m32 | 42 | 3284 | 3285 |
| v4chr2-20371p11 | 42 | 3286 | 3287 |
| v4chr2-20887m19 | 42 | 3288 | 3289 |
| v4chr2-20974m6 | 42 | 3290 | 3291 |
| v4chr2-2104p20 | 42 | 3292 | 3293 |
| v4chr2-21090m9 | 42 | 3294 | 3295 |
| v4chr2-22126m13 | 42 | 3296 | 3297 |
| v4chr2-22174m20 | 42 | 3298 | 3299 |
| v4chr2-22578m10 | 42 | 3300 | 3301 |
| v4chr2-23133m18 | 42 | 3302 | 3303 |
| v4chr2-23145p32 | 42 | 3304 | 3305 |
| v4chr2-23352m14 | 42 | 3306 | 3307 |
| v4chr2-23400m9 | 42 | 3308 | 3309 |
| v4chr2-23606p10 | 42 | 3310 | 3311 |
| v4chr2-23641m19 | 42 | 3312 | 3313 |
| v4chr2-24013m11 | 42 | 3314 | 3315 |
| v4chr2-24127p12 | 42 | 3316 | 3317 |
| v4chr2-24157m14 | 42 | 3318 | 3319 |
| v4chr2-24193m23 | 42 | 3320 | 3321 |
| v4chr2-24278m17 | 42 | 3322 | 3323 |
| v4chr2-24327m15 | 42 | 3324 | 3325 |
| v4chr2-24382p4 | 42 | 3326 | 3327 |
| v4chr2-25410p17 | 42 | 3328 | 3329 |
| v4chr2-25629p18 | 42 | 3330 | 3331 |
| v4chr2-25652p10 | 42 | 3332 | 3333 |
| v4chr2-27137p26 | 42 | 3334 | 3335 |
| v4chr2-27185m4 | 42 | 3336 | 3337 |
| v4chr2-27429m8 | 42 | 3338 | 3339 |
| v4chr2-28115m16 | 42 | 3340 | 3341 |
| v4chr2-28126p57 | 42 | 3342 | 3343 |
| v4chr2-28820m3 | 42 | 3344 | 3345 |
| v4chr2-29557p3 | 42 | 3346 | 3347 |
| v4chr2-30307m3 | 42 | 3348 | 3349 |
| v4chr2-310p13 | 42 | 3350 | 3351 |
| v4chr2-31207p16 | 42 | 3352 | 3353 |
| v4chr2-3121m10 | 42 | 3354 | 3355 |
| v4chr2-33023m2 | 42 | 3356 | 3357 |
| v4chr2-33036p10 | 42 | 3358 | 3359 |
| v4chr2-33227m16 | 42 | 3360 | 3361 |
| v4chr2-33234m3 | 42 | 3362 | 3363 |
| v4chr2-33286p24 | 42 | 3364 | 3365 |
| v4chr2-33439m16 | 42 | 3366 | 3367 |
| v4chr2-33754m11 | 42 | 3368 | 3369 |
| v4chr2-3400m23 | 42 | 3370 | 3371 |
| v4chr2-34222p22 | 42 | 3372 | 3373 |
| v4chr2-34288m13 | 42 | 3374 | 3375 |
| v4chr2-3447m15 | 42 | 3376 | 3377 |
| v4chr2-35076p9 | 42 | 3378 | 3379 |
| v4chr2-35253m8 | 42 | 3380 | 3381 |
| v4chr2-36549p16 | 42 | 3382 | 3383 |
| v4chr2-36989m29 | 42 | 3384 | 3385 |
| v4chr2-37212p17 | 42 | 3386 | 3387 |
| v4chr2-37796m9 | 42 | 3388 | 3389 |
| v4chr2-38282m9 | 42 | 3390 | 3391 |
| v4chr2-38312p19 | 42 | 3392 | 3393 |
| v4chr2-38585m6 | 42 | 3394 | 3395 |
| v4chr2-38667p7 | 42 | 3396 | 3397 |
| v4chr2-38754p5 | 42 | 3398 | 3399 |
| v4chr2-39211m22 | 42 | 3400 | 3401 |
| v4chr2-39304m25 | 42 | 3402 | 3403 |
| v4chr2-40684m8 | 42 | 3404 | 3405 |
| v4chr2-40793p16 | 42 | 3406 | 3407 |
| v4chr2-41392m10 | 42 | 3408 | 3409 |
| v4chr2-41432m15 | 42 | 3410 | 3411 |
| v4chr2-41926p13 | 42 | 3412 | 3413 |
| v4chr2-42468m20 | 42 | 3414 | 3415 |
| v4chr2-43602p2 | 42 | 3416 | 3417 |
| v4chr2-43621p7 | 42 | 3418 | 3419 |
| v4chr2-443m19 | 42 | 3420 | 3421 |
| v4chr2-44624p5 | 42 | 3422 | 3423 |
| v4chr2-44636p25 | 42 | 3424 | 3425 |
| v4chr2-44925p18 | 42 | 3426 | 3427 |
| v4chr2-46397p19 | 42 | 3428 | 3429 |
| v4chr2-4732p21 | 42 | 3430 | 3431 |
| v4chr2-47648p15 | 42 | 3432 | 3433 |
| v4chr2-48304m28 | 42 | 3434 | 3435 |
| v4chr2-48586m20 | 42 | 3436 | 3437 |
| v4chr2-488m8 | 42 | 3438 | 3439 |
| v4chr2-49839p10 | 42 | 3440 | 3441 |
| v4chr2-49981m17 | 42 | 3442 | 3443 |
| v4chr2-50031m18 | 42 | 3444 | 3445 |
| v4chr2-50308m37 | 42 | 3446 | 3447 |
| v4chr2-50392m11 | 42 | 3448 | 3449 |
| v4chr2-51125m9 | 42 | 3450 | 3451 |
| v4chr2-52108m6 | 42 | 3452 | 3453 |
| v4chr2-52347p26 | 42 | 3454 | 3455 |
| v4chr2-53120p3 | 42 | 3456 | 3457 |
| v4chr2-53216p22 | 42 | 3458 | 3459 |
| v4chr2-53320m37 | 42 | 3460 | 3461 |
| v4chr2-5332p16 | 42 | 3462 | 3463 |
| v4chr2-53427m9 | 42 | 3464 | 3465 |
| v4chr2-53620m3 | 42 | 3466 | 3467 |
| v4chr2-5634m9 | 42 | 3468 | 3469 |
| v4chr2-56362m1 | 42 | 3470 | 3471 |
| v4chr2-5648p8 | 42 | 3472 | 3473 |
| v4chr2-56760p4 | 42 | 3474 | 3475 |
| v4chr2-57437m11 | 42 | 3476 | 3477 |
| v4chr2-5790p34 | 42 | 3478 | 3479 |
| v4chr2-58216m2 | 42 | 3480 | 3481 |
| v4chr2-58230p3 | 42 | 3482 | 3483 |
| v4chr2-59578m7 | 42 | 3484 | 3485 |
| v4chr2-61345p9 | 42 | 3486 | 3487 |
| v4chr2-61368m12 | 42 | 3488 | 3489 |
| v4chr2-61514p20 | 42 | 3490 | 3491 |
| v4chr2-61574m11 | 42 | 3492 | 3493 |
| v4chr2-61611p11 | 42 | 3494 | 3495 |
| v4chr2-62103m23 | 42 | 3496 | 3497 |
| v4chr2-62147m17 | 42 | 3498 | 3499 |
| v4chr2-6364p13 | 42 | 3500 | 3501 |
| v4chr2-64608m20 | 42 | 3502 | 3503 |
| v4chr2-64794p8 | 42 | 3504 | 3505 |
| v4chr2-649p8 | 42 | 3506 | 3507 |
| v4chr2-65300p1 | 42 | 3508 | 3509 |
| v4chr2-65472m6 | 42 | 3510 | 3511 |
| v4chr2-65542m18 | 42 | 3512 | 3513 |
| v4chr2-66342m15 | 42 | 3514 | 3515 |
| v4chr2-66557p34 | 42 | 3516 | 3517 |
| v4chr2-67093p48 | 42 | 3518 | 3519 |
| v4chr2-67329p4 | 42 | 3520 | 3521 |
| v4chr2-68277m16 | 42 | 3522 | 3523 |
| v4chr2-68337p25 | 42 | 3524 | 3525 |
| v4chr2-68387m8 | 42 | 3526 | 3527 |
| v4chr2-69203p16 | 42 | 3528 | 3529 |
| v4chr2-69588m3 | 42 | 3530 | 3531 |
| v4chr2-71832p9 | 42 | 3532 | 3533 |
| v4chr2-72231p13 | 42 | 3534 | 3535 |
| v4chr2-73621p47 | 42 | 3536 | 3537 |
| v4chr2-74377m5 | 42 | 3538 | 3539 |
| v4chr2-74416p2 | 42 | 3540 | 3541 |
| v4chr2-75907m11 | 42 | 3542 | 3543 |
| v4chr2-75955p14 | 42 | 3544 | 3545 |
| v4chr2-77542p16 | 42 | 3546 | 3547 |
| v4chr2-8742p8 | 42 | 3548 | 3549 |
| v4chr2-9010m22 | 42 | 3550 | 3551 |
| v4chr2-9257m35 | 42 | 3552 | 3553 |
| v4chr2-9336m6 | 42 | 3554 | 3555 |
| v4chr2-9342p21 | 42 | 3556 | 3557 |
| v4chr2-9904m2 | 42 | 3558 | 3559 |
| v4chr3-10344m19 | 42 | 3560 | 3561 |
| v4chr3-10454m3 | 42 | 3562 | 3563 |
| v4chr3-10548m30 | 42 | 3564 | 3565 |
| v4chr3-10587m14 | 42 | 3566 | 3567 |
| v4chr3-11063p33 | 42 | 3568 | 3569 |
| v4chr3-11492p20 | 42 | 3570 | 3571 |
| v4chr3-11548p4 | 42 | 3572 | 3573 |
| v4chr3-11568m6 | 42 | 3574 | 3575 |
| v4chr3-12372m17 | 42 | 3576 | 3577 |
| v4chr3-12531p3 | 42 | 3578 | 3579 |
| v4chr3-12746m13 | 42 | 3580 | 3581 |
| v4chr3-12826m11 | 42 | 3582 | 3583 |
| v4chr3-12840p22 | 42 | 3584 | 3585 |
| v4chr3-12886p21 | 42 | 3586 | 3587 |
| v4chr3-12939m23 | 42 | 3588 | 3589 |
| v4chr3-13198p40 | 42 | 3590 | 3591 |
| v4chr3-13566p53 | 42 | 3592 | 3593 |
| v4chr3-1356m10 | 42 | 3594 | 3595 |
| v4chr3-13634m10 | 42 | 3596 | 3597 |
| v4chr3-13834m42 | 42 | 3598 | 3599 |
| v4chr3-14051m28 | 42 | 3600 | 3601 |
| v4chr3-14078p25 | 42 | 3602 | 3603 |
| v4chr3-14323m19 | 42 | 3604 | 3605 |
| v4chr3-14421m13 | 42 | 3606 | 3607 |
| v4chr3-14653p3 | 42 | 3608 | 3609 |
| v4chr3-14850p11 | 42 | 3610 | 3611 |
| v4chr3-1528p4 | 42 | 3612 | 3613 |
| v4chr3-15672m3 | 42 | 3614 | 3615 |
| v4chr3-15769p23 | 42 | 3616 | 3617 |
| v4chr3-16434m8 | 42 | 3618 | 3619 |
| v4chr3-16833p16 | 42 | 3620 | 3621 |
| v4chr3-17731p4 | 42 | 3622 | 3623 |
| v4chr3-18104m12 | 42 | 3624 | 3625 |
| v4chr3-18603p5 | 42 | 3626 | 3627 |
| v4chr3-19121m7 | 42 | 3628 | 3629 |
| v4chr3-19326m10 | 42 | 3630 | 3631 |
| v4chr3-19385m11 | 42 | 3632 | 3633 |
| v4chr3-20018m6 | 42 | 3634 | 3635 |
| v4chr3-20066p14 | 42 | 3636 | 3637 |
| v4chr3-20085p18 | 42 | 3638 | 3639 |
| v4chr3-20146m34 | 42 | 3640 | 3641 |
| v4chr3-20361p5 | 42 | 3642 | 3643 |
| v4chr3-21141p17 | 42 | 3644 | 3645 |
| v4chr3-21179m8 | 42 | 3646 | 3647 |
| v4chr3-22109p11 | 42 | 3648 | 3649 |
| v4chr3-22147p19 | 42 | 3650 | 3651 |
| v4chr3-2217m1 | 42 | 3652 | 3653 |
| v4chr3-22199m3 | 42 | 3654 | 3655 |
| v4chr3-2225p17 | 42 | 3656 | 3657 |
| v4chr3-22365p5 | 42 | 3658 | 3659 |
| v4chr3-22385p27 | 42 | 3660 | 3661 |
| v4chr3-22443m27 | 42 | 3662 | 3663 |
| v4chr3-22600m14 | 42 | 3664 | 3665 |
| v4chr3-23089p48 | 42 | 3666 | 3667 |
| v4chr3-2313p1 | 42 | 3668 | 3669 |
| v4chr3-23159m11 | 42 | 3670 | 3671 |
| v4chr3-23166p21 | 42 | 3672 | 3673 |
| v4chr3-24922m2 | 42 | 3674 | 3675 |
| v4chr3-25151p21 | 42 | 3676 | 3677 |
| v4chr3-25190p20 | 42 | 3678 | 3679 |
| v4chr3-2531m23 | 42 | 3680 | 3681 |
| v4chr3-25388p3 | 42 | 3682 | 3683 |
| v4chr3-25411m16 | 42 | 3684 | 3685 |
| v4chr3-25417p11 | 42 | 3686 | 3687 |
| v4chr3-2614m19 | 42 | 3688 | 3689 |
| v4chr3-27104p8 | 42 | 3690 | 3691 |
| v4chr3-27122m8 | 42 | 3692 | 3693 |
| v4chr3-27145m17 | 42 | 3694 | 3695 |
| v4chr3-27151p24 | 42 | 3696 | 3697 |
| v4chr3-27221m42 | 42 | 3698 | 3699 |
| v4chr3-27407m28 | 42 | 3700 | 3701 |
| v4chr3-27466m40 | 42 | 3702 | 3703 |
| v4chr3-27972m7 | 42 | 3704 | 3705 |
| v4chr3-2799p18 | 42 | 3706 | 3707 |
| v4chr3-28121m9 | 42 | 3708 | 3709 |
| v4chr3-28148m16 | 42 | 3710 | 3711 |
| v4chr3-28159m7 | 42 | 3712 | 3713 |
| v4chr3-28161p14 | 42 | 3714 | 3715 |
| v4chr3-28186m5 | 42 | 3716 | 3717 |
| v4chr3-28240p27 | 42 | 3718 | 3719 |
| v4chr3-28398m11 | 42 | 3720 | 3721 |
| v4chr3-28406p21 | 42 | 3722 | 3723 |
| v4chr3-28477m38 | 42 | 3724 | 3725 |
| v4chr3-28557m8 | 42 | 3726 | 3727 |
| v4chr3-2919p6 | 42 | 3728 | 3729 |
| v4chr3-30201m43 | 42 | 3730 | 3731 |
| v4chr3-30243m30 | 42 | 3732 | 3733 |
| v4chr3-30340m26 | 42 | 3734 | 3735 |
| v4chr3-30369p2 | 42 | 3736 | 3737 |
| v4chr3-31028m44 | 42 | 3738 | 3739 |
| v4chr3-31184p9 | 42 | 3740 | 3741 |
| v4chr3-31213m17 | 42 | 3742 | 3743 |
| v4chr3-31781p21 | 42 | 3744 | 3745 |
| v4chr3-32208m6 | 42 | 3746 | 3747 |
| v4chr3-32304m30 | 42 | 3748 | 3749 |
| v4chr3-32337m14 | 42 | 3750 | 3751 |
| v4chr3-33100p9 | 42 | 3752 | 3753 |
| v4chr3-33329m10 | 42 | 3754 | 3755 |
| v4chr3-35262p33 | 42 | 3756 | 3757 |
| v4chr3-35520m2 | 42 | 3758 | 3759 |
| v4chr3-36156m4 | 42 | 3760 | 3761 |
| v4chr3-36261p13 | 42 | 3762 | 3763 |
| v4chr3-36971m8 | 42 | 3764 | 3765 |
| v4chr3-37286p16 | 42 | 3766 | 3767 |
| v4chr3-37307p13 | 42 | 3768 | 3769 |
| v4chr3-37617p28 | 42 | 3770 | 3771 |
| v4chr3-37916m8 | 42 | 3772 | 3773 |
| v4chr3-38212m11 | 42 | 3774 | 3775 |
| v4chr3-38363m11 | 42 | 3776 | 3777 |
| v4chr3-3869m33 | 42 | 3778 | 3779 |
| v4chr3-39241p25 | 42 | 3780 | 3781 |
| v4chr3-39272p8 | 42 | 3782 | 3783 |
| v4chr3-3927m11 | 42 | 3784 | 3785 |
| v4chr3-39636m17 | 42 | 3786 | 3787 |
| v4chr3-40025p16 | 42 | 3788 | 3789 |
| v4chr3-40098p18 | 42 | 3790 | 3791 |
| v4chr3-4035p15 | 42 | 3792 | 3793 |
| v4chr3-40363m30 | 42 | 3794 | 3795 |
| v4chr3-40923p12 | 42 | 3796 | 3797 |
| v4chr3-40937p8 | 42 | 3798 | 3799 |
| v4chr3-41524p19 | 42 | 3800 | 3801 |
| v4chr3-41718p8 | 42 | 3802 | 3803 |
| v4chr3-42458p10 | 42 | 3804 | 3805 |
| v4chr3-42861p2 | 42 | 3806 | 3807 |
| v4chr3-43326p3 | 42 | 3808 | 3809 |
| v4chr3-4362m10 | 42 | 3810 | 3811 |
| v4chr3-44129m4 | 42 | 3812 | 3813 |
| v4chr3-44302m19 | 42 | 3814 | 3815 |
| v4chr3-44694p8 | 42 | 3816 | 3817 |
| v4chr3-44964p16 | 42 | 3818 | 3819 |
| v4chr3-45321p19 | 42 | 3820 | 3821 |
| v4chr3-46034p1 | 42 | 3822 | 3823 |
| v4chr3-46363p19 | 42 | 3824 | 3825 |
| v4chr3-46545m8 | 42 | 3826 | 3827 |
| v4chr3-46745m12 | 42 | 3828 | 3829 |
| v4chr3-47694p4 | 42 | 3830 | 3831 |
| v4chr3-47983m5 | 42 | 3832 | 3833 |
| v4chr3-48810p16 | 42 | 3834 | 3835 |
| v4chr3-49652m32 | 42 | 3836 | 3837 |
| v4chr3-49754m10 | 42 | 3838 | 3839 |
| v4chr3-4987m7 | 42 | 3840 | 3841 |
| v4chr3-50017p22 | 42 | 3842 | 3843 |
| v4chr3-50118m7 | 42 | 3844 | 3845 |
| v4chr3-50582m9 | 42 | 3846 | 3847 |
| v4chr3-50648p20 | 42 | 3848 | 3849 |
| v4chr3-5186p19 | 42 | 3850 | 3851 |
| v4chr3-53200p10 | 42 | 3852 | 3853 |
| v4chr3-53302m12 | 42 | 3854 | 3855 |
| v4chr3-5332p14 | 42 | 3856 | 3857 |
| v4chr3-53440m24 | 42 | 3858 | 3859 |
| v4chr3-53592p9 | 42 | 3860 | 3861 |
| v4chr3-53627m3 | 42 | 3862 | 3863 |
| v4chr3-54171p7 | 42 | 3864 | 3865 |
| v4chr3-54247p9 | 42 | 3866 | 3867 |
| v4chr3-54310p14 | 42 | 3868 | 3869 |
| v4chr3-5575m9 | 42 | 3870 | 3871 |
| v4chr3-6303p6 | 42 | 3872 | 3873 |
| v4chr3-6314p9 | 42 | 3874 | 3875 |
| v4chr3-6324p21 | 42 | 3876 | 3877 |
| v4chr3-6441p10 | 42 | 3878 | 3879 |
| v4chr3-6707m4 | 42 | 3880 | 3881 |
| v4chr3-7293p19 | 42 | 3882 | 3883 |
| v4chr3-7320p26 | 42 | 3884 | 3885 |
| v4chr3-7391m18 | 42 | 3886 | 3887 |
| v4chr3-758p13 | 42 | 3888 | 3889 |
| v4chr3-828p12 | 42 | 3890 | 3891 |
| v4chr3-8695p20 | 42 | 3892 | 3893 |
| v4chr3-8741p25 | 42 | 3894 | 3895 |
| v4chr3-9391p20 | 42 | 3896 | 3897 |
| v4chr3-9922p21 | 42 | 3898 | 3899 |
| v4chr4-11120p3 | 42 | 3900 | 3901 |
| v4chr4-11996m3 | 42 | 3902 | 3903 |
| v4chr4-12337m24 | 42 | 3904 | 3905 |
| v4chr4-12386m13 | 42 | 3906 | 3907 |
| v4chr4-12435m13 | 42 | 3908 | 3909 |
| v4chr4-12795m13 | 42 | 3910 | 3911 |
| v4chr4-1301p6 | 42 | 3912 | 3913 |
| v4chr4-13057m27 | 42 | 3914 | 3915 |
| v4chr4-13062p17 | 42 | 3916 | 3917 |
| v4chr4-13296p67 | 42 | 3918 | 3919 |
| v4chr4-13451m28 | 42 | 3920 | 3921 |
| v4chr4-14468m45 | 42 | 3922 | 3923 |
| v4chr4-15162p16 | 42 | 3924 | 3925 |
| v4chr4-15240p20 | 42 | 3926 | 3927 |
| v4chr4-15305m25 | 42 | 3928 | 3929 |
| v4chr4-15366p10 | 42 | 3930 | 3931 |
| v4chr4-1546m11 | 42 | 3932 | 3933 |
| v4chr4-15980p28 | 42 | 3934 | 3935 |
| v4chr4-16342p54 | 42 | 3936 | 3937 |
| v4chr4-16421m14 | 42 | 3938 | 3939 |
| v4chr4-17332m11 | 42 | 3940 | 3941 |
| v4chr4-17540m25 | 42 | 3942 | 3943 |
| v4chr4-17773m11 | 42 | 3944 | 3945 |
| v4chr4-1783m4 | 42 | 3946 | 3947 |
| v4chr4-17868m18 | 42 | 3948 | 3949 |
| v4chr4-18331m8 | 42 | 3950 | 3951 |
| v4chr4-18342p15 | 42 | 3952 | 3953 |
| v4chr4-18907m19 | 42 | 3954 | 3955 |
| v4chr4-19051m4 | 42 | 3956 | 3957 |
| v4chr4-19250m37 | 42 | 3958 | 3959 |
| v4chr4-19463m16 | 42 | 3960 | 3961 |
| v4chr4-19510p23 | 42 | 3962 | 3963 |
| v4chr4-19926p25 | 42 | 3964 | 3965 |
| v4chr4-20003m12 | 42 | 3966 | 3967 |
| v4chr4-20130m2 | 42 | 3968 | 3969 |
| v4chr4-20172p36 | 42 | 3970 | 3971 |
| v4chr4-20215p67 | 42 | 3972 | 3973 |
| v4chr4-20578m19 | 42 | 3974 | 3975 |
| v4chr4-20847m13 | 42 | 3976 | 3977 |
| v4chr4-20986p43 | 42 | 3978 | 3979 |
| v4chr4-23276m56 | 42 | 3980 | 3981 |
| v4chr4-23337m16 | 42 | 3982 | 3983 |
| v4chr4-24409p16 | 42 | 3984 | 3985 |
| v4chr4-24826p4 | 42 | 3986 | 3987 |
| v4chr4-25156m34 | 42 | 3988 | 3989 |
| v4chr4-26126p5 | 42 | 3990 | 3991 |
| v4chr4-26368p1 | 42 | 3992 | 3993 |
| v4chr4-27106m25 | 42 | 3994 | 3995 |
| v4chr4-28509m8 | 42 | 3996 | 3997 |
| v4chr4-28758m14 | 42 | 3998 | 3999 |
| v4chr4-29088p37 | 42 | 4000 | 4001 |
| v4chr4-29189p10 | 42 | 4002 | 4003 |
| v4chr4-29355m43 | 42 | 4004 | 4005 |
| v4chr4-29692p10 | 42 | 4006 | 4007 |
| v4chr4-29836p83 | 42 | 4008 | 4009 |
| v4chr4-30196p14 | 42 | 4010 | 4011 |
| v4chr4-30233p17 | 42 | 4012 | 4013 |
| v4chr4-30511m26 | 42 | 4014 | 4015 |
| v4chr4-30707p15 | 42 | 4016 | 4017 |
| v4chr4-30964p18 | 42 | 4018 | 4019 |
| v4chr4-30997m9 | 42 | 4020 | 4021 |
| v4chr4-31228p17 | 42 | 4022 | 4023 |
| v4chr4-32250p8 | 42 | 4024 | 4025 |
| v4chr4-32607m5 | 42 | 4026 | 4027 |
| v4chr4-32619p12 | 42 | 4028 | 4029 |
| v4chr4-32872m38 | 42 | 4030 | 4031 |
| v4chr4-33224m9 | 42 | 4032 | 4033 |
| v4chr4-33425m7 | 42 | 4034 | 4035 |
| v4chr4-33447p11 | 42 | 4036 | 4037 |
| v4chr4-33594p12 | 42 | 4038 | 4039 |
| v4chr4-34490p14 | 42 | 4040 | 4041 |
| v4chr4-35111p24 | 42 | 4042 | 4043 |
| v4chr4-35251m45 | 42 | 4044 | 4045 |
| v4chr4-36233m20 | 42 | 4046 | 4047 |
| v4chr4-36238p2 | 42 | 4048 | 4049 |
| v4chr4-36362m6 | 42 | 4050 | 4051 |
| v4chr4-36659p7 | 42 | 4052 | 4053 |
| v4chr4-36753p10 | 42 | 4054 | 4055 |
| v4chr4-37052m11 | 42 | 4056 | 4057 |
| v4chr4-37235m27 | 42 | 4058 | 4059 |
| v4chr4-37281m32 | 42 | 4060 | 4061 |
| v4chr4-37298m8 | 42 | 4062 | 4063 |
| v4chr4-3743m18 | 42 | 4064 | 4065 |
| v4chr4-37770p18 | 42 | 4066 | 4067 |
| v4chr4-38372p4 | 42 | 4068 | 4069 |
| v4chr4-39037m32 | 42 | 4070 | 4071 |
| v4chr4-39288m30 | 42 | 4072 | 4073 |
| v4chr4-39444p20 | 42 | 4074 | 4075 |
| v4chr4-39894p22 | 42 | 4076 | 4077 |
| v4chr4-40736m28 | 42 | 4078 | 4079 |
| v4chr4-41352m46 | 42 | 4080 | 4081 |
| v4chr4-4185p41 | 42 | 4082 | 4083 |
| v4chr4-41976p14 | 42 | 4084 | 4085 |
| v4chr4-42026p13 | 42 | 4086 | 4087 |
| v4chr4-42260m10 | 42 | 4088 | 4089 |
| v4chr4-43037m19 | 42 | 4090 | 4091 |
| v4chr4-4465m35 | 42 | 4092 | 4093 |
| v4chr4-45362m14 | 42 | 4094 | 4095 |
| v4chr4-45935m18 | 42 | 4096 | 4097 |
| v4chr4-47755p1 | 42 | 4098 | 4099 |
| v4chr4-48311p2 | 42 | 4100 | 4101 |
| v4chr4-486p10 | 42 | 4102 | 4103 |
| v4chr4-5175p11 | 42 | 4104 | 4105 |
| v4chr4-5359p11 | 42 | 4106 | 4107 |
| v4chr4-5467m34 | 42 | 4108 | 4109 |
| v4chr4-6424p6 | 42 | 4110 | 4111 |
| v4chr4-8587m11 | 42 | 4112 | 4113 |
| v4chr5-10459m21 | 42 | 4114 | 4115 |
| v4chr5-12391p7 | 42 | 4116 | 4117 |
| v4chr5-12403p31 | 42. | 4118 | 4119 |
| v4chr5-12443p12 | 42 | 4120 | 4121 |
| v4chr5-13535m10 | 42 | 4122 | 4123 |
| v4chr5-13652p3 | 42 | 4124 | 4125 |
| v4chr5-13817p10 | 42 | 4126 | 4127 |
| v4chr5-13877m8 | 42 | 4128 | 4129 |
| v4chr5-14342p17 | 42 | 4130 | 4131 |
| v4chr5-14394p72 | 42 | 4132 | 4133 |
| v4chr5-14534p18 | 42 | 4134 | 4135 |
| v4chr5-15137p19 | 42 | 4136 | 4137 |
| v4chr5-15270p7 | 42 | 4138 | 4139 |
| v4chr5-15442m71 | 42 | 4140 | 4141 |
| v4chr5-16473p20 | 42 | 4142 | 4143 |
| v4chr5-16565p21 | 42 | 4144 | 4145 |
| v4chr5-1739p19 | 42 | 4146 | 4147 |
| v4chr5-17673p3 | 42 | 4148 | 4149 |
| v4chr5-181m5 | 42 | 4150 | 4151 |
| v4chr5-18269p31 | 42 | 4152 | 4153 |
| v4chr5-19025m15 | 42 | 4154 | 4155 |
| v4chr5-19284p37 | 42 | 4156 | 4157 |
| v4chr5-19492p42 | 42 | 4158 | 4159 |
| v4chr5-19654m19 | 42 | 4160 | 4161 |
| v4chr5-19887m13 | 42 | 4162 | 4163 |
| v4chr5-20142p15 | 42 | 4164 | 4165 |
| v4chr5-20181p16 | 42 | 4166 | 4167 |
| v4chr5-2057p16 | 42 | 4168 | 4169 |
| v4chr5-21783p23 | 42 | 4170 | 4171 |
| v4chr5-22151p29 | 42 | 4172 | 4173 |
| v4chr5-22213m20 | 42 | 4174 | 4175 |
| v4chr5-22682p11 | 42 | 4176 | 4177 |
| v4chr5-23246m36 | 42 | 4178 | 4179 |
| v4chr5-23306p32 | 42 | 4180 | 4181 |
| v4chr5-23553p26 | 42 | 4182 | 4183 |
| v4chr5-24125m16 | 42 | 4184 | 4185 |
| v4chr5-24156p13 | 42 | 4186 | 4187 |
| v4chr5-24362m29 | 42 | 4188 | 4189 |
| v4chr5-25317m45 | 42 | 4190 | 4191 |
| v4chr5-25829m34 | 42 | 4192 | 4193 |
| v4chr5-26126p9 | 42 | 4194 | 4195 |
| v4chr5-26633m56 | 42 | 4196 | 4197 |
| v4chr5-26750p11 | 42 | 4198 | 4199 |
| v4chr5-27100m15 | 42 | 4200 | 4201 |
| v4chr5-27295m3 | 42 | 4202 | 4203 |
| v4chr5-27375m4 | 42 | 4204 | 4205 |
| v4chr5-29157p11 | 42 | 4206 | 4207 |
| v4chr5-31209p11 | 42 | 4208 | 4209 |
| v4chr5-31230p5 | 42 | 4210 | 4211 |
| v4chr5-31238p3 | 42 | 4212 | 4213 |
| v4chr5-31250p19 | 42 | 4214 | 4215 |
| v4chr5-31291m10 | 42 | 4216 | 4217 |
| v4chr5-31382p11 | 42 | 4218 | 4219 |
| v4chr5-32233p14 | 42 | 4220 | 4221 |
| v4chr5-32250p35 | 42 | 4222 | 4223 |
| v4chr5-32315m6 | 42 | 4224 | 4225 |
| v4chr5-32648p11 | 42 | 4226 | 4227 |
| v4chr5-34247p7 | 42 | 4228 | 4229 |
| v4chr5-34963p27 | 42 | 4230 | 4231 |
| v4chr5-3500m15 | 42 | 4232 | 4233 |
| v4chr5-35352p79 | 42 | 4234 | 4235 |
| v4chr5-36554m16 | 42 | 4236 | 4237 |
| v4chr5-36669p8 | 42 | 4238 | 4239 |
| v4chr5-36775p13 | 42 | 4240 | 4241 |
| v4chr5-36860m11 | 42 | 4242 | 4243 |
| v4chr5-36874m5 | 42 | 4244 | 4245 |
| v4chr5-38215p6 | 42 | 4246 | 4247 |
| v4chr5-38269m19 | 42 | 4248 | 4249 |
| v4chr5-38278p43 | 42 | 4250 | 4251 |
| v4chr5-38390m8 | 42 | 4252 | 4253 |
| v4chr5-38634m2 | 42 | 4254 | 4255 |
| v4chr5-39223p9 | 42 | 4256 | 4257 |
| v4chr5-39281m21 | 42 | 4258 | 4259 |
| v4chr5-39673m6 | 42 | 4260 | 4261 |
| v4chr5-40386m2 | 42 | 4262 | 4263 |
| v4chr5-40492p18 | 42 | 4264 | 4265 |
| v4chr5-40776p52 | 42 | 4266 | 4267 |
| v4chr5-42392m26 | 42 | 4268 | 4269 |
| v4chr5-42458p10 | 42 | 4270 | 4271 |
| v4chr5-43050m5 | 42 | 4272 | 4273 |
| v4chr5-43132m3 | 42 | 4274 | 4275 |
| v4chr5-43753p82 | 42 | 4276 | 4277 |
| v4chr5-44538p6 | 42 | 4278 | 4279 |
| v4chr5-44596p14 | 42 | 4280 | 4281 |
| v4chr5-45077p3 | 42 | 4282 | 4283 |
| v4chr5-45670m11 | 42 | 4284 | 4285 |
| v4chr5-47013m15 | 42 | 4286 | 4287 |
| v4chr5-4775m5 | 42 | 4288 | 4289 |
| v4chr5-48118p14 | 42 | 4290 | 4291 |
| v4chr5-5319p2 | 42 | 4292 | 4293 |
| v4chr5-6376m15 | 42 | 4294 | 4295 |
| v4chr5-695p39 | 42 | 4296 | 4297 |
| v4chr5-8065m15 | 42 | 4298 | 4299 |
| v4chr5-8127m50 | 42 | 4300 | 4301 |
| v4chr5-8286p28 | 42 | 4302 | 4303 |
| v4chr5-9441m24 | 42 | 4304 | 4305 |
| v4chr6a-1025m5 | 42 | 4306 | 4307 |
| v4chr6a-1065m3 | 42 | 4308 | 4309 |
| v4chr6a-10711p22 | 42 | 4310 | 4311 |
| v4chr6a-11039p36 | 42 | 4312 | 4313 |
| v4chr6a-11733p2 | 42 | 4314 | 4315 |
| v4chr6a-11872p13 | 42 | 4316 | 4317 |
| v4chr6a-13424m9 | 42 | 4318 | 4319 |
| v4chr6a-13483p50 | 42 | 4320 | 4321 |
| v4chr6a-14606p4 | 42 | 4322 | 4323 |
| v4chr6a-15532m7 | 42 | 4324 | 4325 |
| v4chr6a-16336m27 | 42 | 4326 | 4327 |
| v4chr6a-16440p13 | 42 | 4328 | 4329 |
| v4chr6a-16778m6 | 42 | 4330 | 4331 |
| v4chr6a-16779p9 | 42 | 4332 | 4333 |
| v4chr6a-17311m11 | 42 | 4334 | 4335 |
| v4chr6a-17643p32 | 42 | 4336 | 4337 |
| v4chr6a-17691p11 | 42 | 4338 | 4339 |
| v4chr6a-18292m6 | 42 | 4340 | 4341 |
| v4chr6a-18833p14 | 42 | 4342 | 4343 |
| v4chr6a-19043p23 | 42 | 4344 | 4345 |
| v4chr6a-19382m18 | 42 | 4346 | 4347 |
| v4chr6a-19620m50 | 42 | 4348 | 4349 |
| v4chr6a-19781m40 | 42 | 4350 | 4351 |
| v4chr6a-19937m9 | 42 | 4352 | 4353 |
| v4chr6a-20069m18 | 42 | 4354 | 4355 |
| v4chr6a-20132m14 | 42 | 4356 | 4357 |
| v4chr6a-20163p16 | 42 | 4358 | 4359 |
| v4chr6a-20234m7 | 42 | 4360 | 4361 |
| v4chr6a-20332p33 | 42 | 4362 | 4363 |
| v4chr6a-20387m14 | 42 | 4364 | 4365 |
| v4chr6a-23727m13 | 42 | 4366 | 4367 |
| v4chr6a-24323p25 | 42 | 4368 | 4369 |
| v4chr6a-24630p17 | 42 | 4370 | 4371 |
| v4chr6a-25152m27 | 42 | 4372 | 4373 |
| v4chr6a-2597p21 | 42 | 4374 | 4375 |
| v4chr6a-27402m11 | 42 | 4376 | 4377 |
| v4chr6a-28085p24 | 42 | 4378 | 4379 |
| v4chr6a-28139m18 | 42 | 4380 | 4381 |
| v4chr6a-28149m4 | 42 | 4382 | 4383 |
| v4chr6a-28326p9 | 42 | 4384 | 4385 |
| v4chr6a-28495m18 | 42 | 4386 | 4387 |
| v4chr6a-29050p22 | 42 | 4388 | 4389 |
| v4chr6a-29102m10 | 42 | 4390 | 4391 |
| v4chr6a-29199p41 | 42 | 4392 | 4393 |
| v4chr6a-30212p16 | 42 | 4394 | 4395 |
| v4chr6a-30307m31 | 42 | 4396 | 4397 |
| v4chr6a-30326m15 | 42 | 4398 | 4399 |
| v4chr6a-30424m27 | 42 | 4400 | 4401 |
| v4chr6a-31708p14 | 42 | 4402 | 4403 |
| v4chr6a-32204m16 | 42 | 4404 | 4405 |
| v4chr6a-33248m18 | 42 | 4406 | 4407 |
| v4chr6a-33313m37 | 42 | 4408 | 4409 |
| v4chr6a-3367p12 | 42 | 4410 | 4411 |
| v4chr6a-33915p16 | 42 | 4412 | 4413 |
| v4chr6a-34221p9 | 42 | 4414 | 4415 |
| v4chr6a-34320p16 | 42 | 4416 | 4417 |
| v4chr6a-34956p34 | 42 | 4418 | 4419 |
| v4chr6a-35286m21 | 42 | 4420 | 4421 |
| v4chr6a-3552m33 | 42 | 4422 | 4423 |
| v4chr6a-35540p6 | 42 | 4424 | 4425 |
| v4chr6a-35805p4 | 42 | 4426 | 4427 |
| v4chr6a-35820m2 | 42 | 4428 | 4429 |
| v4chr6a-36134m39 | 42 | 4430 | 4431 |
| v4chr6a-3642p12 | 42 | 4432 | 4433 |
| v4chr6a-36740p5 | 42 | 4434 | 4435 |
| v4chr6a-3680p31 | 42 | 4436 | 4437 |
| v4chr6a-37196p4 | 42 | 4438 | 4439 |
| v4chr6a-462p3 | 42 | 4440 | 4441 |
| v4chr6a-6404m4 | 42 | 4442 | 4443 |
| v4chr6a-8117p4 | 42 | 4444 | 4445 |
| v4chr6a-8372m6 | 42 | 4446 | 4447 |
| v4chr6a-8993p3 | 42 | 4448 | 4449 |
| v4chr6b-10295p3 | 42 | 4450 | 4451 |
| v4chr6b-10368m10 | 42 | 4452 | 4453 |
| v4chr6b-10403p11 | 42 | 4454 | 4455 |
| v4chr6b-11223m26 | 42 | 4456 | 4457 |
| v4chr6b-11401p20 | 42 | 4458 | 4459 |
| v4chr6b-11703p2 | 42 | 4460 | 4461 |
| v4chr6b-126m10 | 42 | 4462 | 4463 |
| v4chr6b-12930m9 | 42 | 4464 | 4465 |
| v4chr6b-13126m4 | 42 | 4466 | 4467 |
| v4chr6b-13377m28 | 42 | 4468 | 4469 |
| v4chr6b-13501p13 | 42 | 4470 | 4471 |
| v4chr6b-1375p16 | 42 | 4472 | 4473 |
| v4chr6b-13828p12 | 42 | 4474 | 4475 |
| v4chr6b-14111p12 | 42 | 4476 | 4477 |
| v4chr6b-14355p7 | 42 | 4478 | 4479 |
| v4chr6b-14529m15 | 42 | 4480 | 4481 |
| v4chr6b-15874p8 | 42 | 4482 | 4483 |
| v4chr6b-241m9 | 42 | 4484 | 4485 |
| v4chr6b-2715m27 | 42 | 4486 | 4487 |
| v4chr6b-2778p5 | 42 | 4488 | 4489 |
| v4chr6b-4296m48 | 42 | 4490 | 4491 |
| v4chr6b-4399m10 | 42 | 4492 | 4493 |
| v4chr6b-5362m10 | 42 | 4494 | 4495 |
| v4chr6b-6327p26 | 42 | 4496 | 4497 |
| v4chr6b-8143m4 | 42 | 4498 | 4499 |
| v4chr7-10322m11 | 42 | 4500 | 4501 |
| v4chr7-10873m20 | 42 | 4502 | 4503 |
| v4chr7-11227p20 | 42 | 4504 | 4505 |
| v4chr7-11916p25 | 42 | 4506 | 4507 |
| v4chr7-12651m28 | 42 | 4508 | 4509 |
| v4chr7-12852p21 | 42 | 4510 | 4511 |
| v4chr7-13623m34 | 42 | 4512 | 4513 |
| v4chr7-13941p17 | 42 | 4514 | 4515 |
| v4chr7-14302p6 | 42 | 4516 | 4517 |
| v4chr7-14356m36 | 42 | 4518 | 4519 |
| v4chr7-14523p48 | 42 | 4520 | 4521 |
| v4chr7-14937p2 | 42 | 4522 | 4523 |
| v4chr7-1504m12 | 42 | 4524 | 4525 |
| v4chr7-15914p51 | 42 | 4526 | 4527 |
| v4chr7-15997m16 | 42 | 4528 | 4529 |
| v4chr7-16039m21 | 42 | 4530 | 4531 |
| v4chr7-16135m15 | 42 | 4532 | 4533 |
| v4chr7-17550p5 | 42 | 4534 | 4535 |
| v4chr7-17560p5 | 42 | 4536 | 4537 |
| v4chr7-17615m10 | 42 | 4538 | 4539 |
| v4chr7-18388m25 | 42 | 4540 | 4541 |
| v4chr7-18763m22 | 42 | 4542 | 4543 |
| v4chr7-19301p126 | 42 | 4544 | 4545 |
| v4chr7-19898m9 | 42 | 4546 | 4547 |
| v4chr7-19989p31 | 42 | 4548 | 4549 |
| v4chr7-20119m35 | 42 | 4550 | 4551 |
| v4chr7-20129p4 | 42 | 4552 | 4553 |
| v4chr7-20184p30 | 42 | 4554 | 4555 |
| v4chr7-20372m10 | 42 | 4556 | 4557 |
| v4chr7-21169p2 | 42 | 4558 | 4559 |
| v4chr7-21255p7 | 42 | 4560 | 4561 |
| v4chr7-21306p37 | 42 | 4562 | 4563 |
| v4chr7-21580m9 | 42 | 4564 | 4565 |
| v4chr7-23200m20 | 42 | 4566 | 4567 |
| v4chr7-23223m11 | 42 | 4568 | 4569 |
| v4chr7-23429p5 | 42 | 4570 | 4571 |
| v4chr7-23530m22 | 42 | 4572 | 4573 |
| v4chr7-23604p13 | 42 | 4574 | 4575 |
| v4chr7-23619p18 | 42 | 4576 | 4577 |
| v4chr7-24121p22 | 42 | 4578 | 4579 |
| v4chr7-25156m15 | 42 | 4580 | 4581 |
| v4chr7-25165p25 | 42 | 4582 | 4583 |
| v4chr7-25302p11 | 42 | 4584 | 4585 |
| v4chr7-25369p27 | 42 | 4586 | 4587 |
| v4chr7-2719m29 | 42 | 4588 | 4589 |
| v4chr7-27430m24 | 42 | 4590 | 4591 |
| v4chr7-28352p22 | 42 | 4592 | 4593 |
| v4chr7-29772m26 | 42 | 4594 | 4595 |
| v4chr7-30284p14 | 42 | 4596 | 4597 |
| v4chr7-30555m3 | 42 | 4598 | 4599 |
| v4chr7-30883p11 | 42 | 4600 | 4601 |
| v4chr7-31201p15 | 42 | 4602 | 4603 |
| v4chr7-31252p29 | 42 | 4604 | 4605 |
| v4chr7-31302p41 | 42 | 4606 | 4607 |
| v4chr7-32054p14 | 42 | 4608 | 4609 |
| v4chr7-32431m10 | 42 | 4610 | 4611 |
| v4chr7-32564m3 | 42 | 4612 | 4613 |
| v4chr7-34674p27 | 42 | 4614 | 4615 |
| v4chr7-35046m19 | 42 | 4616 | 4617 |
| v4chr7-35124p9 | 42 | 4618 | 4619 |
| v4chr7-35178p34 | 42 | 4620 | 4621 |
| v4chr7-35261p5 | 42 | 4622 | 4623 |
| v4chr7-35281p13 | 42 | 4624 | 4625 |
| v4chr7-35332m11 | 42 | 4626 | 4627 |
| v4chr7-35342p9 | 42 | 4628 | 4629 |
| v4chr7-35544m12 | 42 | 4630 | 4631 |
| v4chr7-35861p13 | 42 | 4632 | 4633 |
| v4chr7-37199p165 | 42 | 4634 | 4635 |
| v4chr7-38264m86 | 42 | 4636 | 4637 |
| v4chr7-39308p29 | 42 | 4638 | 4639 |
| v4chr7-5064m17 | 42 | 4640 | 4641 |
| v4chr7-546p6 | 42 | 4642 | 4643 |
| v4chr7-5876p11 | 42 | 4644 | 4645 |
| v4chr7-6208p11 | 42 | 4646 | 4647 |
| v4chr7-7853p2 | 42 | 4648 | 4649 |
| v4chr7-7896p9 | 42 | 4650 | 4651 |
| v4chr7-7981m13 | 42 | 4652 | 4653 |
| v4chr7-8115m16 | 42 | 4654 | 4655 |
| v4chr7-8393m14 | 42 | 4656 | 4657 |
| v4chr7-8413p2 | 42 | 4658 | 4659 |
| v4chr7-8788p48 | 42 | 4660 | 4661 |
| v4chr7-9302m15 | 42 | 4662 | 4663 |
| v4chr7-9859p19 | 42 | 4664 | 4665 |
| v4chr7-9899m18 | 42 | 4666 | 4667 |
| v4chr7-9927m20 | 42 | 4668 | 4669 |
| v4chr7-9954m6 | 42 | 4670 | 4671 |
| v4s110-252m4 | 42 | 4672 | 4673 |
| v4s122-3p12 | 42 | 4674 | 4675 |
| v4s123-10m5 | 42 | 4676 | 4677 |
| v4s18-14m14 | 42 | 4678 | 4679 |
| v4s47-4m3 | 42 | 4680 | 4681 |
| v4s89-0p5 | 42 | 4682 | 4683 |
| v4chr1-28183m7 | 42 | 4684 | 4685 |
| v4chr1-38796p3 | 42 | 4686 | 4687 |
| v4chr1-47734p7 | 42 | 4688 | 4689 |
| v4chr2-30422m26 | 42 | 4690 | 4691 |
| v4chr2-38002m1 | 42 | 4692 | 4693 |
| v4chr2-41840m9 | 42 | 4694 | 4695 |
| v4chr2-47538p2 | 42 | 4696 | 4697 |
| v4chr2-54131p2 | 42 | 4698 | 4699 |
| v4chr2-55006p11 | 42 | 4700 | 4701 |
| v4chr2-57103m3 | 42 | 4702 | 4703 |
| v4chr2-6531m12 | 42 | 4704 | 4705 |
| v4chr2-67763m2 | 42 | 4706 | 4707 |
| v4chr3-14750p10 | 42 | 4708 | 4709 |
| v4chr3-15998p9 | 42 | 4710 | 4711 |
| v4chr3-21271p15 | 42 | 4712 | 4713 |
| v4chr3-21345m13 | 42 | 4714 | 4715 |
| v4chr3-25327m16 | 42 | 4716 | 4717 |
| v4chr3-35150m5 | 42 | 4718 | 4719 |
| v4chr3-36331m28 | 42 | 4720 | 4721 |
| v4chr3-45200p17 | 42 | 4722 | 4723 |
| v4chr3-6900m32 | 42 | 4724 | 4725 |
| v4chr3-8495p9 | 42 | 4726 | 4727 |
| v4chr4-16808m17 | 42 | 4728 | 4729 |
| v4chr4-25532m10 | 42 | 4730 | 4731 |
| v4chr4-25583p2 | 42 | 4732 | 4733 |
| v4chr4-28725p4 | 42 | 4734 | 4735 |
| v4chr4-3843m5 | 42 | 4736 | 4737 |
| v4chr4-45591p5 | 42 | 4738 | 4739 |
| v4chr4-49428p16 | 42 | 4740 | 4741 |
| v4chr5-17672m19 | 42 | 4742 | 4743 |
| v4chr5-20891m36 | 42 | 4744 | 4745 |
| v4chr6a-1658m7 | 42 | 4746 | 4747 |
| v4chr6a-16816m3 | 42 | 4748 | 4749 |
| v4chr6a-18888p5 | 42 | 4750 | 4751 |
| v4chr6a-21244p18 | 42 | 4752 | 4753 |
| v4chr6a-27786p3 | 42 | 4754 | 4755 |
| v4chr6a-33339p3 | 42 | 4756 | 4757 |
| v4chr6a-35341m3 | 42 | 4758 | 4759 |
| v4chr6a-35970m21 | 42 | 4760 | 4761 |
| v4chr6b-1650p3 | 42 | 4762 | 4763 |
| v4chr6b-9548p4 | 42 | 4764 | 4765 |
| v4chr7-2813m5 | 42 | 4766 | 4767 |
| v4s77-1p12 | 42 | 4768 | 4769 |
| v4chr4-49403p18 | 42 | 4770 | 4771 |
| v4chr4-49707p17 | 42 | 4772 | 4773 |
| v4chr1-35417m25 | 42 | 4774 | 4775 |
| v4chr1-39060m47 | 42 | 4776 | 4777 |
| v4chr2-71896m49 | 42 | 4778 | 4779 |
| v4chr3-36383m33 | 42 | 4780 | 4781 |
| v4chr3-39975m12 | 42 | 4782 | 4783 |
| v4chr4-49543m46 | 42 | 4784 | 4785 |
| v4chr5-4487m6 | 42 | 4786 | 4787 |
| v4chr6a-16517m23 | 42 | 4788 | 4789 |
| v4chr6a-5146m22 | 42 | 4790 | 4791 |
| v4chr6b-15533p58 | 42 | 4792 | 4793 |
| v4chr1-11270p4 | 42 | 4794 | 4795 |
| v4chr1-17287m10 | 42 | 4796 | 4797 |
| v4chr1-2075m3 | 42 | 4798 | 4799 |
| v4chr1-42846p13 | 42 | 4800 | 4801 |
| v4chr1-4705m12 | 42 | 4802 | 4803 |
| v4chr2-28619p23 | 42 | 4804 | 4805 |
| v4chr2-31374p19 | 42 | 4806 | 4807 |
| v4chr2-31714p14 | 42 | 4808 | 4809 |
| v4chr2-55320m10 | 42 | 4810 | 4811 |
| v4chr2-76305m4 | 42 | 4812 | 4813 |
| v4chr3-25487p16 | 42 | 4814 | 4815 |
| v4chr3-30790m26 | 42 | 4816 | 4817 |
| v4chr3-34456m5 | 42 | 4818 | 4819 |
| v4chr3-38704p16 | 42 | 4820 | 4821 |
| v4chr3-48588m17 | 42 | 4822 | 4823 |
| v4chr4-23468p27 | 42 | 4824 | 4825 |
| v4chr4-46266p13 | 42 | 4826 | 4827 |
| v4chr5-1818m12 | 42 | 4828 | 4829 |
| v4chr5-19468m5 | 42 | 4830 | 4831 |
| v4chr5-3741m4 | 42 | 4832 | 4833 |
| v4chr5-43547m5 | 42 | 4834 | 4835 |
| v4chr6a-31737m13 | 42 | 4836 | 4837 |
| v4chr5-2139m20 | 42 | 4838 | 4839 |
| v4chr6a-1436p16 | 42 | 4840 | 4841 |
| v4chr2-3052m26 | 42 | 4842 | 4843 |
| v4chr3-47862p24 | 42 | 4844 | 4845 |
| v4chr3-48039p19 | 42 | 4846 | 4847 |
| v4chr4-11467p23 | 42 | 4848 | 4849 |
| v4chr4-19669p20 | 42 | 4850 | 4851 |
| v4chr3-6089m22 | 42 | 4852 | 4853 |
| v4chr4-31072m24 | 42 | 4854 | 4855 |
| v4chr5-42621p14 | 42 | 4856 | 4857 |
| v4chr7-31425p11 | 42 | 4858 | 4859 |
| v4chr1-17237m20 | 42 | 4860 | 4861 |
| v4chr1-21037m8 | 42 | 4862 | 4863 |
| v4chr1-44782m31 | 42 | 4864 | 4865 |
| v4chr1-58432p20 | 42 | 4866 | 4867 |
| v4chr2-16357m11 | 42 | 4868 | 4869 |
| v4chr2-16754m10 | 42 | 4870 | 4871 |
| v4chr2-23009m14 | 42 | 4872 | 4873 |
| v4chr2-26516m19 | 42 | 4874 | 4875 |
| v4chr2-4114m47 | 42 | 4876 | 4877 |
| v4chr2-74738p9 | 42 | 4878 | 4879 |
| v4chr2-77174m11 | 42 | 4880 | 4881 |
| v4chr3-13141m27 | 42 | 4882 | 4883 |
| v4chr3-19594p8 | 42 | 4884 | 4885 |
| v4chr3-30413p3 | 42 | 4886 | 4887 |
| v4chr3-46575p8 | 42 | 4888 | 4889 |
| v4chr3-48620p23 | 42 | 4890 | 4891 |
| v4chr3-49007m21 | 42 | 4892 | 4893 |
| v4chr3-8010p14 | 42 | 4894 | 4895 |
| v4chr3-8628m44 | 42 | 4896 | 4897 |
| v4chr4-40403p18 | 42 | 4898 | 4899 |
| v4chr4-792p13 | 42 | 4900 | 4901 |
| v4chr5-21767p9 | 42 | 4902 | 4903 |
| v4chr5-22623m13 | 42 | 4904 | 4905 |
| v4chr5-22805p11 | 42 | 4906 | 4907 |
| v4chr5-22844m15 | 42 | 4908 | 4909 |
| v4chr5-26500p9 | 42 | 4910 | 4911 |
| v4chr5-29596m15 | 42 | 4912 | 4913 |
| v4chr5-41353m20 | 42 | 4914 | 4915 |
| v4chr6a-25767m5 | 42 | 4916 | 4917 |
| v4chr6b-2865m29 | 42 | 4918 | 4919 |
| v4chr6b-480p24 | 42 | 4920 | 4921 |
| v4chr7-18486m57 | 42 | 4922 | 4923 |
| v4chr7-27654m32 | 42 | 4924 | 4925 |
| v4chr7-36462p21 | 42 | 4926 | 4927 |
| v4chr7-40391m13 | 42 | 4928 | 4929 |
| v4chr7-4577m28 | 42 | 4930 | 4931 |
| v4chr1-15220p14 | 42 | 4932 | 4933 |
| v4chr1-20481m14 | 42 | 4934 | 4935 |
| v4chr1-39497m26 | 42 | 4936 | 4937 |
| v4chr1-41044p37 | 42 | 4938 | 4939 |
| v4chr1-46347m5 | 42 | 4940 | 4941 |
| v4chr1-52244p12 | 42 | 4942 | 4943 |
| v4chr1-6764p17 | 42 | 4944 | 4945 |
| v4chr1-9287p14 | 42 | 4946 | 4947 |
| v4chr2-16818p11 | 42 | 4948 | 4949 |
| v4chr2-20984p15 | 42 | 4950 | 4951 |
| v4chr2-40269p8 | 42 | 4952 | 4953 |
| v4chr2-43741p22 | 42 | 4954 | 4955 |
| v4chr2-49577p8 | 42 | 4956 | 4957 |
| v4chr2-8309p14 | 42 | 4958 | 4959 |
| v4chr3-27555p11 | 42 | 4960 | 4961 |
| v4chr3-30505p7 | 42 | 4962 | 4963 |
| v4chr3-42960m12 | 42 | 4964 | 4965 |
| v4chr3-6659m14 | 42 | 4966 | 4967 |
| v4chr3-6999p13 | 42 | 4968 | 4969 |
| v4chr3-7900m22 | 42 | 4970 | 4971 |
| v4chr4-19565m7 | 42 | 4972 | 4973 |
| v4chr4-3718m9 | 42 | 4974 | 4975 |
| v4chr4-37303p21 | 42 | 4976 | 4977 |
| v4chr5-18195m22 | 42 | 4978 | 4979 |
| v4chr5-19037p4 | 42 | 4980 | 4981 |
| v4chr6a-27300p34 | 42 | 4982 | 4983 |
| v4chr6a-4893m24 | 42 | 4984 | 4985 |
| v4chr6b-6711p11 | 42 | 4986 | 4987 |
| v4chr7-11753p10 | 42 | 4988 | 4989 |
| v4chr7-14666m29 | 42 | 4990 | 4991 |
| v4chr7-22362m39 | 42 | 4992 | 4993 |
| v4chr7-24379p47 | 42 | 4994 | 4995 |
| v4chr7-25210m10 | 42 | 4996 | 4997 |
| v4chr7-9827p9 | 42 | 4998 | 4999 |
| v4chr1-11812m30 | 42 | 5000 | 5001 |
| v4chr1-19706p9 | 42 | 5002 | 5003 |
| v4chr1-26694p4 | 42 | 5004 | 5005 |
| v4chr2-13564p23 | 42 | 5006 | 5007 |
| v4chr2-37835m8 | 42 | 5008 | 5009 |
| v4chr2-43530p23 | 42 | 5010 | 5011 |
| v4chr2-50773m19 | 42 | 5012 | 5013 |
| v4chr2-52124p20 | 42 | 5014 | 5015 |
| v4chr2-54153p27 | 42 | 5016 | 5017 |
| v4chr2-55438p9 | 42 | 5018 | 5019 |
| v4chr2-60405m27 | 42 | 5020 | 5021 |
| v4chr2-66176p10 | 42 | 5022 | 5023 |
| v4chr3-25027p19 | 42 | 5024 | 5025 |
| v4chr3-32644p33 | 42 | 5026 | 5027 |
| v4chr3-42134p28 | 42 | 5028 | 5029 |
| v4chr3-42284m2 | 42 | 5030 | 5031 |
| v4chr3-49534p24 | 42 | 5032 | 5033 |
| v4chr3-50473m36 | 42 | 5034 | 5035 |
| v4chr4-13002p8 | 42 | 5036 | 5037 |
| v4chr4-16904p29 | 42 | 5038 | 5039 |
| v4chr4-32229m3 | 42 | 5040 | 5041 |
| v4chr4-42838m36 | 42 | 5042 | 5043 |
| v4chr4-4637p24 | 42 | 5044 | 5045 |
| v4chr5-21713p25 | 42 | 5046 | 5047 |
| v4chr5-39442p7 | 42 | 5048 | 5049 |
| v4chr5-9311m27 | 42 | 5050 | 5051 |
| v4chr6a-27201p43 | 42 | 5052 | 5053 |
| v4chr6a-28811m32 | 42 | 5054 | 5055 |
| v4chr7-22838m24 | 42 | 5056 | 5057 |
| v4chr1-27885p17 | 42 | 5058 | 5059 |
| v4chr1-32547p5 | 42 | 5060 | 5061 |
| v4chr1-56795m24 | 42 | 5062 | 5063 |
| v4chr1-59435m20 | 42 | 5064 | 5065 |
| v4chr1-59666m19 | 42 | 5066 | 5067 |
| v4chr2-69434p8 | 42 | 5068 | 5069 |
| v4chr4-49391m18 | 42 | 5070 | 5071 |
| v4chr6a-16812m19 | 42 | 5072 | 5073 |
| v4chr6a-24556m32 | 42 | 5074 | 5075 |
| v4chr6a-33258m2 | 42 | 5076 | 5077 |
| v4chr6b-13964p20 | 42 | 5078 | 5079 |
| v4chr6b-529m20 | 42 | 5080 | 5081 |
| v4chr7-21520m40 | 42 | 5082 | 5083 |
| v4chr1-17162p13 | 42 | 5084 | 5085 |
| v4chr2-31596m13 | 42 | 5086 | 5087 |
| v4chr2-47013m6 | 42 | 5088 | 5089 |
| v4chr2-69495p14 | 42 | 5090 | 5091 |
| v4chr3-14486p16 | 42 | 5092 | 5093 |
| v4chr3-31750p9 | 42 | 5094 | 5095 |
| v4chr3-33624p117 | 42 | 5096 | 5097 |
| v4chr3-34183p12 | 42 | 5098 | 5099 |
| v4chr3-51222p8 | 42 | 5100 | 5101 |
| v4chr4-1825p8 | 42 | 5102 | 5103 |
| v4chr4-31019m18 | 42 | 5104 | 5105 |
| v4chr4-32437m20 | 42 | 5106 | 5107 |
| v4chr5-15332p25 | 42 | 5108 | 5109 |
| v4chr5-22660m9 | 42 | 5110 | 5111 |
| v4chr5-2485p20 | 42 | 5112 | 5113 |
| v4chr6a-15723m17 | 42 | 5114 | 5115 |
| v4chr1-2181p20 | 42 | 5116 | 5117 |
| v4chr1-38193m18 | 42 | 5118 | 5119 |
| v4chr6a-3996p11 | 42 | 5120 | 5121 |
| v4chr7-28844p31 | 42 | 5122 | 5123 |
| v4chr1-15715m8 | 42 | 5124 | 5125 |
| v4chr1-2502p3 | 42 | 5126 | 5127 |
| v4chr1-27550m2 | 42 | 5128 | 5129 |
| v4chr1-3994p3 | 42 | 5130 | 5131 |
| v4chr1-60195p3 | 42 | 5132 | 5133 |
| v4chr2-63086p3 | 42 | 5134 | 5135 |
| v4chr2-70323m3 | 42 | 5136 | 5137 |
| v4chr2-78398p2 | 42 | 5138 | 5139 |
| v4chr3-15343p3 | 42 | 5140 | 5141 |
| v4chr4-11909p3 | 42 | 5142 | 5143 |
| v4chr4-9158m3 | 42 | 5144 | 5145 |
| v4chr5-33493m2 | 42 | 5146 | 5147 |
| v4chr6a-13960p3 | 42 | 5148 | 5149 |
| v4chr6a-9805m2 | 42 | 5150 | 5151 |
| v4chr7-26755m2 | 42 | 5152 | 5153 |
| v4chr7-5397m2 | 42 | 5154 | 5155 |
| v4s43-15m3 | 42 | 5156 | 5157 |
| v4chr6a-2142p20 | 42 | 5158 | 5159 |
| v4chr1-51877m6 | 42 | 5160 | 5161 |
| v4chr7-18054p19 | 42 | 5162 | 5163 |
| v4chr4-49235m15 | 42 | 5164 | 5165 |
| v4chr7-9571p24 | 42 | 5166 | 5167 |
| v4chr1-54444m19 | 42 | 5168 | 5169 |
| v4chr7-11600m11 | 42 | 5170 | 5171 |
| v4chr3-35717m16 | 42 | 5172 | 5173 |
| v4chr2-28801m18 | 42 | 5174 | 5175 |
| v4chr2-32956p18 | 42 | 5176 | 5177 |
| v4chr6b-4957m19 | 42 | 5178 | 5179 |
| v4chr6a-8211p6 | 42 | 5180 | 5181 |
| v4chr6b-6644m57 | 42 | 5182 | 5183 |
| v4chr2-32429p32 | 42 | 5184 | 5185 |
| v4chr3-40652m25 | 42 | 5186 | 5187 |
| v4chr6a-10595m15 | 42 | 5188 | 5189 |
| v4chr7-23547p14 | 42 | 5190 | 5191 |
| v4chr3-46456p10 | 42 | 5192 | 5193 |
| v4chr4-33294p19 | 42 | 5194 | 5195 |
| v4chr6b-10102m17 | 42 | 5196 | 5197 |
| v4chr7-29402m28 | 42 | 5198 | 5199 |
| v4chr4-32556m5 | 42 | 5200 | 5201 |
| v4chr7-14887p22 | 42 | 5202 | 5203 |
| v4chr4-2008m11 | 42 | 5204 | 5205 |
| v4chr2-12876m10 | 42 | 5206 | 5207 |
| v4s108-9m9 | 42 | 5208 | 5209 |
| v4s14-26p3 | 42 | 5210 | 5211 |
| v4chr5-16080p14 | 10, 39 | 5212 | 5213 |
| v4chr5-16108m12 | 12, 13 | 5214 | 5215 |
| v4chr2-58871p23 | 17, 23 | 5216 | 5217 |
| v4chr2-22503p30 | 21, 32 | 5218 | 5219 |
| v4chr2-41064p24 | 22, 23 | 5220 | 5221 |
| v4chr5-11255p14 | 3, 4, 7, 9 | 5222 | 5223 |
| v4chr5-24655m32 | 3, 4, 7, 9, 16 | 5224 | 5225 |
| v4chr2-33243p31 | 3, 4, 7, 9, 16 | 5226 | 5227 |
| v4chr6a-17994p9 | 3, 4, 7, 9, 16 | 5228 | 5229 |
| v4chr3-26903m11 | 31, 42 | 5230 | 5231 |
| v4chr3-33149p6 | 36, 37 | 5232 | 5233 |
| v4chr5-40856p4 | 36, 37 | 5234 | 5235 |
| v4chr6a-2925p37 | 36, 37 | 5236 | 5237 |
| v4chr1-16374m30 | 36, 37 | 5238 | 5239 |
| v4chr6a-36211p9 | 36, 37 | 5240 | 5241 |
| v4chr3-4957m10 | 36, 37, 38 | 5242 | 5243 |
| v4chr3-53312m5 | 36, 38 | 5244 | 5245 |
| v4chr3-27903m14 | 36, 38 | 5246 | 5247 |
| v4chr6a-10511m22 | 36, 38 | 5248 | 5249 |
| v4chr7-14165m32 | 36, 38 | 5250 | 5251 |
| v4chr2-75516m28 | 36, 38 | 5252 | 5253 |
| v4chr2-5283m6 | 36, 38 | 5254 | 5255 |
| v4chr5-1700p3 | 36, 39 | 5256 | 5257 |
| v4chr6a-24726m18 | 36, 39 | 5258 | 5259 |
| v4chr6a-205353m8 | 36, 45 | 5260 | 5261 |
| v4chr6b-12873m15 | 38, 39 | 5262 | 5263 |
| v4chr6b-220p10 | 38, 39 | 5264 | 5265 |
| v4chr5-11449m18 | 38, 39 | 5266 | 5267 |
| v4chr3-17076p9 | 38, 39 | 5268 | 5269 |
| v4chr2-194p15 | 38, 39 | 5270 | 5271 |
| v4chr2-23365p16 | 38, 39 | 5272 | 5273 |
| v4chr3-18302p12 | 38, 39 | 5274 | 5275 |
| v4chr3-37265p15 | 38, 39 | 5276 | 5277 |
| v4chr3-5003m9 | 38, 39 | 5278 | 5279 |
| v4chr4-30622p8 | 38, 39 | 5280 | 5281 |
| v4chr5-2153p14 | 38, 39 | 5282 | 5283 |
| v4chr5-7322p10 | 38, 39 | 5284 | 5285 |
| v4chr6a-12589p10 | 38, 39 | 5286 | 5287 |
| v4chr5-132p12 | 38, 39 | 5288 | 5289 |
| v4chr6a-10987m11 | 38, 39 | 5290 | 5291 |
| v4chr6a-24358p14 | 38, 39 | 5292 | 5293 |
| v4chr6a-24383p15 | 38, 39 | 5294 | 5295 |
| v4chr5-20377p25 | 5, 12, 13, 17, 23, 29 | 5296 | 5297 |
| v4chr6b-13308m15 | 5, 17, 23 | 5298 | 5299 |
| v4chr3-13279m17 | 5, 17, 23, 31 | 5300 | 5301 |
| v4chr4-14213m6 | 5, 17, 23, 31 | 5302 | 5303 |
| v4chr2-335p26 | 8, 33 | 5304 | 5305 |

TABLE 5

| Activity # | Activity | GH or CE family |
|---|---|---|
| 3 | arabinofuranosidase | GH3, GH43, GH51, GH54, and GH62 |
| 4 | arabinofuranosidase from | GH3, GH43, GH51, GH54, and GH62 |
| | xylose | |
| 5 | xyloglucanase | GH5, GH12, GH16, GH44, and GH74 |
| 6 | α-glucuronidase | GH67 and GH115 |
| 7 | β-xylosidase | GH3, GH30, GH39, GH43, GH52, and GH54 |
| 8 | β-galactosidase | GH2 and GH42 |
| 9 | arabinofuranosidase/arabinase | GH3, GH43, GH51, GH54, GH62, and GH93 |
| 10 | chitin binding protein | |
| 11 | lichenan (β(1,3)-β(1,4)-linked | |
| | glucan) binding protein | |
| 12 | endo-xylanase | GH5, GH8, GH10, and GH11 |
| 13 | xylanase | GH5, GH8, GH10, and GH11 |
| 14 | xylan-binding protein | |
| 15 | polygalacturonase | GH28 |
| 16 | β-glucosidase | GH1, GH3, GH9, and GH30 |
| 17 | β-1,3-glucanase | GH5, GH12, GH16, GH17, GH55, GH64 and GH81 |
| 18 | α-1,6-Mannanase | GH38, GH76, and GH92 |
| 19 | Rhamnogalacturonyl hydrolase | GH28 and GH105 |
| 20 | α-Amylase | GH13 and GH57 |
| 21 | α-glucosidase | GH4, GH13, GH31, and GH63 |
| 22 | glucoamylase | GH15 |
| 23 | glucanase | GH5, GH6, GH7, GH8, GH9, GH12, GH13, GH14, |
| | | GH15, GH16, GH17, GH30, GH44, GH48, GH49, |
| | | GH51, GH55, GH57, GH64, GH71, GH74, GH81 |
| 24 | acetyl esterase | CEI, CE2, CE3, CE4, CE5, CE6, CE7, CE12, CE13 |
| | | and CEI6 |
| 25 | acetyl xylan esterases | CEI, CE2, CE3, CE4, CES, CE6, CE7, CEI2, and |
| | | CEI6 |
| 26 | ferulic acid esterase | |
| 27 | ferulic acid esterase | |
| 28 | glucuronyl esterase | possibly CE15 |
| 29 | endo-glucanase | GH5, GH6, CH7, GH8, GH9, GH12, GH44, GH45, |
| | | GH74 |
| 30 | α-glucanase | |
| 31 | β-glucanase | |
| 32 | α-galactosidase | |
| 33 | β-mannosidase | |
| 34 | rhamnogalacturonan acetyl | |
| | esterase | |
| 35 | protease | |
| 36 | oxidase | |
| 37 | peroxidase | |
| 38 | reductase | |
| 39 | dehydrogenase | |
| 40 | cutinase | |
| 41 | Pectin acetyl esterases or | |
| | Rhamnogalacturonan acetyl | |
| | esterase | |
| 42 | BCA assay & GOPOD assay | |
| 43 | Fucosidase | GH29 |
| 44 | Alpha-xylosidase | GH31 |
| 45 | laccase | |
| 46 | | |

1. A recombinant Myceliophthera thermophilus polypeptide comprising an amino acid sequence identified in any of Tables 1-4.

2. The recombinant polypeptide of claim 1, wherein the polypeptide is selected from the group consisting of a glycohydrolase, a carbohydrate esterase, an oxidase, an oxidoreductase a reductase and a dehydrogenase.

3. (canceled)

4. An isolated nucleic acid encoding a polypeptide of claim 1.

5. (canceled)

6. A recombinant vector comprising at least one nucleic acid of claim 4, wherein the nucleic acid is operably linked to a promoter.

7. (canceled)

8. A recombinant host cell comprising at least one recombinant vector of claim 6, operably linked to a heterologous promoter.

9. The recombinant host cell of claim 8, wherein the host cell is prokaryotic or eukaryotic.

10-14. (canceled)

15. A method of producing a polypeptide, the method comprising culturing a recombinant host cell of claim 8, under conditions in which the polypeptide is produced.

16-18. (canceled)

19. A method for degrading a cellulosic biomass, the method comprising contacting the cellulosic biomass with a composition comprising a recombinant biomass degradation polypeptide of claim 1.

20. The method of claim 19, wherein the composition is a cell culture medium into which the biomass degradation polypeptide has been secreted by cells expressing the polypeptide.

21-26. (canceled)

27. The method of claim 19, wherein the biomass degradation polypeptide is a glycohydrolase.

28. (canceled)

29. A composition comprising a cellulase and at least one recombinant biomass degradation polypeptide of claim 1.

30-32. (canceled)

33. A method of increasing production of active protein from a host cell, the method comprising modifying expression of a protein production polypeptide of any of Tables 1-4 in the host cell.

34-39. (canceled)
